(19) United States
(12) Patent Application Publication
(10) Pub. No.: US 2022/0017489 A1
(43) Pub. Date: Jan. 20, 2022

(54) SOLID DISPERSIONS FOR TREATMENT OF CANCER

(71) Applicant: Celgene Corporation, Summit, NJ (US)

(72) Inventors: Eric A. Fromme, Flemington, NJ (US); Kevin J. Klopfer, Flemington, NJ (US)

(21) Appl. No.: 17/290,224

(22) PCT Filed: Nov. 1, 2019
(86) PCT No.: PCT/US2019/059411

§ 371 Date: Apr. 30, 2021

Related U.S. Application Data
(60) Provisional application No. 62/755,165, filed on Nov. 2, 2018.

(51) Int. Cl. C07D 401/14; A61K 47/38; A61K 9/14

(52) U.S. Cl. CPC C07D 401/14; C07B 2200/13; A61K 9/14; A61K 47/38

(57) ABSTRACT

Provided herein are amorphous solid dispersions comprising Compound 1 and a polymer. Pharmaceutical compositions comprising the solid dispersions and methods for treating, preventing and managing cancer are also disclosed.

### RELATED APPLICATIONS

This application claims the benefit of U.S. Provisional Application No. 62/755,165, filed Nov. 2, 2018, the disclosure of which is incorporated herein by reference in its entirety.

### FIELD

Provided herein are solid dispersions comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a pharmaceutically acceptable salt, solvate, hydrate, stereoisomer, prodrug, or clathrate thereof, methods for preparing the solid dispersions and formulations comprising the same. In certain embodiments, the solid dispersions provided herein are used for treating a proliferative disease, such as cancer, characterized by the presence of a mutant allele of IDH2. In certain embodiments, the solid dispersions provided herein are amorphous.

### BACKGROUND ART

It has been reported that 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol is effective in treating proliferative diseases, including cancers. See U.S. Pat. Nos. 9,732,062; 9,738,625; 9,694,013; US Publication Nos. 2017/0157132; 2017/0246174; and International Publication Nos. WO 2015/017821; WO 2016/126798; and WO 2017/066599.

There is a need to develop formulations comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a pharmaceutically acceptable salt, solvate, hydrate, stereoisomer, prodrug, or clathrate thereof that have good manufacturability, dissolution, stability and bioavailability.

### SUMMARY

In certain embodiments, provided herein are solid dispersions comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a pharmaceutically acceptable salt, solvate, hydrate, stereoisomer, prodrug, or clathrate thereof (Compound 1) and a polymer. In certain embodiments, the solid dispersions are amorphous.

Also provided herein are methods of preparing the solid dispersions provided herein.

Also provided herein are pharmaceutical compositions comprising one or more solid dispersions provided herein.

Also provided herein are methods of treating and managing various diseases or disorders comprising administering to a patient a therapeutically effective amount of a solid dispersion provided herein.

In certain embodiments, provided herein are methods of treating hematological malignancies or solid tumors, each characterized by the presence of a mutant allele of IDH2 comprising administering an amorphous solid dispersion provided herein.

In one embodiment, the hematological malignancy is selected from acute myelogenous leukemia (AML), myelodysplastic syndrome (MDS), chronic myelomonocytic leukemia (CMML), myeloid sarcoma, multiple myeloma, lymphoma (e.g., T-cell lymphoma or B-cell lymphoma), angioimmunoblastic T-cell lymphoma (AITL), blastic plasmacytoid dendritic cell neoplasm and myeloproliferative neoplasm (MPN), each characterized by the presence of a mutant allele of IDH2.

In one embodiment, the solid tumor is selected from glioma, melanoma, chondrosarcoma, and cholangiocarcinoma, each characterized by the presence of a mutant allele of IDH2.

In certain embodiments, an amorphous solid dispersion provided herein is used for oral administration in patients for treating a proliferative disease, such as cancer, characterized by the presence of a mutant allele of IDH2.

In certain embodiments, an amorphous solid dispersion provided herein is used for oral administration in pediatric patients for treating a proliferative disease, such as cancer, characterized by the presence of a mutant allele of IDH2.

### BRIEF DESCRIPTION OF DRAWINGS

FIG. 1 provides an X-ray Powder Diffraction (“XRPD”) pattern of a mixture of solid Form A and Form 17 of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol (Compound 1A).

FIG. 2 provides a DSC spectrum of a mixture of solid Form A and Form 17 of Compound 1A.

FIG. 3 provides a TGMS spectrum of a mixture of solid Form A and Form 17 of Compound 1A.

FIG. 4 provides an HPLC profile of a mixture of solid Form A and Form 17 of Compound 1A.

FIG. 5 provides a mass spectrum of a mixture of solid Form A and Form 17 of Compound 1A.

FIG. 6 provides ¹H NMR spectrum of a mixture of solid Form A and Form 17 of Compound 1A.

FIG. 7 provides a graphical representation of the anti-precipitant screening of Compound 1A by the solvent shift method in simulated gastric fluid (SGF). The concentration of active pharmaceutical ingredient (API) in each SGF polymer solution is represented after 30 minutes, 1 hour, 2 hours, 4 hours and 24 hours. The dotted line represents the theoretical maximal API concentration (1.2 mg/mL).

FIG. 8 provides a graphical representation of the anti-precipitant screening of Compound 1A by the solvent shift method in simulated intestinal fluid (SIF).

FIG. 9 provides an overlay of HT-XRPD patterns of crystalline Compound 1A and solid dispersions prepared with Carbopol® 980 NF.

FIG. 10 provides a DSC spectrum of the solid dispersion for Compound 1A prepared with Carbopol® 980 NF and 10% drug load in dichloromethane (DCM).

FIG. 11 provides a DSC spectrum of the solid dispersion for Compound 1A prepared with Carbopol® 980 NF, 10% drug load in THF/water (50/50).

FIG. 12 provides a DSC spectrum analysis of the solid dispersion for Compound 1A prepared with Carbopol® 980 NF, 25% drug load in DCM.

FIG. 13 provides a DSC spectrum of the solid dispersion for Compound 1A prepared with Carbopol® 980 NF, 25% drug load in THF/water (50/50).

FIG. 14 provides a DSC spectrum of the solid dispersion for Compound 1A prepared with Carbopol® 980 NF, 50% drug load in methanol/water (90/10).

FIG. 15 provides a DSC spectrum of the solid dispersion for Compound 1A prepared with Carbopol® 980 NF, 50% drug load in THF/water (50/50).

FIG. 16 provides a TGMS spectrum of the solid dispersion for Compound 1A prepared with Carbopol® 980 NF, 10% drug load in DCM.

FIG. 17 provides a TGMS spectrum of the solid dispersion for Compound 1A prepared with Carbopol® 980 NF, 10% drug load in THF/water (50/50).

FIG. 18 provides a TGMS spectrum of the solid dispersion for Compound 1A prepared with Carbopol® 980 NF, 25% drug load in DCM.

FIG. 19 provides a TGMS spectrum of the solid dispersion for Compound 1A prepared with Carbopol® 980 NF, 25% drug load in THF/water (50/50).

FIG. 20 provides a TGMS spectrum of the solid dispersion for Compound 1A prepared with Carbopol® 980 NF, 50% drug load in methanol/water (90/10).

FIG. 21 provides a TGMS spectrum of the solid dispersion for Compound 1A prepared with Carbopol® 980 NF, 50% drug load in THF/water (50/50).

FIG. 22 provides an overlay of HT-XRPD patterns Compound 1A and solid dispersions for Compound 1A prepared with HPMC, 10% drug load in THF/water (50/50), 25% drug load in THF/water (50/50) and 50% drug load in acetone/methanol (80/20).

FIG. 23 provides a DSC spectrum of the solid dispersions for Compound 1A prepared with HPMC, 10% drug load in THF/water (50/50).

FIG. 24 provides a DSC spectrum of the solid dispersions for Compound 1A prepared with HPMC, 25% drug load in THF/water (50/50).

FIG. 25 provides a DSC spectrum of the solid dispersions for Compound 1A prepared with HPMC, 50% drug load in acetone/methanol (80/20).

FIG. 26 provides a TGMS spectrum of the solid dispersions for Compound 1A prepared with HPMC, 10% drug load in THF/water (50/50).

FIG. 27 provides a TGMS spectrum of the solid dispersions for Compound 1A prepared with HPMC, 25% drug load in THF/water (50/50).

FIG. 28 provides a TGMS spectrum of the solid dispersions for Compound 1A prepared with HPMC, 50% drug load in acetone/methanol (80/20).

FIG. 29 provides an overlay of HT-XRPD patterns (from bottom to top): crystalline Compound 1A and solid dispersions prepared with methyl cellulose, 10% drug load in THF/water (50/50), 25% drug load in chloroform/ethanol (50/50) (Exp. ID ASD41) and 50% drug load in THF/water (50/50) (Exp. ID ASD45).

FIG. 30 provides a DSC spectrum of the solid dispersion for Compound 1A prepared with methyl cellulose, 10% drug load in THF/water (50/50).

FIG. 31 provides a DSC spectrum of the solid dispersion for Compound 1A prepared with methyl cellulose, 25% drug load in chloroform/ethanol (50/50).

FIG. 32 provides a DSC spectrum of the solid dispersion for Compound 1A prepared with methyl cellulose, 50% drug load in THF/water (50/50).

FIG. 33 provides a TGMS spectrum of the solid dispersion for Compound 1A prepared with methyl cellulose, 10% drug load in THF/water (50/50).

FIG. 34 provides a TGMS spectrum of the solid dispersion for Compound 1A prepared with methyl cellulose, 25% drug load in chloroform/ethanol (50/50).

FIG. 35 provides a TGMS spectrum of the solid dispersion for Compound 1A prepared with methyl cellulose, 50% drug load in THF/water (50/50).

FIG. 36 provides a graphical representation of the solid dispersion for Compound 1A prepared with Carbopol® 980 NF in three solvents systems before and after stability for 4 weeks. The loss on drying determined by TGMS analysis on the solid dispersions is presented above each bar.

FIG. 37 provides a graphical representation of the solid dispersion for Compound 1A prepared with HPMC in three solvents systems before and after stability for 4 weeks. The loss on drying determined by TGMS analysis on the solid dispersions is presented above each bar.

FIG. 38 provides a graphical representation of the solid dispersion for Compound 1A prepared with methyl cellulose in three solvents systems before and after stability for 4 weeks. The loss on drying determined by TGMS analysis is presented above each bar.

FIG. 39 provides a graphical representation of the solubility results of solid dispersions for Compound 1A in water at 37° C.

FIG. 40 provides a graphical representation of the solubility results of solid dispersions for Compound 1A in SIF at 37° C.

FIG. 41 provides a graphical representation of the solubility results of solid dispersions for Compound 1A in SGF at 37° C.

FIG. 42 provides a graphical representation the relative solubility of Compound 1A in SGF (37° C.) expressed as percentage of the maximal achievable concentration when all of Compound 1A from the dispersion had gone into solution.

FIG. 43 provides X-ray Powder Diffraction pattern of Form 3 of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate (Compound 1B).

FIG. 44 provides a graphical representation of the Whole Powder Pattern Decomposition (Pawley) analysis Compound 1B powder pattern.

FIG. 45 provides a DSC spectrum of Form 3 of Compound 1B.

FIG. 46 provides a TGMS spectrum of Form 3 of Compound 1B.

FIG. 47 provides an HPLC profile of Form 3 of Compound 1B.

FIG. 48 provides a mass spectrum of Form 3 of Compound 1B.

FIG. 49 provides 1H NMR spectrum of Form 3 of Compound 1B.

FIG. 50 provides a graphical representation of the anti-precipitant screening of Compound 1B by the solvent shift method in SGF.

FIG. 51 provides a graphical representation of the anti-precipitant screening of Compound 1B by the solvent shift method in SIF.

FIG. 52 provides an overlay of HT-XRPD patterns (from bottom to top): crystalline Compound 1B, amorphous solid dispersion prepared with Carbopol® 980 NF and 10% drug load in THF/water (50/50) before and after exposure to accelerated aging conditions for 4 weeks.

FIG. 53 provides a DSC spectrum of the amorphous solid dispersion for Compound 1B prepared with Carbopol® 980 NF and 10% drug load in THF/water (50/50).

FIG. 54 provides a TGMS spectrum of amorphous solid dispersion for Compound 1B prepared with Carbopol® 980 NF and 10% drug load in THF/water (50/50).

FIG. 55 provides an overlay of HTXRPD patterns (from bottom to top): crystalline Compound 1B, amorphous solid dispersion prepared for Compound 1B with HPMC and 25% drug load in TFE/water (80/20) before and after exposure to accelerated aging conditions (40° C./70% RH) for 4 weeks.

FIG. 56 provides a DSC spectrum of the amorphous solid dispersion prepared for Compound 1B with HPMC and 25% drug load in TFE/water (80/20).

FIG. 57 provides a TGMS spectrum of the amorphous solid dispersion for Compound 1B prepared with HPMC and 25% drug load in TFE/water (80/20).

FIG. 58 provides an overlay of HT-XRPD patterns (from bottom to top): crystalline Compound 1B, amorphous solid dispersion prepared for Compound 1B with methyl cellulose and 10% drug load in chloroform/ethanol (50/50) before and after exposure to accelerated aging conditions (40° C./70% RH) for 4 weeks.

FIG. 59 provides a DSC spectrum of the amorphous solid dispersion for Compound 1B obtained with 10% methyl cellulose in chloroform/ethanol (50/50).

FIG. 60 provides a TGMS spectrum of the amorphous solid dispersion for Compound 1B obtained with 10% methyl cellulose in chloroform/ethanol (50/50).

FIG. 61 provides a graphical representation of the solid dispersions for Compound 1B prepared with Carbopol® 980 NF in three solvents systems before and after stability for 4 weeks.

FIG. 62 provides a graphical representation of the solid dispersions for Compound 1B prepared with HPMC in three solvents systems before and after stability for 4 weeks. The loss on drying determined by TGMS analysis on the amorphous solid dispersions is presented above each bar.

FIG. 63 provides a graphical representation of the solid dispersion for Compound 1B prepared with methyl cellulose in three solvents systems before and after stability for 4 weeks. The loss on drying determined by TGMS analysis is presented above each bar.

FIG. 64 provides a graphical representation of the solubility results of Compound 1B solid dispersions in water at 37° C.

FIG. 65 provides a graphical representation of the solubility results of Compound 1B solid dispersions in SIF at 37° C.

FIG. 66 provides a graphical representation of the solubility results of Compound 1B solid dispersions in SGF at 37° C.

FIG. 67 provides a graphical representation the relative solubility of Compound 1B in SGF (37° C.) expressed as percentage of the maximal achievable concentration when all API from the dispersion had gone into solution.

FIG. 68 provides an XRPD pattern for solid Form A of Compound 1A.

FIG. 69 provides TGA/DSC spectra for solid Form A of Compound 1A.

FIG. 70 provides ¹H NMR spectrum for solid Form A of Compound 1A.

FIG. 71 provides an XRPD pattern for solid Form G of Compound 1A.

FIG. 72 provides TGA/DSC spectra for solid Form G of Compound 1A.

FIG. 73 provides ¹H NMR spectrum for solid Form G of Compound 1A.

FIG. 74 provides an XRPD pattern for solid Form M5 of Compound 1B.

FIG. 75 provides an XRPD pattern for solid Form M8 of Compound 1B.

### DETAILED DESCRIPTION

The details of construction and the arrangement of components set forth in the following description or illustrated in the drawings are intended to describe non-limiting embodiments. Other embodiments and different ways to practice the invention are expressly included. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Definitions

As used above, and throughout the description of the invention, the following terms, unless otherwise indicated, shall be understood to have the following meanings.

As used in this application, the singular form “a”, “an” and “the” include plural references unless the context clearly dictates otherwise. For example, the term “an intragranular excipient” includes one or more intragranular excipients.

“Compound 1” is meant to describe 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or solvates, hydrates, stereoisomers, prodrugs, or clathrates thereof 2-Methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate (or mesylate) is also known as enasidenib.

The terms “AG 221” or “AG221” refer to 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol, including solid forms thereof, or 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate, including solid forms thereof.

2-Methyl-1-[(4-[6-(trifluoromethyl)pyridin-2-yl]-6-{[2-(trifluoromethyl)pyridin-4-yl]amino}-1,3,5-triazin-2-yl)amino]propan-2-ol is currently marketed in the U.S. by Celgene Corporation, as once-daily oral tablets for the treatment of adult patients with relapsed or refractory acute myeloid leukemia (AML) who have an IDH2 mutation, under the trade name IDHIFA®.

“Compound 1A” as used herein refers to 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol, including solid forms thereof.

“Compound 1B” as used herein refers to 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate, including solid forms thereof.

The term “solid form” refers a crystal form or an amorphous form or a mixture thereof of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a methanesulfonate salt thereof. Certain solid forms of 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol and its methanesulfonate salt are described in U.S. Pat. No. 9,738,625 and International Publication No. WO 2016/126798, each of which is incorporated by reference in its entirety.

As used herein, and unless otherwise specified, a crystalline or amorphous form that is “pure,” i.e., substantially free of other crystalline or amorphous forms, contains less than about 10% by weight of one or more other crystalline or amorphous forms, less than about 5% by weight of one or more other crystalline or amorphous forms, less than about 3% by weight of one or more other crystalline or amorphous forms, or less than about 1% by weight of one or more other crystalline or amorphous forms.

As used herein, and unless otherwise specified, a solid form that is “substantially physically pure” is substantially free from other solid forms. In certain embodiments, a crystal form that is substantially physically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In certain embodiments, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

As used herein, and unless otherwise indicated, a chemical compound, solid form, or composition that is “substantially free” of another chemical compound, solid form, or composition means that the compound, solid form, or composition contains, in certain embodiments, less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

As used herein, and unless otherwise specified, the term “pharmaceutically acceptable salts” refers to salts prepared from pharmaceutically acceptable, relatively non-toxic acids, including inorganic acids and organic acids. In some embodiments, suitable acids include, but are not limited to, acetic, benzenesulfonic, benzoic, camphorsulfonic, carbonic, citric, dihydrogenphosphoric, ethenesulfonic, fumaric, galactunoric, gluconic, glucuronic, glutamic, hydrobromic, hydrochloric, hydriodic, isobutyric, isethionic, lactic, maleic, malic, malonic, mandelic, methanesulfonic, monohydrogencarbonic, monohydrogen-phosphoric, monohydrogensulfuric, mucic, nitric, pamoic, pantothenic, phosphoric, phthalic, propionic, suberic, succinic, sulfuric, tartaric, toluenesulfonic acid (including p-toluenesulfonic, m-toluenesulfonic, and o-toluenesulfonic acids), and the like (see, e.g., S. M. Berge et al., J. Pharm. Sci., 66:1-19 (1977); and Handbook of Pharmaceutical Salts: Properties, Selection and Use, P. H. Stahl and C. G. Wermuth, Eds., (2002), Wiley, Weinheim). In some embodiments, suitable acids are strong acids (e.g., with pKa less than about 1), including, but not limited to, hydrochloric, hydrobromic, sulfuric, nitric, methanesulfonic, benzene sulfonic, toluene sulfonic, naphthalene sulfonic, naphthalene disulfonic, pyridine-sulfonic, or other substituted sulfonic acids. Also included are salts of other relatively non-toxic compounds that possess acidic character, including amino acids, such as aspartic acid and the like, and other compounds, such as aspirin, ibuprofen, saccharin, and the like. Acid addition salts can be obtained by contacting the neutral form of a compound with a sufficient amount of the desired acid, either neat or in a suitable solvent. As solids, salts can exist in crystalline or amorphous forms, or mixtures thereof. Salts can also exist in polymorphic forms.

Unless otherwise specified, the terms “solvate” and “solvated,” as used herein, refer to a solid form of a substance which contains solvent. The terms “hydrate” and “hydrated” refer to a solvate wherein the solvent is water. “Polymorphs of solvates” refer to the existence of more than one solid form for a particular solvate composition. Similarly, “polymorphs of hydrates” refer to the existence of more than one solid form for a particular hydrate composition. The term “desolvated solvate,” as used herein, refers to a solid form of a substance which can be made by removing the solvent from a solvate. The terms “solvate” and “solvated,” as used herein, can also refer to a solvate of a salt, co-crystal, or molecular complex. The terms “hydrate” and “hydrated,” as used herein, can also refer to a hydrate of a salt, co-crystal, or molecular complex.

Unless otherwise specified, the term “solid dispersion”, as used herein, refers to a solid state which comprises at least two constituents, wherein one constituent is homogenously dispersed significantly evenly throughout the other constituent or constituents. It includes solid or glassy solutions, i.e., the dispersion of the constituents is in such a way that the composition is chemically and physically homogenous in nature. In one embodiment, the first constituent is an active pharmaceutical ingredient (API), and the second constituent is a matrix that comprises a polymer, wherein the API is dispersed significantly uniformly within the matrix (the polymer). In one embodiment, the API is Compound 1. In one embodiment, the API is Compound 1A. In one embodiment, the API is Compound 1B. The API may be present in an amorphous state or in fine crystalline dispersed form. Also, the API may be available as a mixture of amorphous and crystalline forms. A solid dispersion can comprise more than two constituents. For example, two or more API can be dispersed into the matrix, and the matrix can comprise two or more polymers. Without limitation, solid dispersions may be physically classified as a eutectic mixture, a solid solution, a glass solution or suspension, an amorphous precipitate in a glassy or crystalline carrier, a complex, a complexed formation or a combination of the different systems. In addition, solid dispersions may be prepared using various techniques known to those skilled in the art, such as by co-dissolving the API and polymer in a solvent then spray-drying, spray-congealing, evaporation, freeze-evaporation, curing or microwaving, blending and direct compression, mechanical admixture at an elevated but non-melting temperature, wet granulation, extrusion-spheronization, melt fusion, hot melt extrusion and the like. A “solid matrix” refers to a matrix that is solid.

Unless otherwise specified, the term “polymer”, as used herein, refers to a compound comprising repeating structural units (monomers) connected by covalent chemical bonds. Polymers may be further derivatized, crosslinked, grafted or end-capped. Non-limiting examples of polymers include copolymers, terpolymers, quaternary polymers, and homologues. The term “copolymer” refers to a polymer consisting essentially of two or more different types of repeating structural units (monomers).

As used herein, the terms “inhibit” or “prevent” include both complete and partial inhibition and prevention. An inhibitor may completely or partially inhibit the intended target.

The term “treat” means decrease, suppress, attenuate, diminish, arrest, or stabilize the development or progression of a disease/disorder (i.e., a disease such as AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, lessen the severity of the disease/disorder (i.e., a disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma), each characterized by the presence of a mutant allele of IDH2, or improve the symptoms associated with the disease/disorder (i.e., a disease such as AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, or cholangiocarcinoma), each characterized by the presence of a mutant allele of IDH2.

As used herein, and unless otherwise specified, the terms “prevent,” “preventing” and “prevention” refer to the prevention of the onset, recurrence or spread of a disease or disorder, or of one or more symptoms thereof. The terms “prevent,” “preventing” and “prevention” contemplate an action that occurs before a patient begins to suffer from the specified disease or disorder or symptoms thereof, which inhibits or reduces the severity of the disease or disorder.

As used herein, and unless otherwise indicated, the terms “manage,” “managing” and “management” encompass preventing the recurrence of the specified disease or disorder in a patient who has already suffered from the disease or disorder, or lengthening the time that a patient who has suffered from the disease or disorder remains in remission. The terms encompass modulating the threshold, development or duration of the disease or disorder, or changing the way that a patient responds to the disease or disorder.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, that provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or one or more symptoms thereof, or prevent the recurrence of the disease or disorder, or one or more symptoms thereof. A prophylactically effective amount of a compound means an amount of therapeutic agent, alone or in combination with other agents, that provides a prophylactic benefit in the prevention of the disease or disorder. The term “prophylactically effective amount” can encompass an amount that improves overall prophylaxis or enhances the prophylactic efficacy of another prophylactic agent.

Unless otherwise specified, the term “composition” as used herein is intended to encompass a product comprising the specified ingredient(s) (and in the specified amount(s), if indicated), as well as any product which results, directly or indirectly, from combination of the specified ingredient(s) in the specified amount(s).

A “pharmaceutically acceptable excipient, diluent or carrier,” refers to a substance that aids the administration of an active agent to a subject by for example by modifying the stability of an active agent or modifying the absorption by a subject upon administration. A pharmaceutically acceptable excipient typically has no significant adverse toxicological effect on the patient. Examples of pharmaceutically acceptable excipients include, for example bulking agents, buffers, binders, fillers, disintegrants, lubricants, coatings, sweeteners, flavors, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6^{th} Ed., The Pharmaceutical Press, RPS Publishing (2009). The terms “bulking agent”, and “buffer” are used in accordance with the plain and ordinary meaning within the art.

As used herein, “administer” or “administration” refers to the act of physically delivering a substance as it exists outside the body into a subject. Administration includes all forms known in the art for delivering therapeutic agents, including but not limited to oral, topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

The term “co-administer” as used herein with respect to an additional cancer therapeutic agents means that the additional cancer therapeutic agent may be administered prior to, consecutively with, or following the administration of a composition provided herein. In such combination therapy treatment, the second therapeutic agent(s) is administered by conventional methods.

The terms “subject,” “patient,” “subject in need thereof,” and “patient in need thereof” are herein used interchangeably and refer to a living organism suffering from one or more of the diseases described herein (e.g., AML) that can be treated by administration of a composition described herein. Non-limiting examples of organisms include humans, other mammals, bovines, rats, mice, dogs, monkeys, goat, sheep, cows, deer, and other non-mammalian animals. In embodiments, a subject is human. A human subject can be between the ages of about 1 year old to about 100 years old. In embodiments, subjects herein can be characterized by the disease being treated (e.g., a “AML subject”, a “cancer subject”, or a “leukemia subject”).

As used herein, the term “pediatric patient” refers to a patient 21 years or younger, in certain embodiments, a patient 18 years or younger, in certain embodiments, a patient 16 years or younger, in certain embodiments, a patient 14 years or younger, in certain embodiments, a patient 12 years or younger, in certain embodiments, a patient 10 years or younger, or in certain embodiments, a patient 8 years or younger.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 20 while still describing the particular XRPD peak.

Unless otherwise specified, to the extent that there is a discrepancy between a depicted chemical structure of a compound provided herein and a chemical name of a compound provided herein, the chemical structure shall control.

Compound

In certain embodiments, provided herein is a solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol having the following formula:

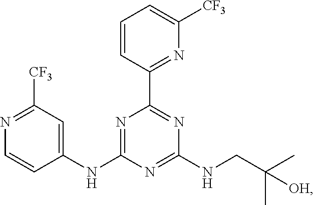 [SMILES: CC(C)(O)CNc1nc(Nc2ccnc(C(F)(F)F)c2)nc(-c2cccc(C(F)(F)F)n2)n1]

or a pharmaceutically acceptable salt, solvate, hydrate, stereoisomer, prodrug, or clathrate thereof (collectively referred to as Compound 1), and a polymer.

In certain embodiments, provided herein is a solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol (Compound 1A).

In certain embodiments, provided herein is a solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate (Compound 1B).

In one embodiment, Compound 1A used in the solid dispersions provided herein is a crystalline solid. In one embodiment, the solid dispersions provided herein comprise a mixture of polymorph forms of Compound 1A. In one embodiment, the solid dispersions provided herein comprise a mixture of two polymorph forms of Compound 1A. In one embodiment, the solid dispersions provided herein comprise a mixture of polymorph Form 17 and polymorph Form A of Compound 1A.

In one embodiment, Compound 1B used in the solid dispersions provided herein is a crystalline solid. In one embodiment, the solid dispersions provided herein comprise Form 3 of Compound 1B.

In one embodiment, Compound 1A and Compound 1B can be synthesized using methods described in U.S. Pat. Nos. 9,512,107; 9,656,999; 9,732,062; 9,738,625; 9,751,863 and U.S. Publication No. 2017/0305885 A1, and PCT Publication No. WO 2016/126798, all of which are incorporated herein in their entireties.

The polymorphic forms of Compound 1A and Compound 1B, including Form 17 of Compound 1A and Form 3 of Compound 1B, are described in U.S. Pat. No. 9,738,625 and WO 2016/126798, the entirety of which is incorporated herein by reference. Polymorph Form A of Compound 1A is described elsewhere herein.

### Form A of Compound 1A

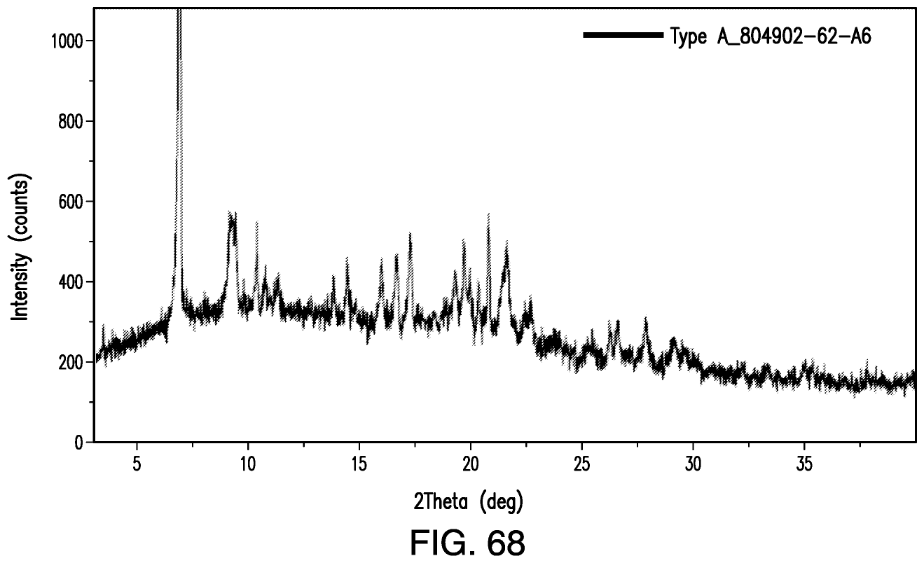

In one embodiment, a single crystalline form, Form A, of Compound 1A is characterized by the X-ray powder diffraction (XRPD) pattern shown in FIG. 68 obtained using CuKa radiation.

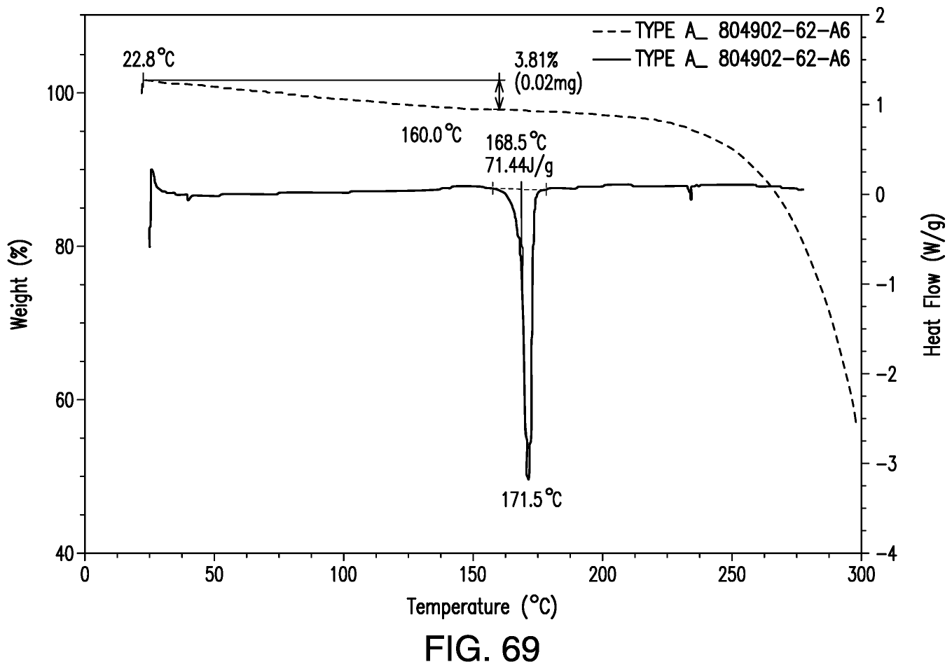

In another embodiment, Form A is characterized by the differential scanning calorimetry profile (DSC) shown in FIG. 69. The DSC graph plots the heat flow as a function of temperature from a sample, the temperature rate change being about 10° C./min. The profile is characterized by one endotherm at 168.5° C. (onset temperature).

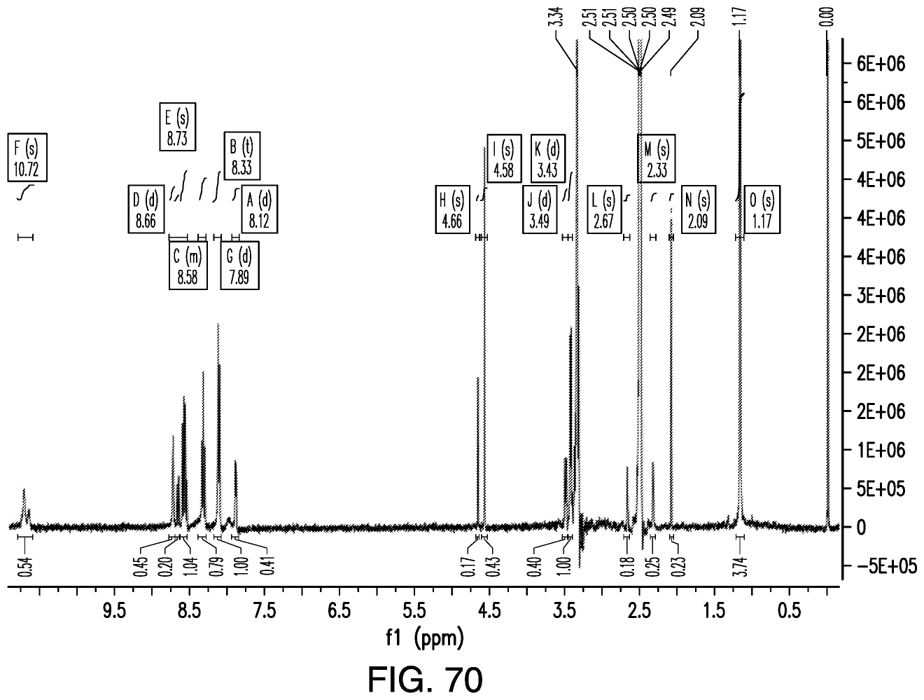

In another embodiment, Form A is characterized by thermal gravimetric analysis (TGA) shown in FIG. 69. The TGA profile graphs the percent loss of weight of the sample as a function of temperature, the temperature rate change being about 10° C./min. The weight loss represents a loss of about 3.8% of the weight of the sample as the temperature is increased to about 160.0° C. FIG. 70 provides the ¹H NMR spectrum, which indicates that the molar ratio of acetone to Form A is 0.06 due to the existence of residual solvent.

### Form G of Compound 1A

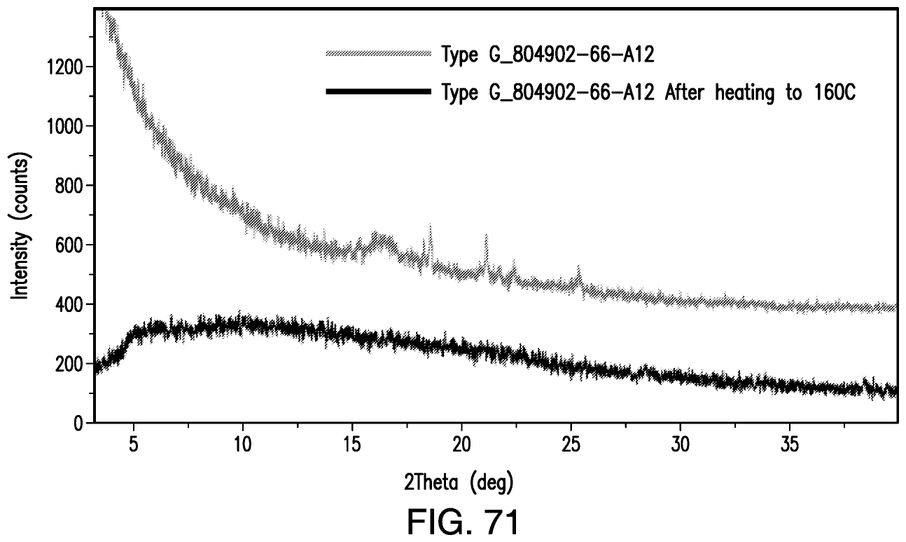

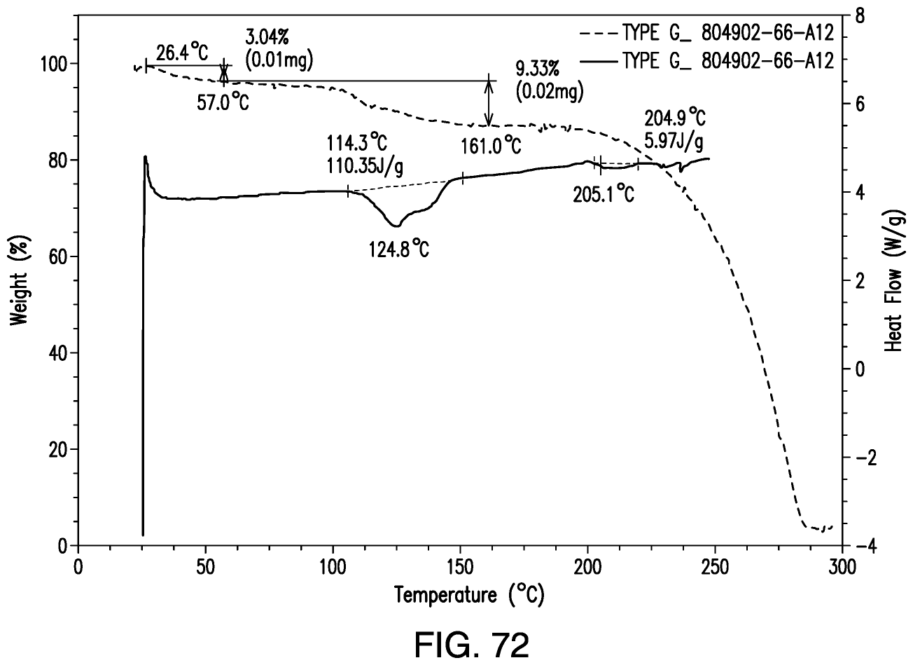

In one embodiment, a single crystalline form, Form G, of Compound 1A is characterized by the XRPD pattern shown in FIG. 71 obtained using CuKa radiation. In another embodiment, Form G is characterized by the DSC shown in FIG. 72. The DSC graph plots the heat flow as a function of temperature from a sample, the temperature rate change being about 10° C./min. The profile is characterized by two endotherms at 114.3° C. and 204.9° C. (onset temperature).

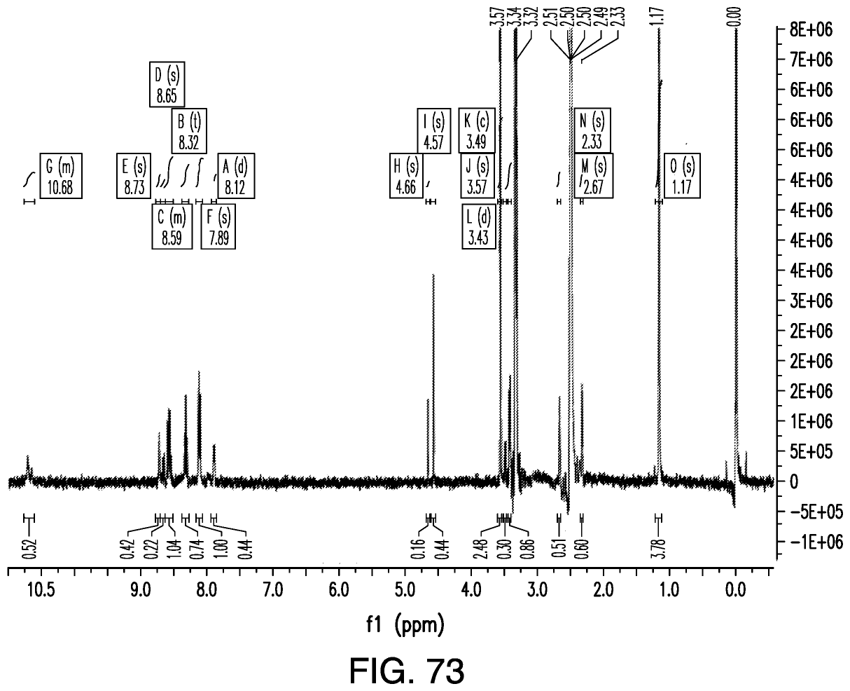

In another embodiment, Form A is characterized by TGA shown in FIG. 72. The TGA profile graphs the percent loss of weight of the sample as a function of temperature, the temperature rate change being about 10° C./min. The weight loss represents a loss of about 12.4% of the weight of the sample as the temperature is increased to about 160.0° C. When heated to 160° C., Type G converted to amorphous, as shown in FIG. 71. FIG. 73 provides the ¹H NMR spectrum, which indicates that the molar ratio of dioxane to Form A is 0.5.

### Form M5 of Compound 1B

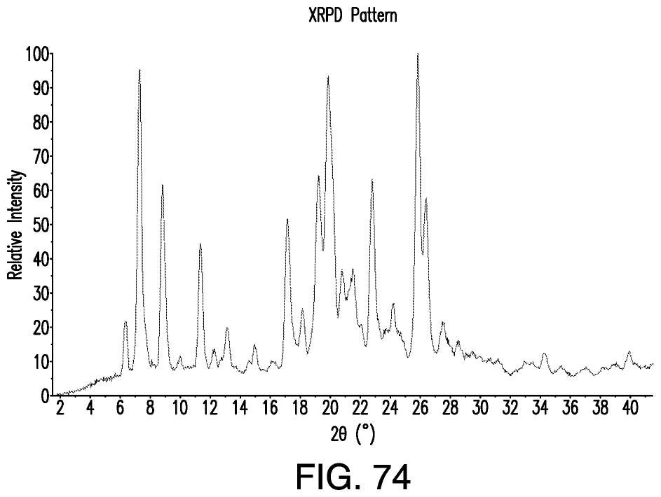

Form M5 of Compound 1B is a mixture of Form 14, Form 15 and Form 3 of Compound 1B. Forms 14, 15 and 3 of Compound 1B are described in U.S. Pat. No. 9,738,625. In one embodiment, Form M5, of Compound 1B is characterized by the XRPD pattern shown in FIG. 74 obtained using CuKa radiation.

### Form M8 of Compound 1B

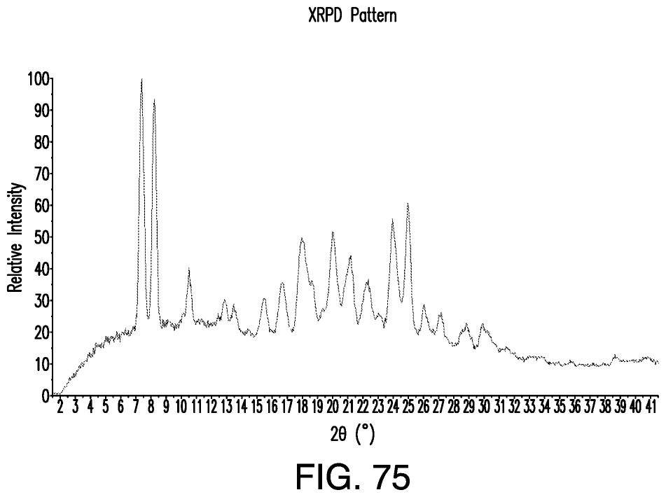

Form M8 of Compound 1B comprises mainly Form 12 of Compound 1B. Form 12 of Compound 1B is described in U.S. Pat. No. 9,738,625. In one embodiment, Form M8, of Compound 1B is characterized by the XRPD pattern shown in FIG. 75 obtained using CuKa radiation.

Polymers

In one embodiment, the polymer is selected from cellulose esters and cellulose ethers, polyalkylene oxides, polyacrylates and polymethacrylates, homopolymers and copolymers of N-vinyl lactams, polyacrylamides, vinyl acetate polymers, graft copolymers of polyethylene glycol, polyvinyl caprolactam and polyvinyl acetate, polyvinyl acetate phthalate, oligo- and polysaccharides, and mixtures of two or more thereof.

In one embodiment, the polymer is selected from hydroxyethyl cellulose, hydroxypropyl methyl cellulose, hydroxypropyl cellulose, Eudragit E100 (methacrylate copolymer), polyethylene glycol 6000, polyvinylpyrrolidone K30, poly(1-vinylpyrrolidone-co-vinyl acetate, Pluronic F-68 (polyoxyethylene-polyoxypropylene copolymer), D-α-tocopherol polyethylene glycol 1000 succinate, acrylic polymer (Carbopol® 980 NF Polymer), polyvinyl caprolactam-polyvinyl acetate-polyethylene glycol graft copolymer (Soluplus®), polyvinyl alcohol-acrylic acid-methyl methacrylate copolymer (Povacoat Type MP), polyvinyl alcohol/polyethylene glycol graft copolymer (Kollicoat IR), polyvinyl acetate phthalate (Sureteric®) and methyl cellulose

In one embodiment, the polymer is selected from Hydroxyethyl cellulose (HEC), Hydroxypropyl methyl cellulose (HPMC), Hydroxypropyl cellulose (HPC), Eudragit E100, Polyethylene Glycol 6000 (PEG 6000), Polyvinylpyrrolidone K30 (PVP K30), Poly(1-vinylpyrrolidone-co-vinyl acetate) (PVP-VA), Pluronic F-68, D-α-Tocopherol polyethylene glycol 1000 succinate (TPGS), Carbopol® 980 NF Polymer, Soluplus®, Povacoat Type MP, Kollicoat IR, Sureteric® and Methyl cellulose.

In one embodiment, the polymer is Soluplus®, Carbopol®, HPMC, Eudragit E100 or methyl cellulose. In one embodiment, the polymer is Carbopol®, HPMC or methyl cellulose. In one embodiment, the polymer is methyl cellulose. In another embodiment, the polymer is HPMC.

In one embodiment, the polymer is a cellulose ester polymer. In one embodiment, the polymer is cellulose phthalate (e.g., cellulose acetate phthalate or hydroxypropyl methyl cellulose phthalate (HPMC-P)) or cellulose succinate (e.g., hydroxylpropyl methyl cellulose succinate (HPMC-S) or hydroxypropyl methyl cellulose acetate succinate (HPMC-AS)). In one embodiment, the polymer is HPMC-P. In another embodiment, the polymer is HPMC-AS.

2. Solid Dispersions

In one embodiment, provided herein is a solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a pharmaceutically acceptable salt, polymorphs, solvates, hydrates, stereoisomers, prodrugs, or clathrates thereof (Compound 1) and a polymer. In one embodiment, the solid dispersion is amorphous.

In one embodiment, provided herein is a solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol (Compound 1A) and a polymer. In one embodiment, the solid dispersion is amorphous.

In some embodiments, provided herein is a solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate (Compound 1B) and a polymer. In one embodiment, the solid dispersion is amorphous.

In one embodiment, the solid dispersions provided herein comprise Compound 1 in an amount of from about 5% to about 75% and from about 5% to about 50%, from about 5%, 10%, 15%, 20%, 25%, 30%, 40%, or 50% by weight of the solid dispersion.

In one embodiment, the solid dispersions provided herein comprise Compound 1A in an amount of from about 5% to about 75% and from about 5% to about 50%, from about 5%, 10%, 15%, 20%, 25%, 30%, 40%, or 50% by weight of the solid dispersion. In one embodiment, the solid dispersions provided herein comprise Compound 1A in an amount of about 5%, 10%, 20%, 25% or 50% by weight of the solid dispersion. In one embodiment, the solid dispersion of Compound 1A is amorphous.

In one embodiment, the solid dispersions provided herein comprise Compound 1B in an amount of from about 5% to about 75% and from about 5% to about 50%, from about 5%, 10%, 15%, 20%, 25%, 30%, 40%, or 50% by weight of the solid dispersion. In one embodiment, the solid dispersions provided herein comprise Compound 1B in an amount of about 5%, 10%, 20%, 25% or 50% by weight of the solid dispersion. In one embodiment, the solid dispersion of Compound 1B is amorphous.

In one embodiment, provided herein is a process for preparing a solid dispersion comprising (a) providing a solution of Compound 1 and a polymer in a solvent; and (b) removing the solvent to provide the solid dispersion.

In one embodiment, the solvent comprises methanol, water, dichloromethane, tetrahydrofuran, acetone, trifluoroethanol, ethanol, or a mixture thereof. In one embodiment, the solvent is a mixture of acetone and methanol. In one embodiment, the solvent is a mixture of 80% acetone and 20% methanol by volume. In one embodiment, the solvent is a mixture of methanol and water. In one embodiment, the solvent is a mixture of 90% methanol and 10% water by volume. In one embodiment, the solvent is a mixture of tetrahydrofuran and water. In one embodiment, the solvent is a mixture of 50% tetrahydrofuran and 50% water by volume. In one embodiment, the solvent is a mixture of dichloromethane and methanol. In one embodiment, the solvent is a mixture of 50% dichloromethane and 50% methanol by volume. In one embodiment, the solvent is a mixture of dichloromethane and ethanol. In one embodiment, the solvent is a mixture of 50% dichloromethane and 50% ethanol by volume. In one embodiment, the solvent is a mixture of chloroform and ethanol. In one embodiment, the solvent is a mixture of 50% chloroform and 50% ethanol by volume. In one embodiment, the solvent is a mixture of trifluoroethanol and water. In one embodiment, the solvent is a mixture of 80% trifluoroethanol and 20% water by volume. In one embodiment, the solvent is a mixture of methanol and water. In one embodiment, the solvent is a mixture of 50% methanol and 50% water by volume.

In one embodiment, the solvent is removed by freeze evaporation. In one embodiment, the solvent is removed by spray drying.

In one embodiment, the solid dispersion provided herein comprises about 10% by weight of Compound 1 dispersed in Carbopol® 980 NF. In one embodiment, the solid dispersion comprise about 25% by weight of Compound 1 dispersed in Carbopol® 980 NF. In one embodiment, the solid dispersion comprise about 50% by weight of Compound 1 dispersed in Carbopol® 980 NF. In one embodiment, the solid dispersion is amorphous.

In one embodiment, the solid dispersion is obtained by freeze drying a solution of Compound 1 and Carbopol® 980 NF in a mixture of solvents methanol and water. In one embodiment, the ratio of methanol to water is about 90/10 v/v. In one embodiment, the solid dispersion is obtained by freeze drying a solution of Compound 1 and Carbopol® 980 NF in dichloromethane. In one embodiment, the solid dispersion is obtained by freeze drying a solution of Compound 1 and Carbopol® 980 NF in a mixture of solvents tetrahydrofuran and water. In one embodiment, the ratio of tetrahydrofuran to water is about 50/50 v/v. In one embodiment, the solid dispersion is obtained by freeze drying a solution of Compound 1 and Carbopol® 980 NF in a mixture of solvents trifluoroethanol and water. In one embodiment, the ratio of trifluoroethanol to water is about 80/20 v/v. In one embodiment, the ratio of trifluoroethanol to water is about 50/50 v/v. In one embodiment, the solid dispersion provided herein comprises Compound 1A. In one embodiment, the solid dispersion provided herein comprises Compound 1B. In one embodiment, the solid dispersion is amorphous.

In one embodiment, the solid dispersion is obtained by freeze drying a solution of Compound 1A and Carbopol® 980 NF in a mixture of solvents methanol and water. In one embodiment, the ratio of methanol to water is about 90/10 v/v. In one embodiment, the solid dispersion is obtained by freeze drying a solution of Compound 1A and Carbopol® 980 NF in dichloromethane. In one embodiment, the solid dispersion is obtained by freeze drying a solution of Compound 1A and Carbopol® 980 NF in a mixture of solvents tetrahydrofuran and water. In one embodiment, the ratio of tetrahydrofuran to water is about 50/50 v/v. In one embodiment, the solid dispersion is amorphous.

In one embodiment, the solid dispersion is obtained by freeze drying a solution of Compound 1B and Carbopol® 980 NF in a mixture of solvents trifluoroethanol and water. In one embodiment, the ratio of trifluoroethanol to water is about 80/20 v/v. In one embodiment, the ratio of trifluoroethanol to water is about 50/50 v/v. In one embodiment, the solid dispersion is obtained by freeze drying a solution of Compound 1B and Carbopol® 980 NF in a mixture of solvents methanol and water. In one embodiment, the ratio of methanol to water is about 90/10 v/v. In one embodiment, the solid dispersion is amorphous.

In one embodiment, the solid dispersion provided herein comprises about 10% by weight of Compound 1 dispersed in HPMC. In one embodiment, the solid dispersion comprise about 25% by weight of Compound 1 dispersed in HPMC. In one embodiment, the solid dispersion comprise about 50% by weight of Compound 1 dispersed in HPMC. In one embodiment, the solid dispersion is amorphous.

In one embodiment, the solid dispersion is obtained by freeze drying a solution of Compound 1 and HPMC in a mixture of solvents acetone and methanol. In one embodiment, the ratio of acetone to methanol is about 80/20 v/v. In one embodiment, the solid dispersion is obtained by freeze drying a solution of Compound 1 and HPMC in a mixture of solvents dichloromethane and ethanol. In one embodiment, the ratio of dichloromethane to ethanol is about 50/50 v/v. In one embodiment, the solid dispersion is obtained by freeze drying a solution of Compound 1 and HPMC in a mixture of solvents tetrahydrofuran and water. In one embodiment, the ratio of tetrahydrofuran to water is about 50/50 v/v. In one embodiment, the solid dispersion is obtained by freeze drying a solution of Compound 1 and HPMC in a mixture of solvents trifluoroethanol and water. In one embodiment, the ratio of trifluoroethanol to water is about 80/20 v/v. In one embodiment, the solid dispersion is obtained by freeze drying a solution of Compound 1 and HPMC in a mixture of solvents dichloromethane and methanol. In one embodiment, the ratio of dichloromethane to methanol is about 50/50 v/v. In one embodiment, the solid dispersion is obtained by freeze drying a solution of Compound 1 and HPMC in a mixture of solvents acetone and methanol. In one embodiment, the ratio of acetone to methanol is about 50/50 v/v. In one embodiment, the solid dispersion provided herein comprises Compound 1A. In one embodiment, the solid dispersion provided herein comprises Compound 1B. In one embodiment, the solid dispersion is amorphous.

In one embodiment, the solid dispersion is obtained by freeze drying a solution of Compound 1A and HPMC in a mixture of solvents acetone and methanol. In one embodiment, the ratio of acetone to methanol is about 80/20 v/v. In one embodiment, the solid dispersion is obtained by freeze drying a solution of Compound 1A and HPMC in a mixture of solvents dichloromethane and ethanol. In one embodiment, the ratio of dichloromethane to ethanol is about 50/50 v/v. In one embodiment, the solid dispersion is obtained by freeze drying a solution of Compound 1A and HPMC in a mixture of solvents tetrahydrofuran and water. In one embodiment, the ratio of tetrahydrofuran to water is about 50/50 v/v. In one embodiment, the solid dispersion is amorphous.

In one embodiment, the solid dispersion is obtained by freeze drying a solution of Compound 1B and HPMC in a mixture of solvents trifluoroethanol and water. In one embodiment, the ratio of trifluoroethanol to water is about 80/20 v/v. In one embodiment, the solid dispersion is obtained by freeze drying a solution of Compound 1B and HPMC in a mixture of solvents dichloromethane and methanol. In one embodiment, the ratio of dichloromethane to methanol is about 50/50 v/v. In one embodiment, the solid dispersion is obtained by freeze drying a solution of Compound 1B and HPMC in a mixture of solvents acetone and methanol. In one embodiment, the ratio of acetone to methanol is about 50/50 v/v. In one embodiment, the solid dispersion is amorphous.

In one embodiment, the solid dispersion provided herein comprises about 10% by weight of Compound 1 dispersed in methyl cellulose. In one embodiment, the solid dispersion comprises about 25% by weight of Compound 1 dispersed in methyl cellulose. In one embodiment, the solid dispersion comprises about 50% by weight of Compound 1 dispersed in methyl cellulose. In one embodiment, the solid dispersion is amorphous.

In one embodiment, the solid dispersion is obtained by freeze drying a solution of Compound 1 and methyl cellulose in a mixture of solvents dichloromethane and methanol. In one embodiment, the ratio of dichloromethane to methanol is about 50/50 v/v. In one embodiment, the solid dispersion is obtained by freeze drying a solution of Compound 1 and methyl cellulose in a mixture of solvents chloroform and ethanol. In one embodiment, the ratio of chloroform to ethanol is about 50/50 v/v. In one embodiment, the solid dispersion is obtained by freeze drying a solution of Compound 1 and methyl cellulose in a mixture of solvents tetrahydrofuran and water. In one embodiment, the ratio of tetrahydrofuran to water is about 50/50 v/v. In one embodiment, the solid dispersion is obtained by freeze drying a solution of Compound 1 and methyl cellulose in a mixture of solvents chloroform and ethanol. In one embodiment, the ratio of chloroform to ethanol is about 50/50 v/v. In one embodiment, the solid dispersion is obtained by freeze drying a solution of Compound 1 and methyl cellulose in a mixture of solvents trifluoroethanol and water. In one embodiment, the ratio of trifluoroethanol to water is about 80/20 v/v. In one embodiment, the solid dispersion is obtained by freeze drying a solution of Compound 1 and methyl cellulose in a mixture of solvents methanol and water. In one embodiment, the ratio of methanol to water is about 50/50 v/v. In one embodiment, the solid dispersion provided herein comprises Compound 1A. In one embodiment, the solid dispersion provided herein comprises Compound 1B. In one embodiment, the solid dispersion is amorphous.

In one embodiment, the solid dispersion is obtained by freeze drying a solution of Compound 1A and methyl cellulose in a mixture of solvents dichloromethane and methanol. In one embodiment, the ratio of dichloromethane to methanol is about 50/50 v/v. In one embodiment, the solid dispersion is obtained by freeze drying a solution of Compound 1A and methyl cellulose in a mixture of solvents chloroform and ethanol. In one embodiment, the ratio of chloroform to ethanol is about 50/50 v/v. In one embodiment, the solid dispersion is obtained by freeze drying a solution of Compound 1A and methyl cellulose in a mixture of solvents tetrahydrofuran and water. In one embodiment, the ratio of tetrahydrofuran to water is about 50/50 v/v. In one embodiment, the solid dispersion is amorphous.

In one embodiment, the solid dispersion is obtained by freeze drying a solution of Compound 1B and methyl cellulose in a mixture of solvents chloroform and ethanol. In one embodiment, the ratio of chloroform to ethanol is about 50/50 v/v. In one embodiment, the solid dispersion is obtained by freeze drying a solution of Compound 1B and methyl cellulose in a mixture of solvents trifluoroethanol and water. In one embodiment, the ratio of trifluoroethanol to water is about 80/20 v/v. In one embodiment, the solid dispersion is obtained by freeze drying a solution of Compound 1B and methyl cellulose in a mixture of solvents methanol and water. In one embodiment, the ratio of methanol to water is about 50/50 v/v. In one embodiment, the solid dispersion is amorphous.

Exemplary Amorphous Solid Dispersions

In one embodiment, provided herein is an amorphous solid dispersion comprising Compound 1 dispersed in a solid matrix comprising a polymer. In one embodiment, provided herein is an amorphous solid dispersion comprising Compound 1A dispersed in a solid matrix comprising a polymer. In one embodiment, provided herein is an amorphous solid dispersion comprising Compound 1B dispersed in a solid matrix comprising a polymer.

In one embodiment, provided herein is an amorphous solid dispersion comprising about 50% by weight of Compound 1A, dispersed in a solid matrix that comprises Carbopol® 980 NF. In one embodiment, the amorphous solid dispersion is obtained by freeze drying a solution of Compound 1A, and Carbopol® 980 NF in a mixture of methanol and water. In one embodiment, the ratio of methanol and water is about 90/10 v/v.

In one embodiment, the amorphous solid dispersion exhibits a mass loss of about 5.2%, as determined by TGA, upon heating from about 25° C. to about 300° C. Without being limited by a particular theory, the mass loss corresponds to loss of residue solvent mixture methanol and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 10% by weight of Compound 1A, dispersed in a solid matrix that comprises Carbopol® 980 NF. In one embodiment, the amorphous solid dispersion is obtained by freeze drying a solution of Compound 1A, and Carbopol® 980 NF in dichloromethane.

In one embodiment, the amorphous solid dispersion exhibits a mass loss of about 7.1%, as determined by TGA, upon heating from about 25° C. to about 300° C. Without being limited by a particular theory, the mass loss corresponds to loss of residue solvent of dichloromethane. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid (SGF) or simulated intestinal fluid (SIF).

In one embodiment, provided herein is an amorphous solid dispersion comprising about 25% by weight of Compound 1A, dispersed in a solid matrix that comprises Carbopol® 980 NF. In one embodiment, the amorphous solid dispersion is obtained by freeze drying a solution of Compound 1A, and Carbopol® 980 NF in dichloromethane.

In one embodiment, the amorphous solid dispersion exhibits a mass loss of about 5.7%, as determined by TGA, upon heating from about 25° C. to about 300° C. Without being limited by a particular theory, the mass loss corresponds to loss of residue solvent dichloromethane. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 10% by weight of Compound 1A, dispersed in a solid matrix that comprises Carbopol® 980 NF. In one embodiment, the amorphous solid dispersion is obtained by freeze drying a solution of Compound 1A, and Carbopol® 980 NF in a mixture of tetrahydrofuran and water. In one embodiment, the ratio of tetrahydrofuran and water is about 50/50 v/v.

In one embodiment, the amorphous solid dispersion exhibits a mass loss of about 5.2%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 25% by weight of Compound 1A, dispersed in a solid matrix that comprises Carbopol® 980 NF. In one embodiment, the amorphous solid dispersion is obtained by freeze drying a solution of Compound 1A, and Carbopol® 980 NF in a mixture of tetrahydrofuran and water. In one embodiment, the ratio of tetrahydrofuran and water is about 50/50 v/v.

In one embodiment, the amorphous solid dispersion exhibits a mass loss of about 5.7%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 50% by weight of Compound 1A, dispersed in a solid matrix that comprises Carbopol® 980 NF. In one embodiment, the amorphous solid dispersion is obtained by freeze drying a solution of Compound 1A, and Carbopol® 980 NF in a mixture of tetrahydrofuran and water. In one embodiment, the ratio of tetrahydrofuran and water is about 50/50 v/v.

In one embodiment, the amorphous solid dispersion exhibits a mass loss of about 4.9%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 10% by weight of Compound 1A, dispersed in a solid matrix that comprises HPMC. In one embodiment, the amorphous solid dispersion is obtained by freeze drying a solution of Compound 1A, and HPMC in a mixture of acetone and methanol. In one embodiment, the ratio of acetone and methanol is about 80/20 v/v. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 50% by weight of Compound 1A, dispersed in a solid matrix that comprises HPMC. In one embodiment, the amorphous solid dispersion is obtained by freeze drying a solution of Compound 1A, and HPMC in a mixture of acetone and methanol. In one embodiment, the ratio of acetone and methanol is about 80/20 v/v.

In one embodiment, the amorphous solid dispersion exhibits a mass loss of about 3.6%, as determined by TGA, upon heating from about 25° C. to about 300° C. Without being limited by a particular theory, the mass loss corresponds to loss of residue solvent mixture acetone and methanol. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 10% by weight of Compound 1A, dispersed in a solid matrix that comprises HPMC. In one embodiment, the amorphous solid dispersion is obtained by freeze drying a solution of Compound 1A, and HPMC in a mixture of tetrahydrofuran and water. In one embodiment, the ratio of acetone and methanol is about 50/50 v/v.

In one embodiment, the amorphous solid dispersion exhibits a mass loss of about 4.1%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 25% by weight of Compound 1A, dispersed in a solid matrix that comprises HPMC. In one embodiment, the amorphous solid dispersion is obtained by freeze drying a solution of Compound 1A, and HPMC in a mixture of tetrahydrofuran and water. In one embodiment, the ratio of acetone and methanol is about 50/50 v/v.

In one embodiment, the amorphous solid dispersion exhibits a mass loss of about 3.1%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 50% by weight of Compound 1A, dispersed in a solid matrix that comprises HPMC. In one embodiment, the amorphous solid dispersion is obtained by freeze drying a solution of Compound 1A, and HPMC in a mixture of tetrahydrofuran and water. In one embodiment, the ratio of acetone and methanol is about 50/50 v/v. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 25% by weight of Compound 1A, dispersed in a solid matrix that comprises methyl cellulose. In one embodiment, the amorphous solid dispersion is obtained by freeze drying a solution of Compound 1A, and methyl cellulose in a mixture of chloroform and ethanol. In one embodiment, the ratio of chloroform and ethanol is about 50/50 v/v.

In one embodiment, the amorphous solid dispersion exhibits a mass loss of about 3.9%, as determined by TGA, upon heating from about 25° C. to about 300° C. Without being limited by a particular theory, the mass loss corresponds to loss of residue solvent mixture chloroform and ethanol. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 10% by weight of Compound 1A, dispersed in a solid matrix that comprises methyl cellulose. In one embodiment, the amorphous solid dispersion is obtained by freeze drying a solution of Compound 1A, and methyl cellulose in a mixture of tetrahydrofuran and water. In one embodiment, the ratio of acetone and methanol is about 50/50 v/v.

In one embodiment, the amorphous solid dispersion exhibits a mass loss of about 4.1%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 25% by weight of Compound 1A, dispersed in a solid matrix that comprises methyl cellulose. In one embodiment, the amorphous solid dispersion is obtained by freeze drying a solution of Compound 1A, and methyl cellulose in a mixture of tetrahydrofuran and water. In one embodiment, the ratio of acetone and methanol is about 50/50 v/v. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 50% by weight of Compound 1A, dispersed in a solid matrix that comprises methyl cellulose. In one embodiment, the amorphous solid dispersion is obtained by freeze drying a solution of Compound 1A, and methyl cellulose in a mixture of tetrahydrofuran and water. In one embodiment, the ratio of acetone and methanol is about 50/50 v/v.

In one embodiment, the amorphous solid dispersion exhibits a mass loss of about 3.7%, as determined by TGA, upon heating from about 25° C. to about 300° C. Without being limited by a particular theory, the mass loss corresponds to loss of residue solvent mixture tetrahydrofuran and water. In one embodiment, the amorphous solid dispersion exhibits no melting event between about 25° C. and about 350° C., as determined by DSC. In one embodiment, the amorphous solid dispersion remains amorphous upon exposure to about 40° C./70% RH for four weeks. In one embodiment, the amorphous solid dispersion exhibits an increased solubility of Compound 1A in an aqueous media as compared to neat Compound 1A. In one embodiment, the aqueous media is water, simulated gastric fluid or simulated intestinal fluid.

In one embodiment, provided herein is an amorphous solid dispersion comprising about 10% by weight of Compound 1B, dispersed in a solid matrix that comprises Carbopol® 980 NF. In one embodiment, the amorphous solid dispersion is obtained by freeze drying a solution of Compound 1B, and Carbopol® 980 NF in tetrahydrofuran and water. In one embodiment, the ratio of tetrahydrofuran and water is about 50/50 v/v.

In one embodiment, the amorphous solid dispersion exhibits a mass loss of about 1.9%, as determined by TGA, upon heating from about 25° C. to about 300° C. Without being limited by a particular theory, the mass loss corresponds to loss of residue solvent mixture trifluoroethanol and water. In one embodiment, the amorphous solid dispersion exhibits no melting event between about 25° C. and about 350° C., as determined by DSC. Thermal decomposition was observed after 180° C. In one embodiment, the amorphous solid dispersion remains amorphous upon exposure to about 40° C./70% RH for four weeks. In one embodiment, the amorphous solid dispersion exhibits an increased solubility of Compound 1B in an aqueous media as compared to neat Compound 1B. In one embodiment, the aqueous media is water, simulated gastric fluid or simulated intestinal fluid.

In one embodiment, provided herein is an amorphous solid dispersion comprising about 25% by weight of Compound 1B, dispersed in a solid matrix that comprises HPMC. In one embodiment, the amorphous solid dispersion is obtained by freeze drying a solution of Compound 1B, and HPMC in trifluoroethanol and water. In one embodiment, the ratio of trifluoroethanol and water is about 80/20 v/v.

In one embodiment, the amorphous solid dispersion exhibits a mass loss of about 4.8%, as determined by TGA, upon heating from about 25° C. to about 300° C. Without being limited by a particular theory, the mass loss corresponds to loss of residue solvent mixture trifluoroethanol and water. In one embodiment, the amorphous solid dispersion exhibits no melting event between about 25° C. and about 350° C., as determined by DSC. Thermal decomposition was observed after 180° C. In one embodiment, the amorphous solid dispersion remains amorphous upon exposure to about 40° C./70% RH for four weeks. In one embodiment, the amorphous solid dispersion exhibits an increased solubility of Compound 1B in an aqueous media as compared to neat Compound 1B. In one embodiment, the aqueous media is water, simulated gastric fluid or simulated intestinal fluid.

In one embodiment, provided herein is an amorphous solid dispersion comprising about 10% by weight of Compound 1B, dispersed in a solid matrix that comprises methyl cellulose. In one embodiment, the amorphous solid dispersion is obtained by freeze drying a solution of Compound 1B, and methyl cellulose in chloroform and ethanol. In one embodiment, the ratio of chloroform and ethanol is about 50/50 v/v.

In one embodiment, the amorphous solid dispersion exhibits a mass loss of about 6.8%, as determined by TGA, upon heating from about 25° C. to about 300° C. Without being limited by a particular theory, the mass loss corresponds to loss of residue solvent mixture chloroform and ethanol. In one embodiment, the amorphous solid dispersion exhibits no melting event between about 25° C. and about 350° C., as determined by DSC. Thermal decomposition was observed after 180° C. In one embodiment, the amorphous solid dispersion remains amorphous upon exposure to about 40° C./70% RH for four weeks. In one embodiment, the amorphous solid dispersion exhibits an increased solubility of Compound 1B in an aqueous media as compared to neat Compound 1B. In one embodiment, the aqueous media is water, simulated gastric fluid or simulated intestinal fluid.

In certain embodiments, the amorphous solid dispersion provided herein is physically stable. In certain embodiments, the amorphous solid dispersion provided herein can be prepared with a high drug load. In certain embodiments, the amorphous solid dispersion provided herein gives increased solubility of the API in gastric simulated fluid. In certain embodiments, the amorphous solid dispersion provided herein gives increased solubility of the API in intestinal simulated fluid. In certain embodiments, the amorphous solid dispersion provided herein gives increased solubility of the API in both gastric simulated fluid and intestinal simulated fluid. In one embodiment, the API is Compound 1. In one embodiment, the API is Compound 1A. In one embodiment, the API is Compound 1B. In one embodiment, the anti-precipitation capacity of HPMC resulted in a prolonged supersaturated concentration of Compound 1A in solution which may result in increased bioavailability of the drug when administered as an amorphous solid dispersion.

3. Compositions Containing the Solid Dispersions and Routes of Administration

In one embodiment, the solid dispersions provided herein are formulated with a pharmaceutically acceptable carrier or adjuvant into pharmaceutically acceptable compositions prior to be administered to a subject. In another embodiment, such pharmaceutically acceptable compositions further comprise additional therapeutic agents in amounts effective for achieving a modulation of disease or disease symptoms, including those described herein.

Pharmaceutically acceptable carriers, adjuvants and vehicles that may be used in the pharmaceutical compositions of one aspect of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as TWEENs or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the solid dispersions described herein.

In one embodiment, the pharmaceutical composition comprises a solid dispersion and an excipient. In one embodiment, the pharmaceutical composition that comprises a solid dispersion and an excipient, is for oral administration. In one embodiment, the excipient is a diluent, a binder, a disintegrant, a wetting agent, a stabilizer, a glidant, or a lubricant.

In one embodiment, the diluent is a microcrystalline cellulose.

In one embodiment, the binder is a hydroxypropyl cellulose.

In one embodiment, the disintegrant is sodium starch glycolate.

In one embodiment, the wetting agent is sodium lauryl sulfate.

In one embodiment, the stabilizer is hypromellose acetate succinate.

In one embodiment, the glidant is colloidal silicon dioxide.

In one embodiment, the lubricant is magnesium stearate.

Oral delivery formats include, but are not limited to, tablets, capsules, caplets, solutions, suspensions, and syrups, and may also comprise a plurality of granules, beads, powders or pellets that may or may not be encapsulated. Such formats may also be referred to herein as the “drug core” which contains a solid dispersion provided herein.

Particular embodiments herein provide solid oral dosage forms that are tablets or capsules. In certain embodiments, the formulation is a tablet comprising a solid dispersion provided herein. In certain embodiments, the formulation is a capsule comprising a solid dispersion provided herein. In certain embodiments, the tablets or capsules provided herein optionally comprise one or more excipients, such as, for example, glidants, diluents, lubricants, colorants, disintegrants, granulating agents, binding agents, polymers, and coating agents. In certain embodiments, the formulation is an immediate release tablet. In certain embodiments, the formulation is a controlled release tablet releasing the active pharmaceutical ingredient (API), e.g., substantially in the stomach. In certain embodiments, the formulation is a hard gelatin capsule. In certain embodiments, the formulation is a soft gelatin capsule. In certain embodiments, the capsule is a hydroxypropyl methylcellulose (HPMC) capsule. In certain embodiments, the formulation is an immediate release capsule. In certain embodiments, the formulation is an immediate or controlled release capsule releasing the API, e.g., substantially in the stomach. In certain embodiments, the formulation is a rapidly disintegrating tablet that dissolves substantially in the mouth following administration. In one embodiment, the API is Compound 1. In one embodiment, the API is Compound 1A. In one embodiment, the API is Compound 1B.

Particular embodiments herein provide pharmaceutical formulations (e.g., immediate release oral formulations and/or formulations that release the API substantially in the stomach) comprising a solid dispersion provided herein that achieve a particular AUC value (e.g., AUC(0-t) or AUC(0-∞)) in the subject (e.g., human) to which the formulation is orally administered. In one embodiment, the API is Compound 1. In one embodiment, the API is Compound 1A. In one embodiment, the API is Compound 1B. Particular embodiments provide oral formulations that achieve an AUC value of at least about 25 ng-hr/mL, at least about 50 ng-hr/mL, at least about 75 ng-hr/mL, at least about 100 ng-hr/mL, at least about 150 ng-hr/mL, at least about 200 ng-hr/mL, at least about 250 ng-hr/mL, at least about 300 ng-hr/mL, at least about 350 ng-hr/mL, at least about 400 ng-hr/mL, at least about 450 ng-hr/mL, at least about 500 ng-hr/mL, at least about 550 ng-hr/mL, at least about 600 ng-hr/mL, at least about 650 ng-hr/mL, at least about 700 ng-hr/mL, at least about 750 ng-hr/mL, at least about 800 ng-hr/mL, at least about 850 ng-hr/mL, at least about 900 ng-hr/mL, at least about 950 ng-hr/mL, at least about 1000 ng-hr/mL, at least about 1100 ng-hr/mL, at least about 1200 ng-hr/mL, at least about 1300 ng-hr/mL, at least about 1400 ng-hr/mL, at least about 1500 ng-hr/mL, at least about 1600 ng-hr/mL, at least about 1700 ng-hr/mL, at least about 1800 ng-hr/mL, at least about 1900 ng-hr/mL, at least about 2000 ng-hr/mL, at least about 2250 ng-hr/mL, or at least about 2500 ng-hr/mL. In particular embodiments, the AUC determination is obtained from a time-concentration pharmacokinetic profile obtained from the blood samples of animals or human volunteers following dosing.

Particular embodiments herein provide pharmaceutical formulations (e.g., immediate release oral formulations and/or formulations that release the API substantially in the stomach) comprising a solid dispersion provided herein that achieve a particular maximum plasma concentration (“Cmax”) in the subject to which the formulation is orally administered. In one embodiment, the API is Compound 1. In one embodiment, the API is Compound 1A. In one embodiment, the API is Compound 1B. Particular embodiments provide oral formulations that achieve a Cmax of Compound 1 of at least about 25 ng/mL, at least about 50 ng/mL, at least about 75 ng/mL, at least about 100 ng/mL, at least about 150 ng/mL, at least about 200 ng/mL, at least about 250 ng/mL, at least about 300 ng/mL, at least about 350 ng/mL, at least about 400 ng/mL, at least about 450 ng/mL, at least about 500 ng/mL, at least about 550 ng/mL, at least about 600 ng/mL, at least about 650 ng/mL, at least about 700 ng/mL, at least about 750 ng/mL, at least about 800 ng/mL, at least about 850 ng/mL, at least about 900 ng/mL, at least about 950 ng/mL, at least about 1000 ng/mL, at least about 1100 ng/mL, at least about 1200 ng/mL, at least about 1300 ng/mL, at least about 1400 ng/mL, at least about 1500 ng/mL, at least about 1600 ng/mL, at least about 1700 ng/mL, at least about 1800 ng/mL, at least about 1900 ng/mL, at least about 2000 ng/mL, at least about 2250 ng/mL, or at least about 2500 ng/mL.

Particular embodiments herein provide pharmaceutical formulations (e.g., immediate release oral formulations and/or formulations that release the API substantially in the stomach) comprising a solid dispersion provided herein that achieve a particular time to maximum plasma concentration (“Tmax”) in the subject to which the formulation is orally administered. In one embodiment, the API is Compound 1. In one embodiment, the API is Compound 1A. In one embodiment, the API is Compound 1B. Particular embodiments provide oral formulations that achieve a Tmax of Compound 1 of less than about 10 min., less than about 15 min., less than about 20 min., less than about 25 min., less than about 30 min., less than about 35 min., less than about 40 min., less than about 45 min., less than about 50 min., less than about 55 min., less than about 60 min., less than about 65 min., less than about 70 min., less than about 75 min., less than about 80 min., less than about 85 min., less than about 90 min., less than about 95 min., less than about 100 min., less than about 105 min., less than about 110 min., less than about 115 min., less than about 120 min., less than about 130 min., less than about 140 min., less than about 150 min., less than about 160 min., less than about 170 min., less than about 180 min., less than about 190 min., less than about 200 min., less than about 210 min., less than about 220 min., less than about 230 min., or less than about 240 min. In particular embodiments, the Tmax value is measured from the time at which the formulation is orally administered.

Particular embodiments herein provide oral dosage forms comprising a solid dispersion provided herein wherein the oral dosage forms have an enteric coating. Particular embodiments provide a permeable or partly permeable (e.g., “leaky”) enteric coating with pores. In particular embodiments, the permeable or partly permeable enteric-coated tablet releases Compound 1 in an immediate release manner substantially in the stomach.

Provided herein are dosage forms designed to maximize the absorption and/or efficacious delivery of Compound 1, upon oral administration, e.g., for release substantially in the stomach. Accordingly, certain embodiments herein provide a solid oral dosage form comprising a solid dispersion provided herein using pharmaceutical excipients designed for immediate release of the API upon oral administration, e.g., substantially in the stomach. In one embodiment, the API is Compound 1. In one embodiment, the API is Compound 1A. In one embodiment, the API is Compound 1B. Particular immediate release formulations comprise a specific amount of a solid dispersion provided herein and optionally one or more excipients. In certain embodiments, the formulation may be an immediate release tablet or an immediate release capsule (such as, e.g., an HPMC capsule).

Provided herein are methods of making the formulations provided herein comprising a solid dispersion provided herein provided herein (e.g., immediate release oral formulations and/or formulations that release the API substantially in the stomach). In one embodiment, the API is Compound 1. In one embodiment, the API is Compound 1A. In one embodiment, the API is Compound 1B. In particular embodiments, the formulations provided herein may be prepared using conventional methods known to those skilled in the field of pharmaceutical formulation, as described, e.g., in pertinent textbooks. See, e.g., REMINGTON, THE SCIENCE AND PRACTICE OF PHARMACY, 20th Edition, Lippincott Williams & Wilkins, (2000); ANSEL et al., PHARMACEUTICAL DOSAGE FORMS AND DRUG DELIVERY SYSTEMS, 7th Edition, Lippincott Williams & Wilkins, (1999); GIBSON, PHARMACEUTICAL PREFORMULATION AND FORMULATION, CRC Press (2001).

In particular embodiments, formulations provided herein (e.g., immediate release oral formulations, formulations that release the API substantially in the stomach, or rapidly disintegrating formulations that dissolve substantially in the mouth) comprise a solid dispersion provided herein in a specific amount. In one embodiment, the API is Compound 1. In one embodiment, the API is Compound 1A. In one embodiment, the API is Compound 1B. In particular embodiments, the specific amount of a solid dispersion provided in the formulation is, e.g., about 10 mg. In one embodiment, the specific amount is about 20 mg. In one embodiment, the specific amount is about 40 mg. In one embodiment, the specific amount is about 60 mg. In one embodiment, the specific amount is about 80 mg. In one embodiment, the specific amount is about 100 mg. In one embodiment, the specific amount is about 120 mg. In one embodiment, the specific amount is about 140 mg. In one embodiment, the specific amount is about 150 mg. In one embodiment, the specific amount is about 160 mg. In one embodiment, the specific amount is about 180 mg. In one embodiment, the specific amount is about 200 mg. In one embodiment, the specific amount is about 220 mg. In one embodiment, the specific amount is about 240 mg. In one embodiment, the specific amount is about 260 mg. In one embodiment, the specific amount is about 280 mg. In one embodiment, the specific amount is about 300 mg. In one embodiment, the specific amount is about 320 mg. In one embodiment, the specific amount is about 340 mg. In one embodiment, the specific amount is about 360 mg. In one embodiment, the specific amount is about 380 mg. In one embodiment, the specific amount is about 400 mg. In one embodiment, the specific amount is about 420 mg. In one embodiment, the specific amount is about 440 mg. In one embodiment, the specific amount is about 460 mg. In one embodiment, the specific amount is about 480 mg. In one embodiment, the specific amount is about 500 mg. In one embodiment, the specific amount is about 600 mg. In one embodiment, the specific amount is about 700 mg. In one embodiment, the specific amount is about 800 mg. In one embodiment, the specific amount is about 900 mg. In one embodiment, the specific amount is about 1000 mg. In one embodiment, the specific amount is about 1100 mg. In one embodiment, the specific amount is about 1200 mg. In one embodiment, the specific amount is about 1300 mg. In one embodiment, the specific amount is about 1400 mg. In one embodiment, the specific amount is about 1500 mg. In one embodiment, the specific amount is about 1600 mg. In one embodiment, the specific amount is about 1700 mg. In one embodiment, the specific amount is about 1800 mg. In one embodiment, the specific amount is about 1900 mg. In one embodiment, the specific amount is about 2000 mg. In one embodiment, the specific amount is about 2100 mg. In one embodiment, the specific amount is about 2200 mg. In one embodiment, the specific amount is about 2300 mg. In one embodiment, the specific amount is about 2400 mg. In one embodiment, the specific amount is about 2500 mg. In one embodiment, the specific amount is about 3000 mg. In one embodiment, the specific amount is about 4000 mg. In one embodiment, the specific amount is about 5000 mg.

In certain embodiments, the formulation is a tablet, wherein the tablet is manufactured using standard, art-recognized tablet processing procedures and equipment. In certain embodiments, the method for forming the tablets is direct compression of a powdered, crystalline and/or granular composition comprising a solid dispersion provided herein alone or in combination with one or more excipients, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the formulation is a capsule, wherein the capsules may be manufactured using standard, art-recognized capsule processing procedures and equipments. In certain embodiments, soft gelatin capsules may be prepared in which the capsules contain a mixture of a solid dispersion provided herein and vegetable oil or non-aqueous, water miscible materials such as, for example, polyethylene glycol and the like. In certain embodiments, hard gelatin capsules may be prepared containing granules of a solid dispersion provided herein in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

In certain embodiments, the formulation of a solid dispersion provided herein is prepared using aqueous solvents without causing significant hydrolytic degradation of the compound. In particular embodiments, the formulation of a solid dispersion provided herein is a tablet which contains a coating applied to the drug core using aqueous solvents without causing significant hydrolytic degradation of the compound in the formulation. In certain embodiments, water is employed as the solvent for coating the drug core. In certain embodiments, the oral dosage form of a solid dispersion provided herein is a tablet containing a film coat applied to the drug core using aqueous solvents. In particular embodiments, water is employed as the solvent for film-coating. In particular embodiments, the tablet containing a solid dispersion provided herein is film-coated using aqueous solvents without effecting degradation of the pharmaceutical composition. In particular embodiments, water is used as the film coating solvent without effecting degradation of the pharmaceutical composition. In certain embodiments, an oral dosage form comprising a solid dispersion provided herein and an aqueous film coating effects immediate drug release upon oral delivery. In certain embodiments, the oral dosage form comprising a solid dispersion provided herein and an aqueous film coating effects controlled drug release to the upper gastrointestinal tract, e.g., the stomach, upon oral administration. In particular embodiments, a tablet with an aqueous-based film coating comprises Compound 1 as the API. In one embodiment, a tablet with an aqueous-based film coating comprises Compound 1A as the API. In one embodiment, a tablet with an aqueous-based film coating comprises Compound 1B as the API.

In certain embodiments, provided herein is a controlled release pharmaceutical formulation for oral administration of a solid dispersion provided herein, wherein the release occurs substantially in the stomach, comprising: a) a specific amount of a solid dispersion provided herein; b) a drug release controlling component for controlling the release of a solid dispersion provided herein substantially in the upper gastrointestinal tract, e.g., the stomach; and c) optionally one or more excipients. In certain embodiments, the oral dosage form comprising a solid dispersion provided herein is prepared as a controlled release tablet or capsule which includes a drug core comprising the pharmaceutical composition and optional excipients. Optionally, a “seal coat” or “shell” is applied. In certain embodiments, a formulation provided herein comprising a solid dispersion provided herein is a controlled release tablet or capsule, which comprises a therapeutically effective amount of a solid dispersion provided herein, a drug release controlling component that controls the release of Compound 1 substantially in the stomach upon oral administration, and optionally, one or more excipients.

Particular embodiments provide a drug release controlling component that is a polymer matrix, which swells upon exposure to gastric fluid to effect the gastric retention of the formulation and the sustained release of Compound 1 from the polymer matrix substantially in the stomach.

In certain embodiments, the drug release controlling component may comprise a shell surrounding the drug-containing core, wherein the shell releases Compound 1 from the core by, e.g., permitting diffusion of Compound 1 from the core and promoting gastric retention of the formulation by swelling upon exposure to gastric fluids to a size that is retained in the stomach. In certain embodiments, such formulations may be prepared by first compressing a mixture of a solid dispersion provided herein and one or more excipients to form a drug core, and compressing another powdered mixture over the drug core to form the shell, or enclosing the drug core with a capsule shell made of suitable materials. Examples of such formulations are known in the art. See, e.g., Berner et al., U.S. Patent Publication No. 2003/0104062 application Ser. No. 10/213,823), incorporated herein by reference in its entirety.

In certain embodiments, the pharmaceutical formulations provided herein contain a solid dispersion provided herein and, optionally, one or more excipients to form a “drug core.” Optional excipients include, e.g., diluents (bulking agents), lubricants, disintegrants, fillers, stabilizers, surfactants, preservatives, coloring agents, flavoring agents, binding agents, excipient supports, glidants, permeation enhancement excipients, plasticizers and the like, e.g., as known in the art. It will be understood by those in the art that some substances serve more than one purpose in a pharmaceutical composition. For instance, some substances are binders that help hold a tablet together after compression, yet are also disintegrants that help break the tablet apart once it reaches the target delivery site. Selection of excipients and amounts to use may be readily determined by the formulation scientist based upon experience and consideration of standard procedures and reference works available in the art.

In certain embodiments, formulations provided herein comprise one or more binders. Binders may be used, e.g., to impart cohesive qualities to a tablet, and thus ensure that the tablet remains intact after compression. Suitable binders include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropylmethylcellulose, methyl cellulose, ethyl cellulose, hydroxyethyl cellulose, carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein. The binding agent can be, relative to the drug core, in the amount of about 2% w/w of the drug core; about 4% w/w of the drug core, about 6% w/w of the drug core, about 8% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, about 34% w/w of the drug core, about 36% w/w of the drug core, about 38% w/w of the drug core, about 40% w/w of the drug core, about 42% w/w of the drug core, about 44% w/w of the drug core, about 46% w/w of the drug core, about 48% w/w of the drug core, about 50% w/w of the drug core, about 52% w/w of the drug core, about 54% w/w of the drug core, about 56% w/w of the drug core, about 58% w/w of the drug core, about 60% w/w of the drug core, about 62% w/w of the drug core, about 64% w/w of the drug core, about 66% w/w of the drug core; about 68% w/w of the drug core, about 70% w/w of the drug core, about 72% w/w of the drug core, about 74% w/w of the drug core, about 76% w/w of the drug core, about 78% w/w of the drug core, about 80% w/w of the drug core, about 82% w/w of the drug core, about 84% w/w of the drug core, about 86% w/w of the drug core, about 88% w/w of the drug core, about 90% w/w of the drug core, about 92% w/w of the drug core, about 94% w/w of the drug core, about 96% w/w of the drug core, about 98% w/w of the drug core, or more, if determined to be appropriate. In certain embodiments, a suitable amount of a particular binder is determined by one of ordinary skill in the art.

In certain embodiments, formulations provided herein comprise one or more diluents. Diluents may be used, e.g., to increase bulk so that a practical size tablet is ultimately provided. Suitable diluents include dicalcium phosphate, calcium sulfate, lactose, cellulose, kaolin, mannitol, sodium chloride, dry starch, microcrystalline cellulose (e.g., AVICEL), microtine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-β-cyclodextrin, talc, tragacanth, trehalose, and xylitol. Diluents may be used in amounts calculated to obtain a desired volume for a tablet or capsule; in certain embodiments, a diluent is used in an amount of about 5% or more, about 10% or more, about 15% or more, about 20% or more, about 22% or more, about 24% or more, about 26% or more, about 28% or more, about 30% or more, about 32% or more, about 34% or more, about 36% or more, about 38% or more, about 40% or more, about 42% or more, about 44% or more, about 46% or more, about 48% or more, about 50% or more, about 52% or more, about 54% or more, about 56% or more, about 58% or more, about 60% or more, about 62% or more, about 64% or more, about 68% or more, about 70% ore more, about 72% or more, about 74% or more, about 76% or more, about 78% or more, about 80% or more, about 85% or more, about 90% or more, or about 95% or more, weight/weight, of a drug core; between about 10% and about 90% w/w of the drug core; between about 20% and about 80% w/w of the drug core; between about 30% and about 70% w/w of the drug core; between about 40% and about 60% w/w of the drug core. In certain embodiments, a suitable amount of a particular diluent is determined by one of ordinary skill in the art.

In certain embodiments, formulations provided herein comprise one or more lubricants. Lubricants may be used, e.g., to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. In certain embodiments, stearates, if present, represent no more than approximately 2 weight % of the drug-containing core. Further examples of lubricants include, e.g., calcium stearate, glycerin monostearate, glyceryl behenate, glyceryl palmitostearate, magnesium lauryl sulfate, magnesium stearate, myristic acid, palmitic acid, poloxamer, polyethylene glycol, potassium benzoate, sodium benzoate, sodium chloride, sodium lauryl sulfate, sodium stearyl fumarate, stearic acid, talc, and zinc stearate. In particular embodiments, the lubricant is magnesium stearate. In certain embodiments, the lubricant is present, relative to the drug core, in an amount of about 0.2% w/w of the drug core, about 0.4% w/w of the drug core, about 0.6% w/w of the drug core, about 0.8% w/w of the drug core, about 1.0% w/w of the drug core, about 1.2% w/w of the drug core, about 1.4% w/w of the drug core, about 1.6% w/w of the drug core, about 1.8% w/w of the drug core, about 2.0% w/w of the drug core, about 2.2% w/w of the drug core, about 2.4% w/w of the drug core, about 2.6% w/w of the drug core, about 2.8% w/w of the drug core, about 3.0% w/w of the drug core, about 3.5% w/w of the drug core, about 4% w/w of the drug core, about 4.5% w/w of the drug core, about 5% w/w of the drug core, about 6% w/w of the drug core, about 7% w/w of the drug core, about 8% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 25% w/w of the drug core, about 30% w/w of the drug core, about 35% w/w of the drug core, about 40% w/w of the drug core, between about 0.2% and about 10% w/w of the drug core, between about 0.5% and about 5% w/w of the drug core, or between about 1% and about 3% w/w of the drug core. In certain embodiments, a suitable amount of a particular lubricant is determined by one of ordinary skill in the art.

In certain embodiments, formulations provided herein comprise one or more disintegrants. Disintegrants may be used, e.g., to facilitate disintegration of the tablet, and may be, e.g., starches, clays, celluloses, algins, gums or crosslinked polymers. Disintegrants also include, e.g., alginic acid, carboxymethylcellulose calcium, carboxymethylcellulose sodium (e.g., AC-DI-SOL, PRIMELLOSE), colloidal silicon dioxide, croscarmellose sodium, crospovidone (e.g., KOLLIDON, POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB) and starch. Additional disintegrants include, e.g., calcium alginate, chitosan, sodium docusate, hydroxypropyl cellulose, and povidone. In certain embodiments, the disintegrant is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, greater than about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular disintegrant is determined by one of ordinary skill in the art.

In certain embodiments, formulations provided herein comprise one or more stabilizers. Stabilizers (also called absorption enhancers) may be used, e.g., to inhibit or retard drug decomposition reactions that include, by way of example, oxidative reactions. Stabilizing agents include, e.g., d-alpha-tocopheryl polyethylene glycol 1000 succinate (Vitamin E TPGS), acacia, albumin, alginic acid, aluminum stearate, ammonium alginate, ascorbic acid, ascorbyl palmitate, bentonite, butylated hydroxytoluene, calcium alginate, calcium stearate, calcium carboxymethylcellulose, carrageenan, ceratonia, colloidal silicon dioxide, cyclodextrins, diethanolamine, edetates, ethylcellulose, ethyleneglycol palmitostearate, glycerin monostearate, guar gum, hydroxypropyl cellulose, hypromellose, invert sugar, lecithin, magnesium aluminum silicate, monoethanolamine, pectin, poloxamer, polyvinyl alcohol, potassium alginate, potassium polacrilin, povidone, propyl gallate, propylene glycol, propylene glycol alginate, raffinose, sodium acetate, sodium alginate, sodium borate, sodium carboxymethyl cellulose, sodium stearyl fumarate, sorbitol, stearyl alcohol, sufobutyl-b-cyclodextrin, trehalose, white wax, xanthan gum, xylitol, yellow wax, and zinc acetate. In certain embodiments, the stabilizer is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular stabilizer is determined by one of ordinary skill in the art.

In certain embodiments, formulations provided herein comprise one or more glidants. Glidants may be used, e.g., to improve the flow properties of a powder composition or granulate or to improve the accuracy of dosing. Excipients that may function as glidants include, e.g., colloidal silicon dioxide, magnesium trisilicate, powdered cellulose, starch, tribasic calcium phosphate, calcium silicate, powdered cellulose, colloidal silicon dioxide, magnesium silicate, magnesium trisilicate, silicon dioxide, starch, tribasic calcium phosphate, and talc. In certain embodiments, the glidant is, relative to the drug core, present in the amount of less than about 1% w/w of the drug core, about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, about 10% w/w of the drug core, about 12% w/w of the drug core, about 14% w/w of the drug core, about 16% w/w of the drug core, about 18% w/w of the drug core, about 20% w/w of the drug core, about 22% w/w of the drug core, about 24% w/w of the drug core, about 26% w/w of the drug core, about 28% w/w of the drug core, about 30% w/w of the drug core, about 32% w/w of the drug core, between about 1% and about 10% w/w of the drug core, between about 2% and about 8% w/w of the drug core, between about 3% and about 7% w/w of the drug core, or between about 4% and about 6% w/w of the drug core. In certain embodiments, a suitable amount of a particular glidant is determined by one of ordinary skill in the art.

In certain embodiments, the pharmaceutical compositions provided herein may be administered orally, parenterally, by inhalation spray, topically, rectally, nasally, buccally, vaginally or via an implanted reservoir, preferably by oral administration or administration by injection. In one embodiment, the pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In certain embodiments, the pharmaceutical compositions provided herein may be in the form of a sterile injectable preparation, for example, as a sterile injectable aqueous or oleaginous suspension. This suspension may be formulated according to techniques known in the art using suitable dispersing or wetting agents (such as, for example, TWEEN 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs or SPANs and/or other similar emulsifying agents or bioavailability enhancers which are commonly used in the manufacture of pharmaceutically acceptable solid, liquid, or other dosage forms may also be used for the purposes of formulation.

In certain embodiments, the pharmaceutical compositions provided herein may also be administered in the form of suppositories for rectal administration. These compositions can be prepared by mixing a solid dispersion provided herein with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions provided herein is useful when the desired treatment involves areas or organs readily accessible by topical application. For application topically to the skin, the pharmaceutical composition should be formulated with a suitable ointment containing the active components suspended or dissolved in a carrier. In certain embodiments, carriers for topical administration of the compounds provided herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions provided herein may also be topically applied to the lower intestinal tract by rectal suppository formulation or in a suitable enema formulation. Topically-transdermal patches are also included herein.

In certain embodiments, the pharmaceutical compositions provided herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In certain embodiments, the compositions provided herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. In one embodiment, the pharmaceutical compositions are administered from about 1 to about 6 times per day or alternatively, as a continuous infusion. Such administration can be used as a chronic or acute therapy. The amount of active ingredient that may be combined with the carrier materials to produce a single dosage form varies depending upon the host treated and the particular mode of administration. A typical preparation contains from about 5% to about 95% active compound (w/w). Alternatively, such preparations contain from about 20% to about 80% active compound.

4. Methods of Use

The solid dispersions provided herein are useful for treating a disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, lessen the severity of the disease/disorder (i.e., a disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, each characterized by the presence of a mutant allele of IDH2. In one embodiment, the solid dispersion is amorphous.

In one embodiment, provided herein is a method of treating and preventing a disease or condition, comprising the administration of a solid dispersion comprising Compound 1, wherein the disease is selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, lessen the severity of the disease/disorder (i.e., a disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma, each characterized by the presence of a mutant allele of IDH2.

In one embodiment, provided herein is a method of treating AML selected from newly diagnosed AML, previously untreated AML, AML arising from MDS, AML arising from antecedent hematological disorder (AHD) and AML arising after exposure to genotoxic injury. In certain embodiments, the genotoxic injury is resulting from radiation and/or chemotherapy. In one embodiment, provided herein is a method of treating AML arising after exposure to genotoxic injury resulting from radiation and/or chemotherapy), each characterized by the presence of a mutant allele of IDH2.

In one embodiment, provided herein is a method of treating newly diagnosed AML characterized by the presence of a mutant allele of IDH2.

In one embodiment, provided herein is a method of treating previously untreated AML characterized by the presence of a mutant allele of IDH2.

In one embodiment, provided herein is a method of treating AML arising from MDS characterized by the presence of a mutant allele of IDH2.

In one embodiment, provided herein is a method of treating AML arising from AHD characterized by the presence of a mutant allele of IDH2.

In one embodiment, provided herein is a method of treating AML arising after exposure to genotoxic injury characterized by the presence of a mutant allele of IDH2.

In one embodiment, provided herein is a method of treating myeloproliferative neoplasm (MPN).

In one aspect of this embodiment, the mutant IDH2 has an R140X mutation. In another aspect of this embodiment, the R140X mutation is a R140Q mutation. In another aspect of this embodiment, the R140X mutation is a R140W mutation. In another aspect of this embodiment, the R140X mutation is a R140L mutation. In another aspect of this embodiment, the mutant IDH2 has an R172X mutation. In another aspect of this embodiment, the R172X mutation is a R172K mutation. In another aspect of this embodiment, the R172X mutation is a R172G mutation. A cancer selected from AML, MDS, CMML, or lymphoma (e.g., T-cell lymphoma) can be analyzed by sequencing cell samples to determine the presence and specific nature of (e.g., the changed amino acid present at) a mutation at amino acid 140 and/or 172 of IDH2.

Without being bound by theory, applicants believe that mutant alleles of IDH2 wherein the IDH2 mutation results in a new ability of the enzyme to catalyze the NADPH-dependent reduction of α-ketoglutarate to R(−)-2-hydroxyglutarate, and in particular R140Q and/or R172K mutations of IDH2, characterize a subset of all types of cancers described herein, without regard to their cellular nature or location in the body. Thus, the methods of one aspect are useful to treat a hematological cancer selected from AML, MDS, CMML, or lymphoma (e.g., T-cell lymphoma) or solid tumor selected from glioma, melanoma, chondrosarcoma, cholangiocarcinoma (e.g., glioma) and AITL, that is characterized by the presence of a mutant allele of IDH2 imparting such activity and in particular an IDH2 R140Q and/or R172K mutation.

In one embodiment, the efficacy of treatment is monitored by measuring the levels of 2HG in the subject. Typically levels of 2HG are measured prior to treatment, wherein an elevated level is indicated for the use of Compound 1 to treat the disease selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy. Similarly, a determination that 2HG levels are not elevated during the course of or following treatment is also indicative of efficacy. Typically, the these 2HG measurements will be utilized together with other well-known determinations of efficacy of cancer treatment, such as reduction in number and size of tumors and/or other cancer-associated lesions, improvement in the general health of the subject, and alterations in other biomarkers that are associated with cancer treatment efficacy.

2HG can be detected in a sample by the methods of PCT Publication No. WO 2013/102431 and US Publication No. US 2013/0190287 hereby incorporated by reference in their entirety, or by analogous methods.

In one embodiment 2HG is directly evaluated.

In another embodiment a derivative of 2HG formed in process of performing the analytic method is evaluated. By way of example such a derivative can be a derivative formed in MS analysis. Derivatives can include a salt adduct, e.g., a Na adduct, a hydration variant, or a hydration variant which is also a salt adduct, e.g., a Na adduct, e.g., as formed in MS analysis.

In another embodiment a metabolic derivative of 2HG is evaluated. Examples include species that build up or are elevated, or reduced, as a result of the presence of 2HG, such as glutarate or glutamate that will be correlated to 2HG, e.g., R-2HG.

Exemplary 2HG derivatives include dehydrated derivatives such as the compounds provided below or a salt adduct thereof:

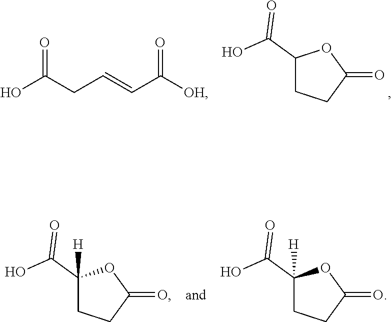 [SMILES: O=C(O)/C=C/CC(=O)O ; O=C1CCC(C(=O)O)O1 ; O=C1CC[C@H](C(=O)O)O1 ; "and" ; O=C1CC[C@@H](C(=O)O)O1]

In one embodiment the cancer selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma is a tumor wherein at least 30, 40, 50, 60, 70, 80 or 90% of the tumor cells carry an IDH2 mutation, and in particular an IDH2 R140Q, R140W, or R140L and/or R172K or R172G mutation, at the time of diagnosis or treatment.

In one embodiment, the cancer to be treated is AML. In some embodiments, the AML is relapsed and/or primary refractory. In some embodiments, the AML is relapsed and/or refractory. In other embodiments, the AML is previously untreated. In one embodiment, the AML is newly diagnosed AML.

In another embodiment, the cancer to be treated is MDS with refractory anemia with excess blasts (subtype RAEB-1 or RAEB-2). In other embodiments, the MDS is previously untreated. In one embodiment, the MDS is newly diagnosed MDS.

In another embodiment, the cancer to be treated is relapsed and/or primary refractory CMML.

In certain embodiments, the solid dispersions provided herein are for treating a hematological malignancy characterized by the presence of a mutant allele of IDH2 and the absence of a mutant allele of FLT3 and/or a mutant allele of NRAS. Exemplary methods for treating a hematological malignancy characterized by the presence of a mutant allele of IDH2 and the absence of a mutant allele of FLT3 and/or a mutant allele of NRAS by administering Compound 1 are described in US 2017/024617 and US 2017/0157132, the disclosure of each of which is incorporated herein by reference in its entirety.

In one embodiment, the solid dispersions provided herein are for treating a hematological malignancy characterized by the presence of a mutant allele of IDH2 and the absence of a mutant allele of FLT3. In one embodiment, the hematological malignancy is an advanced hematological malignancy. In one embodiment, the hematological malignancy is AML. In some embodiments, the AML is relapsed and/or refractory.

In one embodiment, provided herein are methods of treating a hematological malignancy by administering a solid dispersion comprising Compound 1 in combination with a therapeutically effective amount of one or more compounds that target a FLT3 pathway, wherein the hematological malignancy is characterized by the presence of a mutant allele of IDH2 and a mutant allele of FLT3, for example FLT3-ITD or FLT3-KDM. In one embodiment, the hematological malignancy is an advanced hematological malignancy. In one embodiment, the hematological malignancy is AML. In some embodiments, the AML is relapsed and/or refractory.

In one embodiment, provided herein is a method of treating hematological malignancies, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and the absence of a mutant allele of FLT3, comprising administering a solid dispersion comprising Compound 1. In one embodiment, the hematological malignancy is an advanced hematological malignancy. In one embodiment, the hematological malignancy is AML. In some embodiments, the AML is relapsed and/or refractory.

In one embodiment, provided herein is a method of treating hematological malignancies, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and a mutant allele of FLT3, for example FLT3-ITD, comprising administering a solid dispersion comprising Compound 1 in combination with a therapeutically effective amount of one or more compounds that target a FLT3 pathway. Exemplary FLT3 inhibitors are described elsewhere herein. In one embodiment, the hematological malignancy is an advanced hematological malignancy. In one embodiment, the hematological malignancy is AML. In some embodiments, the AML, is relapsed and/or refractory.

In one embodiment, provided herein are methods of treating solid tumors by administering a solid dispersion comprising Compound 1, wherein the solid tumor is characterized by the presence of a mutant allele of IDH2 and the absence of a mutant allele of FLT3. In one embodiment, the solid tumor is an advanced solid tumor. In some embodiments, the AML is relapsed and/or refractory.

In one embodiment, provided herein are methods of treating solid tumors by administering to a subject a solid dispersion comprising Compound 1 in combination with a therapeutically effective amount of one or more compounds that target a FLT3 pathway, wherein the solid tumor is characterized by the presence of a mutant IDH2 and a mutant allele of FLT3, for example FLT3-ITD. In one embodiment, the solid tumor is an advanced solid tumor.

In one embodiment, provided herein is a method of treating solid tumors, such as glioma, melanoma, chondrosarcoma, or cholangiocarcinoma(e.g., glioma), or treating AITL, each characterized by the presence of a mutant allele of IDH2 and the absence of a mutant allele of FLT3, comprising administering to a subject a solid dispersion provided herein.

In one embodiment, provided herein is a method of treating solid tumors, such as glioma, melanoma, chondrosarcoma, or cholangiocarcinoma (e.g., glioma), or treating AITL, each characterized by the presence of a mutant allele of IDH2 and a mutant allele of FLT3, in a subject comprising administering a solid dispersion comprising Compound 1 in combination with a therapeutically effective amount of one or more compounds that target a FLT3 pathway. Exemplary FLT3 inhibitors are described elsewhere herein.

In one embodiment, provided herein is a method of treating a hematological malignancy by administering a solid dispersion comprising Compound 1, wherein the hematological malignancy is characterized by the presence of a mutant allele of IDH2 and the absence of a mutant allele of NRAS. In one embodiment, the hematological malignancy is an advanced hematological malignancy.

In one embodiment, provided herein is a method of treating a hematological malignancy by administering a solid dispersion comprising Compound 1 in combination with a therapeutically effective amount of one or more compounds that target RAS pathways, wherein the hematological malignancy is characterized by the presence of a mutant allele of IDH2 and a mutant allele of NRAS. In one embodiment, the hematological malignancy is an advanced hematological malignancy.

In one embodiment, provided herein is a method of treating a hematological malignancy, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and the absence of a mutant allele of NRAS, comprising administering a solid dispersion comprising Compound 1. In one embodiment, the hematological malignancy is an advanced hematological malignancy.

In one embodiment, provided herein is a method of treating hematological malignancies, such as AML, MDS, CMML, myeloid sarcoma, multiple myeloma, lymphoma (e.g., T-cell lymphoma or B-cell lymphoma), AITL or blastic plasmacytoid dendritic cell neoplasm, each characterized by the presence of a mutant allele of IDH2 and a mutant allele of NRAS comprising administering a solid dispersion comprising Compound 1 in combination with a therapeutically effective amount of one or more compounds that target RAS pathways. In one embodiment, a solid dispersion comprising Compound 1 is administered to the subject in combination with a therapeutically effective amount of a MEK kinase inhibitor. Exemplary MEK kinase inhibitors are described elsewhere herein. In one embodiment, the hematological malignancy is an advanced hematological malignancy.

In one embodiment, provided herein are methods of treating solid tumors by administering a solid dispersion comprising Compound 1, wherein the solid tumor is characterized by the presence of a mutant allele of IDH2 and the absence of a mutant allele of NRAS. In one embodiment, the solid tumor is an advanced solid tumor.

In one embodiment, provided herein are methods of treating solid tumors by administering a solid dispersion comprising Compound 1 in combination with a therapeutically effective amount of one or more compounds that target RAS pathways, wherein the solid tumor is characterized by the presence of a mutant IDH2 and a mutant allele of NRAS. In one embodiment, the solid tumor is an advanced solid tumor.

In one embodiment, provided herein is a method of treating solid tumors, such as glioma, melanoma, chondrosarcoma, or cholangiocarcinoma(e.g., glioma), or treating angioimmunoblastic T-cell lymphoma (AITL), each characterized by the presence of a mutant allele of IDH2 and the absence of a mutant allele of NRAS, comprising administering a solid dispersion comprising Compound 1.

In one embodiment, provided herein is a method of treating solid tumors, such as glioma, melanoma, chondrosarcoma, or cholangiocarcinoma (e.g., glioma), or treating angioimmunoblastic T-cell lymphoma (AITL), each characterized by the presence of a mutant allele of IDH2 and a mutant allele of NRAS, comprising administering a solid dispersion comprising Compound 1 in combination with a therapeutically effective amount of one or more compounds that target RAS pathways.

In one embodiment, provided herein are methods of treating MPN in a subject comprising administering to the subject a solid dispersion comprising Compound 1 in combination with a therapeutically effective amount of a JAK2 inhibitor, wherein the subject harbors a mutant allele of IDH2 and a mutant allele of JAK2. Exemplary JAK2 inhibitors are described elsewhere herein.

In certain embodiments, provided herein is a method of treating a high risk MPN in a subject comprising administering to the subject a solid dispersion comprising Compound 1 in combination with a therapeutically effective amount of a JAK2 inhibitor, wherein the subject harbors a mutant allele of IDH2 and a mutant allele of JAK2.

In one embodiment, provided herein are methods of treating AML in a subject comprising administering to the subject a solid dispersion comprising Compound 1 in combination with a therapeutically effective amount of a JAK2 inhibitor, wherein the subject harbors a mutant allele of IDH2 and a mutant allele of JAK2. In some embodiments, the AML is relapsed and/or refractory.

In certain embodiments, the mutant allele of IDH2 is mIDH2-R140 or mIDH2-R172.

In certain embodiments, the mutant allele of IDH2 is mIDH2-R140Q, mIDH2-R140W, mIDH2-R140L, mIDH2-R172K, or mIDH2-R172G.

In certain embodiments, the mutant allele of JAK2 is mJAK2-V617F.

In certain embodiments, the solid dispersions provided herein are for treating MDS characterized by the presence of a mutant allele of IDH2 and a mutant allele of at least one second gene, wherein the second gene is selected from the group consisting of ASXL1 and SRSF2. In certain embodiments, the solid dispersions provided herein are for treating MDS characterized by the presence of a mutant allele of IDH2 and the absence of a mutant allele of at least one other gene, wherein the other gene is selected from the group consisting of KRAS, TP53, SETBP1, and U2AF1. In certain embodiments, the solid dispersions provided herein are for treating MDS characterized by the presence of a mutant allele of IDH2 and the absence of a mutant allele of at least one other gene, wherein the other gene is selected from the group consisting of KRAS, TP53, SETBP1, U2AF1, TCF3, STAG2, NRAS, JAK2 and BRAF. Exemplary methods of treating MDS characterized by the presence of a mutant allele of IDH2 by administering Compound 1 are described in US 2018/0042930-A1, the disclosure of which is incorporated herein by reference in its entirety.

In one embodiment, prior to and/or after treatment with a solid dispersion comprising Compound 1 provided herein, the method further comprises the step of evaluating the growth, size, weight, invasiveness, stage and/or other phenotype of the cancer selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma.

In one embodiment, prior to and/or after treatment with a composition provided herein, the method further comprises the step of evaluating the IDH2 genotype of the cancer selected from AML, MDS, CMML, myeloid sarcoma, multiple myeloma, lymphoma (e.g., T-cell lymphoma or B-cell lymphoma), AITL, blastic plasmacytoid dendritic cell neoplasm, MPN, glioma, melanoma, chondrosarcoma, and cholangiocarcinoma. This may be achieved by ordinary methods in the art, such as DNA sequencing, immuno analysis, and/or evaluation of the presence, distribution or level of 2HG.

In one embodiment, prior to and/or after treatment with a composition provided herein, the method further comprises the step of determining the 2HG level in the subject. This may be achieved by spectroscopic analysis, e.g., magnetic resonance-based analysis, e.g., MRI and/or MRS measurement, sample analysis of bodily fluid, such as blood, plasma, urine, or spinal cord fluid analysis, or by analysis of surgical material, e.g., by mass-spectroscopy (e.g. LC-MS, GC-MS).

In one embodiment, the solid dispersion comprising Compound 1 is for use in any of the above described methods. In one embodiment, the solid dispersion is amorphous.

In certain embodiments, depending on the disease to be treated and the subject's condition, the solid dispersion provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The solid dispersion provided herein may be formulated alone or together with one or more active agent(s), in suitable dosage unit with pharmaceutically acceptable excipients, carriers, adjuvants and vehicles, appropriate for each route of administration.

In certain embodiments, the amount of the solid dispersion provided herein administered in the methods provided herein may range, e.g., between about 5 mg/day and about 2,000 mg/day. In one embodiment, the range is between about 10 mg/day and about 2,000 mg/day. In one embodiment, the range is between about 20 mg/day and about 2,000 mg/day. In one embodiment, the range is between about 50 mg/day and about 1,000 mg/day. In one embodiment, the range is between about 100 mg/day and about 1,000 mg/day. In one embodiment, the range is between about 100 mg/day and about 500 mg/day. In one embodiment, the range is between about 150 mg/day and about 500 mg/day. In one embodiment, the range is or between about 150 mg/day and about 250 mg/day. In certain embodiments, particular dosages are, e.g., about 10 mg/day. In one embodiment, the dose is about 20 mg/day. In one embodiment, the dose is about 50 mg/day. In one embodiment, the dose is about 60 mg/day. In one embodiment, the dose is about 75 mg/day. In one embodiment, the dose is about 100 mg/day. In one embodiment, the dose is about 120 mg/day. In one embodiment, the dose is about 150 mg/day. In one embodiment, the dose is about 200 mg/day. In one embodiment, the dose is about 250 mg/day. In one embodiment, the dose is about 300 mg/day. In one embodiment, the dose is about 350 mg/day. In one embodiment, the dose is about 400 mg/day. In one embodiment, the dose is about 450 mg/day. In one embodiment, the dose is about 500 mg/day. In one embodiment, the dose is about 600 mg/day. In one embodiment, the dose is about 700 mg/day. In one embodiment, the dose is about 800 mg/day. In one embodiment, the dose is about 900 mg/day. In one embodiment, the dose is about 1,000 mg/day. In one embodiment, the dose is about 1,200 mg/day. In one embodiment, the dose is or about 1,500 mg/day. In certain embodiments, particular dosages are, e.g., up to about 10 mg/day. In one embodiment, the particular dose is up to about 20 mg/day. In one embodiment, the particular dose is up to about 50 mg/day. In one embodiment, the particular dose is up to about 60 mg/day. In one embodiment, the particular dose is up to about 75 mg/day. In one embodiment, the particular dose is up to about 100 mg/day. In one embodiment, the particular dose is up to about 120 mg/day. In one embodiment, the particular dose is up to about 150 mg/day. In one embodiment, the particular dose is up to about 200 mg/day. In one embodiment, the particular dose is up to about 250 mg/day. In one embodiment, the particular dose is up to about 300 mg/day. In one embodiment, the particular dose is up to about 350 mg/day. In one embodiment, the particular dose is up to about 400 mg/day. In one embodiment, the particular dose is up to about 450 mg/day. In one embodiment, the particular dose is up to about 500 mg/day. In one embodiment, the particular dose is up to about 600 mg/day. In one embodiment, the particular dose is up to about 700 mg/day. In one embodiment, the particular dose is up to about 800 mg/day. In one embodiment, the particular dose is up to about 900 mg/day. In one embodiment, the particular dose is up to about 1,000 mg/day. In one embodiment, the particular dose is up to about 1,200 mg/day. In one embodiment, the particular dose is up to about 1,500 mg/day.

In certain embodiments, the solid dispersion provided herein for the methods described herein is administered at a dose of about 20 to 2000 mg/day. In certain embodiments, the solid dispersion provided herein is administered at a dose of about 50 to 500 mg/day. In certain embodiments, the dose is about 60 mg/day. In certain embodiments, the dose is about 100 mg/day. In certain embodiments, the dose is about 150 mg/day. In certain embodiments, the dose is about 200 mg/day. In certain embodiments, the dose is about 300 mg/day.

In one embodiment, the amount of the solid dispersion provided herein in the pharmaceutical composition or dosage form provided herein may range, e.g., between about 5 mg and about 2,000 mg. In one embodiment, the range is between about 10 mg and about 2,000 mg. In one embodiment, the range is between about 20 mg and about 2,000 mg. In one embodiment, the range is between about 50 mg and about 1,000 mg. In one embodiment, the range is between about 50 mg and about 500 mg. In one embodiment, the range is between about 50 mg and about 250 mg. In one embodiment, the range is between about 100 mg and about 500 mg. In one embodiment, the range is between about 150 mg and about 500 mg. In one embodiment, the range is between about 150 mg and about 250 mg. In certain embodiments, particular amounts are, e.g., about 10 mg. In one embodiment, the particular amount is about 20 mg. In one embodiment, the particular amount is about 30 mg. In one embodiment, the particular amount is about 50 mg. In one embodiment, the particular amount is about 60 mg. In one embodiment, the particular amount is about 75 mg. In one embodiment, the particular amount is about 100 mg. In one embodiment, the particular amount is about 120 mg. In one embodiment, the particular amount is about 150 mg. In one embodiment, the particular amount is about 200 mg. In one embodiment, the particular amount is about 250 mg. In one embodiment, the particular amount is about 300 mg. In one embodiment, the particular amount is about 350 mg. In one embodiment, the particular amount is about 400 mg. In one embodiment, the particular amount is about 450 mg. In one embodiment, the particular amount is about 500 mg. In one embodiment, the particular amount is about 600 mg. In one embodiment, the particular amount is about 650 mg. In one embodiment, the particular amount is about 700 mg. In one embodiment, the particular amount is about 800 mg. In one embodiment, the particular amount is about 900 mg. In one embodiment, the particular amount is about 1,000 mg. In one embodiment, the particular amount is about 1,200 mg. In one embodiment, the particular amount is or about 1,500 mg. In certain embodiments, particular amounts are, e.g., up to about 10 mg. In one embodiment, the particular amount is up to about 20 mg. In one embodiment, the particular amount is up to about 50 mg. In one embodiment, the particular amount is up to about 60 mg. In one embodiment, the particular amount is up to about 75 mg. In one embodiment, the particular amount is up to about 100 mg. In one embodiment, the particular amount is up to about 120 mg. In one embodiment, the particular amount is up to about 150 mg. In one embodiment, the particular amount is up to about 200 mg. In one embodiment, the particular amount is up to about 250 mg. In one embodiment, the particular amount is up to about 300 mg. In one embodiment, the particular amount is up to about 350 mg. In one embodiment, the particular amount is up to about 400 mg. In one embodiment, the particular amount is up to about 450 mg. In one embodiment, the particular amount is up to about 500 mg. In one embodiment, the particular amount is up to about 600 mg. In one embodiment, the particular amount is up to about 700 mg. In one embodiment, the particular amount is up to about 800 mg. In one embodiment, the particular amount is up to about 900 mg. In one embodiment, the particular amount is up to about 1,000 mg. In one embodiment, the particular amount is up to about 1,200 mg. In one embodiment, the particular amount is up to about 1,500 mg.

In one embodiment, the solid dispersion provided herein can be delivered as a single dose such as, e.g., a single bolus injection, or oral tablets or pills; or over time such as, e.g., continuous infusion over time or divided bolus doses over time. In one embodiment, the solid dispersion provided herein can be administered repetitively if necessary, for example, until the patient experiences stable disease or regression, or until the patient experiences disease progression or unacceptable toxicity. Stable disease or lack thereof is determined by methods known in the art such as evaluation of patient's symptoms, physical examination, visualization of the tumor that has been imaged using X-ray, CAT, PET, or MM scan and other commonly accepted evaluation modalities.

In certain embodiments, the solid dispersion provided herein for methods described herein is administered once daily.

In certain embodiments, the solid dispersion provided herein is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, a method provided herein comprises administering the solid dispersion provided herein in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In certain embodiments, the solid dispersion provided herein for methods described herein is administered for 1 to 25 cycles. In one embodiment, the median number of cycles administered in a group of patients is about 1. In one embodiment, the median number of cycles administered in a group of patients is about 2. In one embodiment, the median number of cycles administered in a group of patients is about 3. In one embodiment, the median number of cycles administered in a group of patients is about 4. In one embodiment, the median number of cycles administered in a group of patients is about 5. In one embodiment, the median number of cycles administered in a group of patients is about 6. In one embodiment, the median number of cycles administered in a group of patients is about 7. In one embodiment, the median number of cycles administered in a group of patients is about 8. In one embodiment, the median number of cycles administered in a group of patients is about 9. In one embodiment, the median number of cycles administered in a group of patients is about 10. In one embodiment, the median number of cycles administered in a group of patients is about 11. In one embodiment, the median number of cycles administered in a group of patients is about 12. In one embodiment, the median number of cycles administered in a group of patients is about 13. In one embodiment, the median number of cycles administered in a group of patients is about 14. In one embodiment, the median number of cycles administered in a group of patients is about 15. In one embodiment, the median number of cycles administered in a group of patients is about 16. In one embodiment, the median number of cycles administered in a group of patients is about 17. In one embodiment, the median number of cycles administered in a group of patients is about 18. In one embodiment, the median number of cycles administered in a group of patients is about 19. In one embodiment, the median number of cycles administered in a group of patients is about 20. In one embodiment, the median number of cycles administered in a group of patients is about 21. In one embodiment, the median number of cycles administered in a group of patients is about 22. In one embodiment, the median number of cycles administered in a group of patients is about 23. In one embodiment, the median number of cycles administered in a group of patients is about 24. In one embodiment, the median number of cycles administered in a group of patients is about 25. In one embodiment, the median number of cycles administered in a group of patients is about 26. In one embodiment, the median number of cycles administered in a group of patients is about 27. In one embodiment, the median number of cycles administered in a group of patients is about 28. In one embodiment, the median number of cycles administered in a group of patients is about 29. In one embodiment, the median number of cycles administered in a group of patients is about 30. In one embodiment, the median number of cycles administered in a group of patients is greater than about 30 cycles.

In certain embodiments, treatment cycles comprise multiple doses of the solid dispersion provided herein administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days).

In certain embodiments, the solid dispersion provided herein is administered in one or more 28 day cycles in the methods described herein. In certain embodiments, the solid dispersion provided herein is administered in a 28 day cycle in the methods described herein.

In certain embodiments, the solid dispersion provided herein is administered orally in the methods described herein.

In certain embodiments, the solid dispersion provided herein is administered once daily orally in 28-day cycles at the dose of about 100 mg/day in the methods described herein.

5. Combination Therapy

In certain embodiments, the solid dispersions provided herein are used with an additional cancer therapeutic agent or an additional cancer treatment. Exemplary additional cancer therapeutic agents and additional cancer treatments are described in US 2013/0190287, US 2017/0157132, US 2017/0246174, WO 2017/066611, WO 2017/066599 and International Application No. PCT/US18/31090, the disclosures of each of which is incorporated herein by reference in their entireties.

In certain embodiments, additional cancer therapeutic agents include for example, chemotherapy, targeted therapy, antibody therapies, immunotherapy, and hormonal therapy. In certain embodiments, additional cancer treatments include, for example: surgery, and radiation therapy. Examples of each of these treatments are provided below.

In some embodiments, the additional cancer therapeutic agent is a chemotherapy agent. Examples of chemotherapeutic agents used in cancer therapy include, for example, antimetabolites (e.g., folic acid, purine, and pyrimidine derivatives), alkylating agents (e.g., nitrogen mustards, nitrosoureas, platinum, alkyl sulfonates, hydrazines, triazenes, aziridines, spindle poison, cytotoxic agents, topoisomerase inhibitors and others), and hypomethylating agents (e.g., decitabine (5-aza-deoxycytidine), zebularine, isothiocyanates, azacitidine (5-azacytidine), 5-flouro-2′-deoxycytidine, 5,6-dihydro-5-azacytidine and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time. Often, two or more chemotherapy agents are used as combination chemotherapy.

In some embodiments, the additional cancer therapeutic agent is a differentiation agent. Such differentiation agent includes retinoids (such as all-trans-retinoic acid (ATRA), 9-cis retinoic acid, 13-cis-retinoic acid (13-cRA) and 4-hydroxy-phenretinamide (4-HPR)); arsenic trioxide; histone deacetylase inhibitors HDACs (such as azacytidine (Vidaza) and butyrates (e.g., sodium phenylbutyrate)); hybrid polar compounds (such as hexamethylene bisacetamide ((HMBA)); vitamin D; and cytokines (such as colony-stimulating factors including G-CSF and GM-CSF, and interferons).

In some embodiments the additional cancer therapeutic agent is a targeted therapy agent. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin dependent kinase inhibitors such as Alvocidib and Seliciclib. Monoclonal antibody therapy is another strategy in which the therapeutic agent is an antibody which specifically binds to a protein on the surface of the cancer cells. Examples include the anti HER2/neu antibody trastuzumab (HERCEPTIN®) typically used in breast cancer, and the anti CD20 antibody rituximab and Tositumomab typically used in a variety of B cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox. In some embodiments, the targeted therapy can be used in combination with a compound described herein, e.g., a biguanide such as metformin or phenformin, preferably phenformin.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

In some embodiments, the additional cancer therapeutic agent is an immunotherapy agent. Cancer immunotherapy refers to a diverse set of therapeutic strategies designed to induce the subject's own immune system to fight the tumor. Contemporary methods for generating an immune response against tumors include intravesicular BCG immunotherapy for superficial bladder cancer, and use of interferons and other cytokines to induce an immune response in renal cell carcinoma and melanoma subjects.

Allogeneic hematopoietic stem cell transplantation can be considered a form of immunotherapy, since the donor's immune cells will often attack the tumor in a graft versus tumor effect. In some embodiments, the immunotherapy agents can be used in combination with a compound or composition described herein.

In some embodiments, the additional cancer therapeutic agent is a hormonal therapy agent. The growth of some cancers can be inhibited by providing or blocking certain hormones. Common examples of hormone sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with a compound or a composition described herein.

Other possible additional therapeutic modalities include imatinib, gene therapy, peptide and dendritic cell vaccines, synthetic chlorotoxins, and radiolabeled drugs and antibodies.

In one embodiment, the compositions provided herein are used for treatment of AML in combination with an AML induction and consolidation therapy. In one embodiment, the AML induction therapy is a combination of cytarabine and daunorubicin. In one embodiment, the AML induction therapy is a combination of cytarabine and idarubicin.

In one embodiment, the AML consolidation therapy is cytarabine. In one embodiment, the AML consolidation therapy is a combination of mitoxantrone and etoposide.

In one embodiment, the compositions provided herein are used in combination with one or more DNA demethylating agents. In one embodiment, the DNA demethylating agent is a cytidine analog. In certain embodiments, the cytidine analog is azacitidine or 5-aza-2′-deoxycytidine (decitabine). In certain embodiments, the cytidine analog is azacitidine. In certain embodiments, the cytidine analog is 5-aza-2′-deoxycytidine (decitabine). In certain embodiments, the cytidine analog is, for example: 1-β-D-arabinofuranosylcytosine (cytarabine or ara-C); pseudoiso-cytidine (psi ICR); 5-fluoro-2′-deoxycytidine (FCdR); 2′-deoxy-2′,2′-difluorocytidine (gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (zebularine); 2′,3′-dideoxy-5-fluoro-3′-thiacytidine (emtriva); 2′-cyclocytidine (ancitabine); 1-β-D-arabinofuranosyl-5-azacytosine (fazarabine or ara-AC); 6-azacitidine (6-aza-CR); 5,6-dihydro-5-azacitidine (dH-aza-CR); N⁴ pentyloxy-carbonyl-5′-deoxy-5-fluorocytidine (capecitabine); N⁴ octadecyl-cytarabine; or elaidic acid cytarabine. In certain embodiments, the cytidine analogs include any compound which is structurally related to cytidine or deoxycytidine and functionally mimics and/or antagonizes the action of cytidine or deoxycytidine.

In one embodiment, the compositions provided herein are used in combination with azacitidine.

In one embodiment, the compositions provided herein are used in combination with a FLT3 inhibitor. In one embodiment, the FLT3 inhibitor is selected from quizartinib (AC220), sunitinib (SU11248), sorafenib (BAY 43-9006), midostaurin (PKC412), crenolanib (CP-868596), PLX3397, E6201, AKN-028, ponatinib (AP24534), ASP2215, KW-2449, famitinib and DCC-2036.

In one embodiment, the compositions provided herein are used in combination with MEK kinase inhibitor. In one embodiment, the MEK kinase is selected from trametinib, selumetinib, binimetinib, PD-325901, cobimetinib, CI-1040 and PD035901.

In one embodiment, the compositions provided herein are used in combination with a JAK inhibitor. In one embodiment, the compositions provided herein are used in combination with a JAK2 inhibitor. In one embodiment, the JAK2 inhibitor is selected from INCB018424 (ruxolitinib), TG101348, CYT387, AZD1480, SB1518 (pacritinib), XL019, NCB0-16562, NVP-BSK805, R723, hydroxycarbamide, SAR302503, CP-690,550 (tasocitinib) and INCB16562. In one embodiment, the compositions provided herein are used in combination with ruxolitinib.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the methods of use provided herein, may be made without departing from the spirit and scope thereof. Patents, patent publications, and other publications referenced herein are incorporated by reference.

### EXAMPLES

The embodiments described below are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

The following abbreviations are used:

### Abbreviation of Chemicals

DCM Dichloromethane

DMSO Dimethyl sulfoxide

EtOH Ethanol

HEC Hydroxyethyl cellulose

HPC Hydroxypropyl cellulose

HPMC Hydroxypropyl methylcellulose

MeOH Methanol

PEG Polyethylene glycol

PEO Polyethylene oxide

PVP Polyvinylpyrrolidone

PVP-VA Poly(1-vinylpyrrolidone-co-vinyl acetate)

SGF Simulated Gastric Fluid test solution

SIF Simulated Intestinal Fluid test solution

THF Tetrahydrofuran

TFE Trifluoroethanol

TPGS D-α-Tocopherol Polyethylene Glycol Succinate

### Other Abbreviations

AAC Accelerated Aging Conditions (4 weeks at 40° C. and 70% RH)

Am Amorphous

API Active Pharmaceutical Ingredient

ASD Experiment ID for the amorphous solid dispersion experiments

DSC Differential Scanning calorimetry

HPLC High-Performance Liquid Chromatography

HR-XRPD High Resolution X-Ray Powder Diffraction

HT-XRPD High Throughput X-Ray Powder Diffraction

MS Mass Spectroscopy

QSA Experiment ID of the solubility experiments

REF Experiment ID for the anti-precipitant experiments

RH Relative Humidity

RT Room Temperature

SDTA Single Differential Thermal Analysis

SM Starting Material

TGA Thermo Gravimetric Analysis

TGMS Thermo Gravimetric Analysis coupled with Mass Spectroscopy

Analytical Methods

High Throughput X-Ray Powder Diffraction

HT-XRPD patterns were obtained using the Crystallics T2 high-throughput XRPD set-up. The plates were mounted on a Bruker General Area Detector Diffraction System (GADDS) equipped with a VÅNTEC-500 gas area detector corrected for intensity and geometric variations. The calibration of the measurement accuracy (peaks position) was performed using NIST SRM1976 standard (Corundum).

Data collection was carried out at room temperature using monochromatic CuKα radiation in the 2 Å region between 1.5° and 41.5°, which is the most distinctive part of the XRPD pattern. The diffraction pattern of each well was collected in two 20 ranges (1.5°≤2θ≤21.5° for the first frame, and 19.5°≤2θ≤41.5° for the second) with an exposure time of 90s for each frame. No background subtraction or curve smoothing was applied to the XRPD patterns.

The carrier material used during XRPD analysis was transparent to X-rays and contributed only slightly to the background.

Thermal Analysis

DSC Analysis

Melting properties were obtained from DSC thermograms, recorded with a heat flux DSC822e instrument (Mettler-Toledo GmbH, Switzerland). The DSC822e was calibrated for temperature and enthalpy with a small piece of indium (melting point at 156.6° C.; ΔHf=28.45 J/g). Samples were sealed in standard 40 μL aluminum pans, pin-holed and heated in the DSC from 25° C. to 300° C., at a heating rate of 10° C./min. Dry N2 gas, at a flow rate of 50 mL/min was used to purge the DSC equipment during the measurement.

TGMS Analysis

Mass loss due to solvent or water loss from the crystals was determined by TGA/SDTA. Monitoring the sample weight, during heating in a TGA/SDTA851e instrument (Mettler-Toledo GmbH, Switzerland), which resulted in a weight vs. temperature curve. The TGA/SDTA851e was calibrated with samples of indium and aluminum. Samples were weighed into 100 μL aluminum crucibles and sealed. The seals were pin-holed and the crucibles heated in the TGA from 25 to 300° C. at a heating rate of 10° C./min. Dry N2 gas was used for purging.

The gases coming from the TGA samples were analyzed by a mass spectrometer Omnistar GSD 301 T2 (Pfeiffer Vacuum GmbH, Germany). The latter is a quadrupole mass spectrometer, which analyzes masses in the range of 0-200 amu.

HPLC Analytical Method

Method name: S16124_01

HPLC System:

HPLC: Agilent 1200

Detector 1: DAD set at 270 nm

Detector 2: HP1100 LC/MSD in Positive Scan mode

HPLC Conditions:

Auto sampler temp: 20° C.

Column: Waters Sunfire C18 (100×4.6 mm; 3.5 μm).

Column temp: 35° C.

Flow cell: 10 mm path

Gradient: Mobile phase A: 10 mM Ammonium acetate

Mobile phase B: Acetonitrile

Flow: 1.0 ml/min

Gradient:

| Time [min]: | Eluent A: | Eluent B: |
|---|---|---|
| 0 | 90% | 10% |
| 1 | 90% | 10% |
| 6 | 10% | 90% |
| 9 | 10% | 90% |
| 10 | 90% | 10% |

Sample:

Concentration: ca. 0.2 mg/ml

Solvent: acetonitrile

Injection volume: 5 μl

Retention time API: 6.7 min

The compound integrity is expressed as a peak-area percentage, calculated from the area of each peak in the chromatogram, except the ‘injection peak’, and the total peak-area, as follows:

$$peak area ( \% ) = \frac{peak area}{total area of all peaks} \cdot 100 \%$$

The peak area percentage of the compound of interest is employed as an indication of the purity of the component in the sample.

The peak area of the API was used to calculate the concentration of solute for the solvent shift method and solubility determination experiments.

### Example 1 Compound 1A—Starting Material Characterization

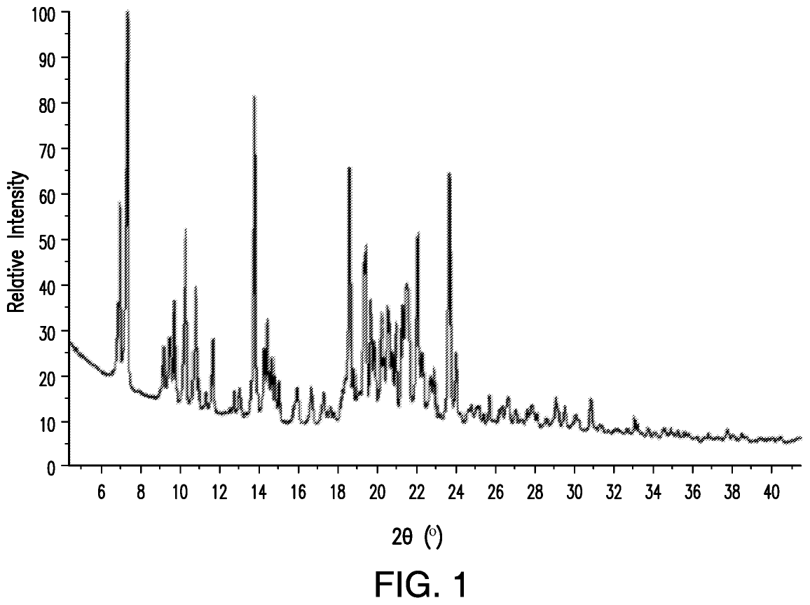

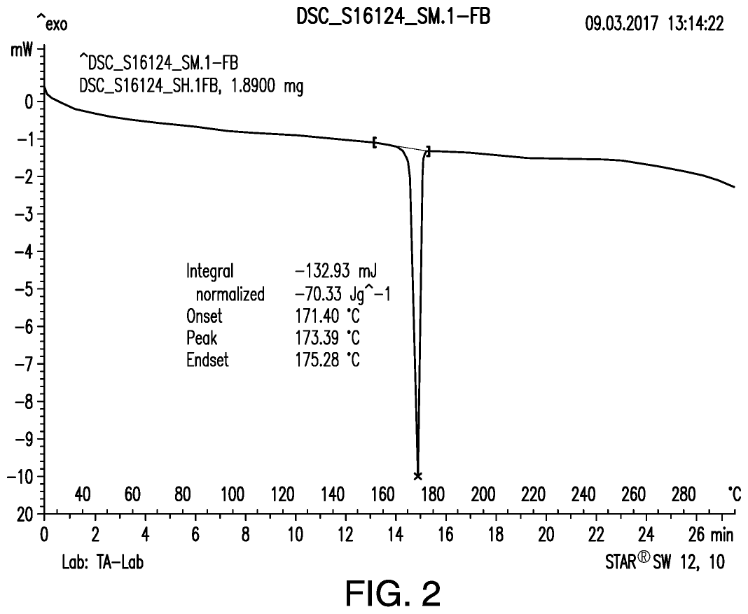

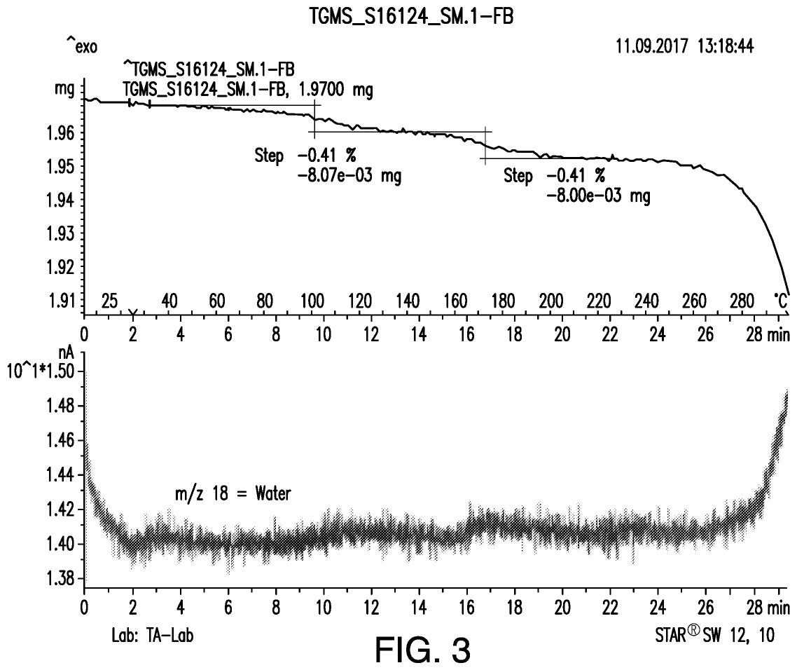

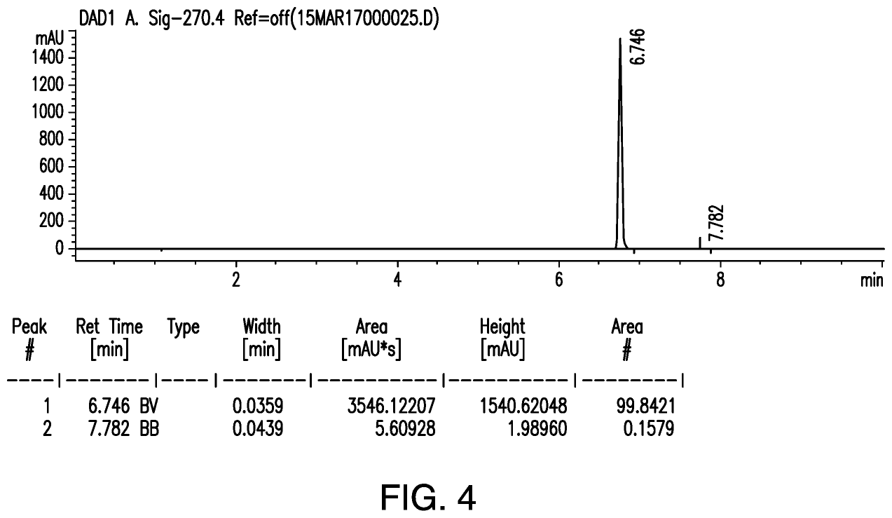

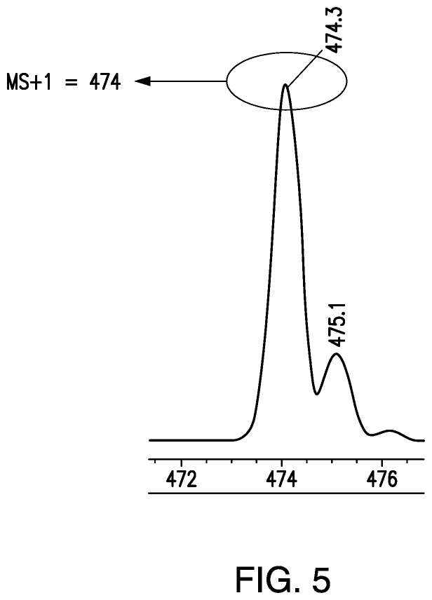

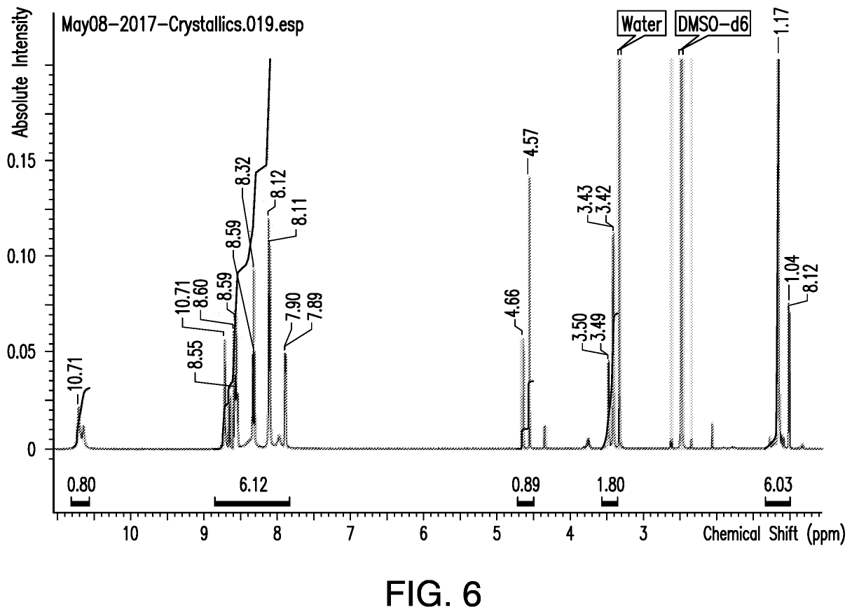

Compound 1A used in the following examples was a crystalline solid which was a mixture of two crystalline phases Form 17 and Form A. The High Resolution XRPD analysis on the starting material is provided in FIG. 1. The material was also characterized by thermal analysis, LCMS and proton NMR. The DSC thermogram indicated a melting point of 173.4° C. (FIG. 2). A mass loss of 0.4% was observed prior to melting, possibly related to residual process solvents. The thermal decomposition occurred above 240° C. (FIG. 3). The HPLC profile and MS data of the starting material are provided in FIGS. 4 and 5, respectively. The main peak in HPLC was observed at a retention time of 6.7 min with a chemical purity of 99.8% (area %). The MS signal confirmed the molecular weight of 473 g/mol corresponding to the molecular weight of Compound 1A. The ¹H-NMR is of the starting material is provided in FIG. 6.

The list of polymers used in this study is provided in Table 1 below:

TABLE 1

| Description | Supplier |
|---|---|
| HPMC-AS-MG | Ashland Industries Europe GmbH |
| HPMC-AS-LG | Ashland Industries Europe GmbH |
| HPMC-AS-HG | Ashland Industries Europe GmbH |
| HPMC E5 | Aldrich |
| HPMC-P-55S | Shin-Etsu Chemical Co. |
| HPMC-P-50 | Acros |
| Methyl Cellulose | Sigma-Aldrich |
| HEC | Sigma-Aldrich |
| HPC | Sigma-Aldrich |
| Eudragit L100 | Evonik - Degussa |
| Eudragit E100 | Evonik - Degussa |
| PEO 100K | Sigma-Aldrich |
| PEG 6000 | Fluka |
| PVP VA64 | BASF |
| PVP K30 | Fluka |
| TPGS | Sigma-Aldrich |
| Kollicoat IR | Sigma-Aldrich |
| Carbopol ® 980NF | Lubrizol |
| Povacoat Type MP | DAIDO-Chem |
| Soluplus ® | BASF |
| Sureteric | Colorcon |
| Pluronic F-68 | Sigma-Aldrich |

All other chemicals were obtained from Fisher Scientific, Sigma Aldrich or VWR. Chemicals used were of research grade and the chemical used for HPLC analysis were of HPLC grade.

### Example 2: Solubility of Compound 1A

A quantitative (24-hour shake flask) thermodynamic solubility determination was performed on Compound 1A to aid in the selection of the screening solvents.

The solubility of Compound 1A was quantitatively assessed in 10 different organic solvents and water. Slurries were prepared by mixing 30 mg of Compound 1A with an appropriate solvent volume. A stirring bean was added and the suspensions were stirring (500 rpm) at room temperature for 24 hours. An aliquot of the suspension was transferred into a spin filter (nylon, 0.2 μm). The solubility of the filtered solution was determined by HPLC. The experimental conditions are presented in Table 2.

TABLE 2
Experimental conditions and results for the quantitative
solubility determination performed on Compound 1A..

| | | Mass | Solvent | Slurry | | |
|---|---|---|---|---|---|---|
| | | API | Volume | concentration | | Solubility |
| Exp ID | Solvent | (mg) | (μL) | (mg/mL) | Slurry? | (mg/mL) |
| QSA1 | Tetrahydrofuran | 49.1 | 100 | 491 | No | >429 |
| QSA2 | Acetone | 56.0 | 200 | 280 | Yes | 209 |
| QSA3 | Acetonitrile | 50.3 | 300 | 168 | Yes | 14.7 |
| QSA4 | Water | 37.1 | 1000 | 37 | Yes | <0.1 |
| QSA5 | Methanol | 53.3 | 100 | 533 | No | >463 |
| QSA6 | Ethanol | 48.6 | 100 | 488 | No | >408 |
| QSA7 | 2-Propanol | 46.6 | 200 | 233 | Yes | 229 |
| QSA8 | Ethyl acetate | 28.9 | 200 | 145 | Yes | 127 |
| QSA9 | 1,4-Dioxane | 48.7 | 300 | 162 | Yes | 69.2 |
| QSA10 | Chloroform | 47.9 | 250 | 192 | Yes | 70.6 |

The residual solids were analyzed by HT-XRPD. In several solvents, conversion to a single crystalline phase was observed (i.e. in 2-propanol and chloroform conversion to Form 17 was observed while in acetone and ethyl formate conversion to Form A was observed). In 1,4-dioxane and water, conversion to Form G and Form 1 were observed, respectively. The forms obtained from the solubility experiments were Form 17, Form A and Form G, and Form 1.

The results of the solubility determination are summarized in Table 3. In most of the solvents, Compound 1A was sparingly or freely soluble except in water in which the free base was practically insoluble.

TABLE 3
Solubility of Compound 1A

| | Solubility | |
|---|---|---|
| Solvent | (mg/mL) | Form |
| Methanol | >463 | Not determined |
| Tetrahydrofuran | >429 | Not determined |
| Ethanol | >408 | Not determined |
| 2-Propanol | 229 | Form 17 |
| Acetone | 209 | Form A |
| Ethyl acetate | 127 | Form A |
| Chloroform | 70.6 | Form 17 |
| 1,4-Dioxane | 69.2 | Form G |
| Acetonitrile | 14.7 | Form 17 + Form A |
| Water | <0.1 | Form 1 |

### Example 3: Compound 1A—Anti-Precipitant Screening by Solvent Shift Method

The anti-precipitant screen by the solvent shift method was used for discovering polymers that can maintain the supersaturated state of Compound 1A in solution. Based on the results of this screen, polymers were selected for the development of the ASD.

Prior to the solvent shift experiments, the kinetic solubility of Compound 1A was tested in simulated intestinal fluid (SIF) and simulated gastric fluid (SGF) in order to choose the Compound 1A concentration suitable for the solvent shift experiments. Four different Compound 1A solutions (in DMSO) were prepared with concentrations of 60, 30, 15 and 5 mg/mL. Subsequently, 80 μL of these solutions were added to 4 mL of SGF and to 4 mL of SIF. In SGF precipitation was observed when using the solution with the highest concentration (60 mg/mL). The other three solutions did not precipitate indicating that in order to see an effect on the anti-precipitant properties of the polymers the initial Compound 1A concentration in DMSO has to be at least 60 mg/mL.

In case of SIF, the free base precipitated using all four concentrations. For the solvent shift experiments in SIF, Compound 1A concentration in DMSO of 5 mg/mL was used.

List of polymers used for the anti-precipitant studies by the solvent shift method in SIF and SGF is provided in Table 4. Differences in the list per medium are due to differences in solubility of the polymers in each medium.

TABLE 4
List of polymers used for the anti-precipitant studies:

| Polymers for SIF | Polymers for SGF |
|---|---|
| Hydroxyethyl cellulose (HEC) | Hydroxyethyl cellulose (HEC) |
| Hydroxypropyl methyl cellulose | Hydroxypropyl methyl cellulose |
| (HPMC) | (HPMC) |
| Hydroxypropyl cellulose (HPC) | Hydroxypropyl cellulose (HPC) |
| Eudragit L100 | Eudragit E100 |
| Polyethylene Glycol 6000 (PEG | Polyethylene Glycol 6000 (PEG |
| 6000) | 6000) |
| Polyvinylpyrrolidone K30 (PVP | Polyvinylpyrrolidone K30 (PVP |
| K30) | K30) |
| Poly(1-vinylpyrrolidone-co-vinyl | Poly(1-vinylpyrrolidone-co-vinyl |
| acetate) (PVP-VA) | acetate) (PVP-VA) |
| Pluronic F-68 | Pluronic F-68 |
| D-α-Tocopherol polyethylene | D-α-Tocopherol polyethylene |
| glycol 1000 succinate (TPGS) | glycol 1000 succinate (TPGS) |
| Carbopol ® 980 NF Polymer | Carbopol ® 980 NF Polymer |
| Soluplus ® | Soluplus ® |
| Povacoat Type MP | Povacoat Type MP |
| Kollicoat IR | Kollicoat IR |
| Sureteric | Methyl cellulose |

The polymer solutions were prepared by weighing 0.6 mg of polymer in an 8 mL glass vial. The polymer was dissolved in 4 mL of SIF or SGF (polymer concentration of 0.15 mg/mL). To the polymer solution 80 μL of Compound 1A solution in DMSO was added (5 mg/mL for the SIF experiments and 60 mg/mL for the SGF experiments, see Table 5). The vials were left to equilibrate at 25° C. with continuous stirring.

As a reference two control experiments were performed. In control experiment 1, crystalline Compound 1A was added to a pre-mixed solution of medium containing 2% DMSO. In control experiment 2, 80 μL of API in DMSO was added to the medium without polymer in the same manner as the experiments. After 0.5, 1, 2, 4 and 24 hours a small aliquot of mother liquor was taken and filtrated through a 0.45 μm PTFE filter to remove any particulate matter. The filtrate was diluted in acetonitrile, to prevent the sample from precipitation. The concentration of free base in solution was determined by HPLC analysis. The calibration curve was made from two independent stock solutions of the free base prepared in acetonitrile.

Table 5 describes preparation of SIF (pH 1.2) and SGF (pH 6.8) test solutions for the solvent shift method.

TABLE 5

| Test Solution | pH | |
|---|---|---|
| Simulated | | 7 mL of hydrochloric acid were diluted to 1000 |
| Gastric | | mL with water. Add and dissolve 2.0 grams of NaCl. |
| Fluid Test | | |
| Solution | | |
| Simulated | 6.8 | 6.8 grams of NaH₂PO₄ (anhydrous) were |
| Intestinal | | dissolved in 250 mL of water. |
| Fluid Test | | Then, 77 mL of 0.2N NaOH solution was added |
| Solution | | together with 500 mL of water. The pH was |
| | | adjusted to pH 6.8 with 0.2N NaOH. Water was |
| | | added till 1,000 mL |

TABLE 6
Details of the Compound 1A solutions in DMSO. The ratio
of Compound 1A:Polymer is based on the ratio of 80 μL API
solution in 4 mL polymer solution of 0.15 mg/mL.

| | | | Concentration | Concentration | |
|---|---|---|---|---|---|
| Stock | | | of API | determined | |
| for | API | DMSO | in DMSO | by HPLC | Ratio |
| medium | (mg) | (mL) | (mg/mL) | (mg/mL) | API:Polymer |
| SIF | 10.2 | 2.00 | 5.1 | 4.8 | 2:3 |
| SGF | 90.6 | 1.50 | 60.4 | 61.5 | 8:1 |

The experimental details and results of the experiments in SIF are described in Table 7, and of the experiments in SGF in Table 8. The concentration of Compound 1A in solution was determined by HPLC analysis after 0.5, 1, 2, 4 and 24 hours.

TABLE 7
Experimental conditions and results of the solvent shift precipitation experiments in SIF.

| | | Concentration (mg/mL) | | | | |
|---|---|---|---|---|---|---|
| Exp ID | Polymer | 0.5 h | 1 h | 2 h | 4 h | 24 h |
| REF101 | Hydroxyethyl cellulose (HEC) | 0.000 | <0.001 | <0.001 | <0.001 | <0.001 |
| REF102 | Hydroxypropyl methyl cellulose (HPMC) | 0.019 | 0.012 | 0.007 | 0.002 | <0.001 |
| REF103 | Hydroxypropyl cellulose (HPC) | 0.015 | 0.007 | 0.006 | <0.001 | <0.001 |
| REF104 | Eudragit L100 | 0.004 | 0.003 | <0.001 | <0.001 | 0.011 |
| REF105 | Polyethylene Glycol 6000 (PEG 6000) | 0.004 | <0.001 | <0.001 | <0.001 | <0.001 |
| REF106 | Polyvinylpyrrolidone K30 (PVP K30) | 0.012 | 0.004 | 0.008 | <0.001 | <0.001 |
| REF107 | Poly(1-vinylpyrrolidone-co-vinyl | 0.007 | 0.002 | 0.012 | 0.002 | <0.001 |
| | acetate) (PVP-VA) | | | | | |
| REF108 | Pluronic F-68 | <0.001 | <0.001 | <0.001 | <0.001 | <0.001 |
| REF109 | D-α-Tocopherol polyethylene | <0.001 | <0.001 | <0.001 | <0.001 | <0.001 |
| | glycol 1000 succinate | | | | | |
| REF110 | Carbopol ® 980 NF Polymer | <0.001 | 0.034 | 0.022 | <0.001 | <0.001 |
| REF111 | Soluplus ® | 0.027 | 0.012 | 0.021 | 0.015 | 0.013 |
| REF112 | Povacoat Type MP | 0.016 | <0.001 | <0.001 | <0.001 | <0.001 |
| REF113 | Kollicoat IR | <0.001 | <0.001 | <0.001 | <0.001 | <0.001 |
| REF114 | Sureteric | 0.001 | <0.001 | <0.001 | <0.001 | <0.001 |
| REF115 | No polymer - Control experiment - | <0.001 | <0.001 | <0.001 | <0.001 | <0.001 |
| | crystalline solid | | | | | |
| REF116 | No polymer - Crontol experiment - | 0.009 | <0.001 | 0.003 | 0.006 | <0.001 |
| | API in DMSO solution | | | | | |

TABLE 8
Experimental conditions and results of the solvent
shift precipitation experiments in SGF.

| | | Concentration (mg/mL) | | | | |
|---|---|---|---|---|---|---|
| Exp ID | Polymer | 0.5 h | 1 h | 2 h | 4 h | 24 h |
| REF117 | Hydroxyethyl cellulose (HEC) | 0.03 | 0.03 | 0.01 | 0.02 | 0.01 |
| REF118 | Hydroxypropyl methyl cellulose (HPMC) | 0.58 | 0.36 | 0.21 | 0.16 | 0.05 |
| REF119 | Hydroxypropyl cellulose (HPC) | 0.14 | 0.11 | 0.08 | 0.06 | 0.04 |
| REF120 | Eudragit E100 | 0.70 | 0.41 | 0.18 | 0.06 | 0.04 |
| REF121 | Polyethylene Glycol 6000 (PEG 6000) | 0.02 | 0.02 | 0.00 | 0.00 | 0.00 |
| REF122 | Polyvinylpyrrolidone K30 (PVP K30) | 0.20 | 0.09 | 0.03 | 0.01 | 0.01 |
| REF123 | Poly(1-vinylpyrrolidone-co-vinyl | 0.06 | 0.05 | 0.02 | 0.01 | 0.00 |
| | acetate) (PVP-VA) | | | | | |
| REF124 | Pluronic F-68 | 0.02 | 0.02 | 0.01 | 0.01 | 0.00 |
| REF125 | D-α-Tocopherol polyethylene | 0.04 | 0.04 | 0.04 | 0.03 | 0.02 |
| | glycol 1000 succinate | | | | | |
| REF126 | Carbopol ® 980 NF Polymer | 0.19 | 0.20 | 0.17 | 0.15 | <0.01 |
| REF127 | Soluplus ® | 0.48 | 0.49 | 0.48 | 0.44 | 0.07 |
| REF128 | Povacoat Type MP | 0.05 | 0.04 | 0.04 | 0.03 | 0.01 |
| REF129 | Kollicoat IR | 0.04 | 0.04 | 0.03 | 0.02 | 0.01 |
| REF130 | Methyl cellulose | 0.35 | 0.26 | 0.20 | 0.13 | 0.03 |
| REF131 | No polymer - Control experiment - | 0.02 | 0.04 | <0.01 | <0.01 | <0.01 |
| | crystalline solid | | | | | |
| REF132 | No polymer - Crontol experiment - | 0.02 | <0.01 | <0.01 | <0.01 | <0.01 |
| | API in DMSO solution | | | | | |

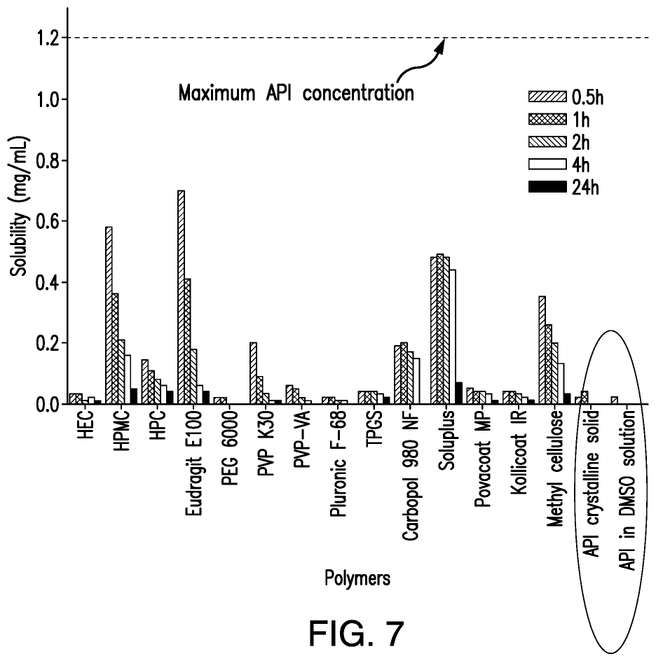

The results of the experiments performed in SGF polymer solutions are summarized in FIG. 7. The control experiments are highlighted with an oval. Compared to the control experiments, a significant solubility improvement was observed with most of the polymers. Higher super saturation and best anti-precipitant properties were observed with Soluplus® where after 4 hours the API concentration was maintained at 0.5 mg/mL. Other promising polymers were Carbopol®, HPMC, Eudragit E100 and methyl cellulose.

Using a SGF Carbopol® solution, a part of Compound precipitated quickly; however, Compound 1A concentration was maintained for 4 hours. In HPMC, Eudragit E100 and methyl cellulose solutions, Compound 1A slowly precipitated during the 24 hours test period. Based on these results it is assumed that Compound 1A has an affinity with these five polymers and therefore, they are promising polymers for further development of an amorphous dispersion

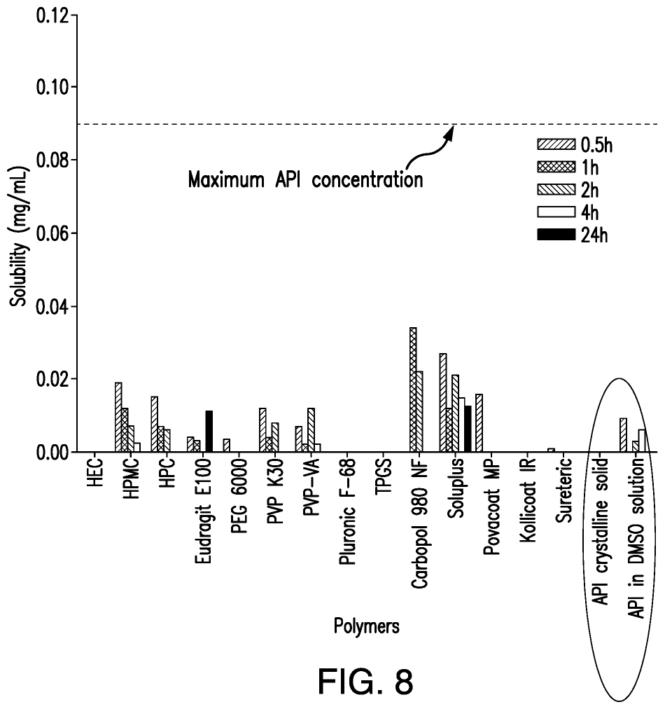

The results of the experiments performed in SIF are summarized in FIG. 8. The crystalline Compound 1A was practically insoluble in SIF as no Compound 1A was detected in solution. From the control experiment performed with Compound 1A in solution, Compound 1A could be detected after 0.5 hour, but not after 1 hour. After 2 and 4 hours, Compound 1A was detectable again. This phenomenon might be attributed to a solid form conversion or the result of the solubility being close to the detection limit. Due to the low solubility determined in the SIF solutions, the polymers for the preparation of the amorphous solid dispersions were chosen based on the SGF results.

### Example 4: Amorphous Solid Dispersion Screen for Compound 1A

The amorphous solid dispersion screen employed five polymers: Soluplus®, Carbopol®, Eudragit E100, HPMC and methyl cellulose. Three different solvent systems and three drug loads were used per polymer. For the preparation of the dispersions, solid Compound 1A and polymer were weighed in 8 mL glass vials. Solvent was added until a homogenous solution was obtained. Some mixtures were heated to ensure complete dissolutions. Control experiments containing only the Compound 1A (no excipient) were also prepared in different solvent mixtures. All the solutions were frozen in liquid nitrogen followed by drying using Crys Alpha 2-4 LO freeze-drier for 18 hours. The amorphous dispersions were all harvested and analyzed by HT-XRPD. No changes in HT-XRPD were observed after exposure to accelerated aging conditions (40° C./70% RH) for 4 weeks. The experimental conditions and results are reported in Table 9. In Table 9, ‘Am’ means amorphous. Control experiments were taken along for reference purposes (freeze-drying of Compound 1A in different solvent without polymer).

TABLE 9
Experimental conditions and results of the amorphous solid dispersion experiments

| | Mass | | Mass | Drug | | | Dissolve | | |
|---|---|---|---|---|---|---|---|---|---|
| | API | | Polymer | load | | Volume | at T | XRPD | |
| Exp ID | (mg) | Polymer | (mg) | (%) | Solvent | (mL) | (° C.) | Form | AAC |
| ASD1 | 5.2 | Carbopol ® 980 NF | 46.9 | 10 | Methanol/Water | 4.20 | 60 | Am glassy | Am glassy |
| | | | | | (90/10) | | | | |
| ASD2 | 5.5 | Carbopol ® 980 NF | 49.3 | 10 | Dichloromethane | 1.00 | 60 | Am | Am |
| ASD3 | 5.2 | Carbopol ® 980 NF | 47.1 | 10 | Tetrahydrofuran/ | 3.00 | 60 | Am | Am |
| | | | | | Water (50/50) | | | | |
| ASD4 | 12.5 | Carbopol ® 980 NF | 37.5 | 25 | Methanol/Water | 3.20 | 60 | Am glassy | Am glassy |
| | | | | | (90/10) | | | | |
| ASD5 | 12.5 | Carbopol ® 980 NF | 37.6 | 25 | Dichloromethane | 1.40 | 60 | Am | Am |
| ASD6 | 12.5 | Carbopol ® 980 NF | 37.5 | 25 | Tetrahydrofuran/ | 3.00 | 60 | Am | Am |
| | | | | | Water (50/50) | | | | |
| ASD7 | 25.0 | Carbopol ® 980 NF | 25.4 | 50 | Methanol/Water | 2.90 | 60 | Am | Am |
| | | | | | (90/10) | | | | |
| ASD8 | 25.0 | Carbopol ® 980 NF | 25.3 | 50 | Dichloromethane | 1.30 | 60 | Am | Form 1 |
| ASD9 | 25.1 | Carbopol ® 980 NF | 25.2 | 50 | Tetrahydrofuran/ | 3.00 | RT | Am | Am |
| | | | | | Water (50/50) | | | | |
| ASD10 | 5.3 | Soluplus ® | 47.3 | 10 | Dichloromethane | 1.00 | RT | Am glassy | Am glassy |
| ASD11 | 5.1 | Soluplus ® | 46.1 | 10 | Methanol/Water | 0.80 | RT | Form 1 | — |
| | | | | | (50/50) | | | | |
| ASD12 | 5.2 | Soluplus ® | 47.1 | 10 | Tetrahydrofuran/ | 1.00 | RT | Low | Am glassy |
| | | | | | Water (50/50) | | | crystalline | |
| ASD13 | 12.3 | Soluplus ® | 36.8 | 25 | Dichloromethane | 1.00 | RT | Am glassy | Am glassy |
| ASD14 | 12.5 | Soluplus ® | 37.6 | 25 | Methanol/Water | 0.80 | RT | Form 1 | Form 1 |
| | | | | | (50/50) | | | | |
| ASD15 | 12.5 | Soluplus ® | 37.6 | 25 | Tetrahydrofuran/ | 1.00 | RT | Am glassy | Am glassy |
| | | | | | Water (50/50) | | | | |
| ASD16 | 25.1 | Soluplus ® | 25.4 | 50 | Dichloromethane | 1.00 | RT | Am glassy | Am glassy |
| ASD17 | 25.3 | Soluplus ® | 25.2 | 50 | Methanol/Water | 1.00 | RT | Form 1 | Form 1 |
| | | | | | (50/50) | | | | |
| ASD18 | 25.1 | Soluplus ® | 25.2 | 50 | Tetrahydrofuran/ | 1.00 | RT | Am glassy | Am glassy |
| | | | | | Water (50/50) | | | | |
| ASD19 | 5.4 | Eudragit E100 | 48.8 | 10 | Methanol | 0.40 | RT | Am glassy | Am glassy |
| ASD20 | 5.0 | Eudragit E100 | 45.4 | 10 | Dichloromethane | 1.00 | RT | Am glassy | Am glassy |
| ASD21 | 5.0 | Eudragit E100 | 45.2 | 10 | Acetone/Ethanol | 1.00 | RT | Am glassy | Am glassy |
| | | | | | (80/20) | | | | |
| ASD22 | 12.5 | Eudragit E100 | 37.7 | 25 | Methanol | 0.40 | RT | Crystalline | Crystalline |
| ASD23 | 12.5 | Eudragit E100 | 37.8 | 25 | Dichloromethane | 1.00 | RT | Am glassy | Am glassy |
| ASD24 | 12.5 | Eudragit E100 | 37.6 | 25 | Acetone/Ethanol | 1.00 | RT | Am glassy | Am glassy |
| | | | | | (80/20) | | | | |
| ASD25 | 24.9 | Eudragit E100 | 24.9 | 50 | Methanol | 0.30 | RT | Crystalline | Crystalline |
| ASD26 | 25.1 | Eudragit E100 | 25.5 | 50 | Dichloromethane | 1.00 | RT | Am glassy | Am glassy |
| ASD27 | 25.1 | Eudragit E100 | 25.2 | 50 | Acetone/Ethanol | 1.00 | RT | Am | — |
| | | | | | (80/20) | | | | |
| ASD28 | 4.9 | HPMC | 44.3 | 10 | Acetone/Methanol | 0.40 | RT | Am | Am |
| | | | | | (80/20) | | | | |
| ASD29 | 4.9 | HPMC | 44.4 | 10 | Dichloromethane/ | 0.40 | RT | Am glassy | Am glassy |
| | | | | | Ethanol (50/50) | | | | |
| ASD30 | 5.1 | HPMC | 45.6 | 10 | Tetrahydrofuran/ | 1.00 | RT | Am | Am |
| | | | | | Water (50/50) | | | | |
| ASD31 | 12.6 | HPMC | 37.6 | 25 | Acetone/Methanol | 0.40 | RT | Am glassy | Am glassy |
| | | | | | (80/20) | | | | |
| ASD32 | 12.5 | HPMC | 37.5 | 25 | Dichloromethane/ | 0.40 | RT | Am glassy | Am glassy |
| | | | | | Ethanol (50/50) | | | | |
| ASD33 | 12.6 | HPMC | 38.0 | 25 | Tetrahydrofuran/ | 1.00 | RT | Am | Am |
| | | | | | Water (50/50) | | | | |
| ASD34 | 24.9 | HPMC | 25.5 | 50 | Acetone/Methanol | 0.80 | RT | Am | Am |
| | | | | | (80/20) | | | | |
| ASD35 | 24.9 | HPMC | 25.9 | 50 | Dichloromethane/ | 0.40 | RT | Am glassy | Am glassy |
| | | | | | Ethanol (50/50) | | | | |
| ASD36 | 25.2 | HPMC | 26.0 | 50 | Tetrahydrofuran/ | 1.00 | RT | Am | Am |
| | | | | | Water (50/50) | | | | |
| ASD37 | 5.1 | Methyl cellulose | 45.8 | 10 | Dichloromethane/ | 3.70 | 60 | Am glassy | Am glassy |
| | | | | | Methanol (50/50) | | | | |
| ASD38 | 5.1 | Methyl cellulose | 45.6 | 10 | Chloroform/Ethanol | 1.50 | 60 | Am glassy | Am glassy |
| | | | | | (50/50) | | | | |
| ASD39 | 5.0 | Methyl cellulose | 45.1 | 10 | Tetrahydrofuran/ | 4.00 | RT | Am | Am |
| | | | | | Water (50/50) | | | | |
| ASD40 | 12.4 | Methyl cellulose | 37.4 | 25 | Dichloromethane/ | 3.20 | 60 | Am glassy | Am glassy |
| | | | | | Methanol (50/50) | | | | |
| ASD41 | 12.6 | Methyl cellulose | 37.6 | 25 | Chloroform/Ethanol | 4.00 | RT | Am | Am |
| | | | | | (50/50) | | | | |
| ASD42 | 12.5 | Methyl cellulose | 37.5 | 25 | Tetrahydrofuran/ | 4.00 | RT | Am | Am |
| | | | | | Water (50/50) | | | | |
| ASD43 | 25.0 | Methyl cellulose | 25.0 | 50 | Dichloromethane/ | 3.20 | 60 | Am glassy | Am glassy |
| | | | | | Methanol (50/50) | | | | |
| ASD44 | 25.1 | Methyl cellulose | 25.1 | 50 | Chloroform/Ethanol | 2.00 | 60 | Am glassy | Am glassy |
| | | | | | (50/50) | | | | |
| ASD45 | 25.1 | Methyl cellulose | 25.6 | 50 | Tetrahydrofuran/ | 3.00 | RT | Am | Am |
| | | | | | Water (50/50) | | | | |
| ASD100 | 50.6 | — | — | 100 | Methanol/Water | 1.00 | RT | Low | Form 1 |
| | | | | | (90/10) | | | crystalline | |
| ASD101 | 50.0 | — | — | 100 | Dichloromethane | 4.00 | RT | Am | Form 1 |
| ASD102 | 49.4 | — | — | 100 | Tetrahydrofuran/ | 0.80 | RT | Am | Form 1 |
| | | | | | Water (50/50) | | | | |
| ASD103 | 50.3 | — | — | 100 | Methanol/Water | 6.60 | RT | Form 1 | Form 1 |
| | | | | | (50/50) | | | | |
| ASD104 | 50.6 | — | — | 100 | Methanol | 1.80 | RT | Low | Form 1 |
| | | | | | | | | crystalline | |
| ASD105 | 51.5 | — | — | 100 | Acetone/Ethanol | 1.00 | RT | Am | Form 1 |
| | | | | | (80/20) | | | | |
| ASD106 | 50.4 | — | — | 100 | Acetone/Methanol | 0.80 | RT | Low | Form 1 |
| | | | | | (50/50) | | | crystalline | |
| ASD107 | 51.5 | — | — | 100 | Dichloromethane/ | 0.80 | RT | Form 1 | Form 1 |
| | | | | | Ethanol (50/50) | | | | |

With all five polymers, amorphous solid dispersions were obtained. With Soluplus® and Eudragit E100 all the dispersions appeared to be glassy or films. For that reason, none of these dispersions were selected for further investigations.

The selection of amorphous dispersion for further characterization was based on the appearance of the dispersion before and after exposure to accelerated aging condition. The stable amorphous solid dispersions were further characterized by DSC and TGMS.

The amorphous solid dispersions prepared with Carbopol®, HPMC and methyl cellulose were mostly powdery. For these polymers, the best dispersions were obtained from THF/water (50/50) with all three drug loadings (5, 20 and 50%). No significant changes were observed during the stability study. In most of the cases, the dispersions were physically stable and remained amorphous.

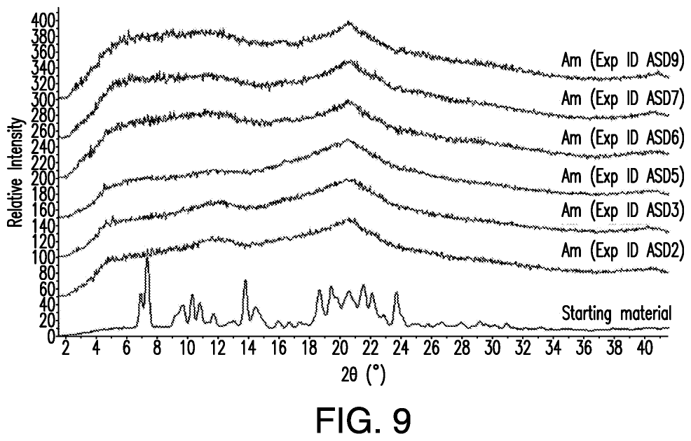

FIG. 9 provides an overlay of HT-XRPD patterns of crystalline Compound 1A and a solid dispersion prepared with Carbopol® 980 NF, 10% drug load in DCM (Exp. ID ASD2), 10% drug load in THF/water (50/50) (Exp. ID ASD3), 25% drug load in DCM (Exp. ID ASD5), 25% drug load in THF/water (50/50) (Exp. ID ASD6), 50% drug load in methanol/water (90/10) (Exp. ID ASD7) and 50% drug load in THF/water (50/50) (Exp. ID ASD9).

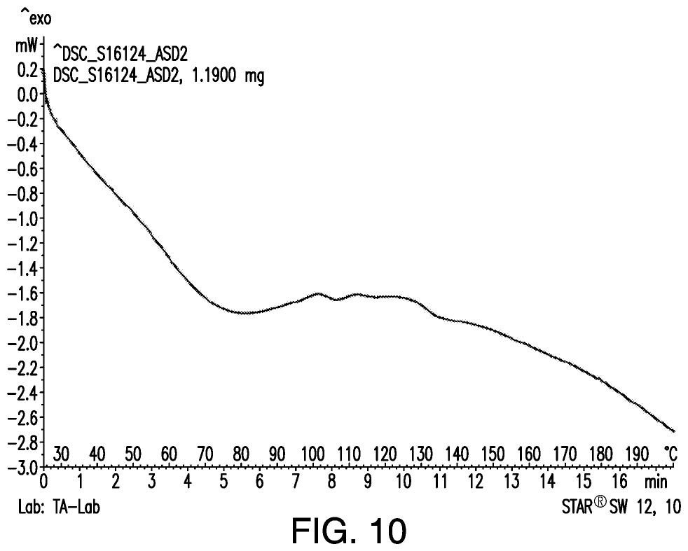

FIG. 10 provides a DSC spectrum (with a heating rate of 10° C./min and a pierced pan) of the amorphous solid dispersion prepared with Carbopol® 980 NF, 10% drug load in DCM (Exp. ID ASD2). No melting event was recorded, confirming the lack of crystalline material.

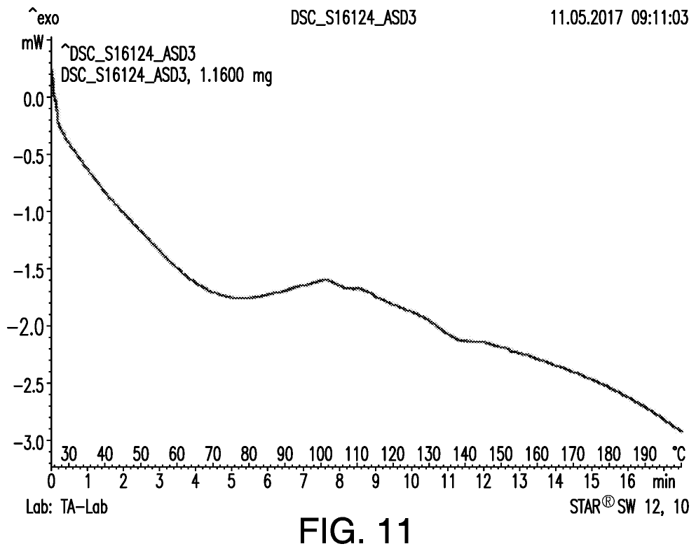

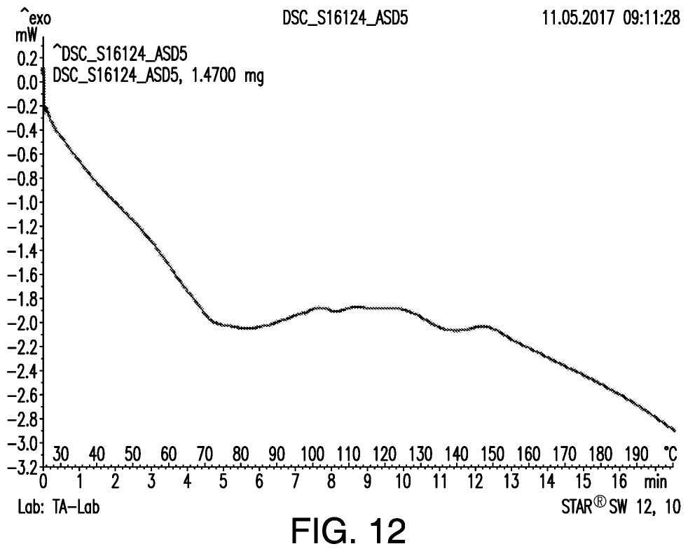

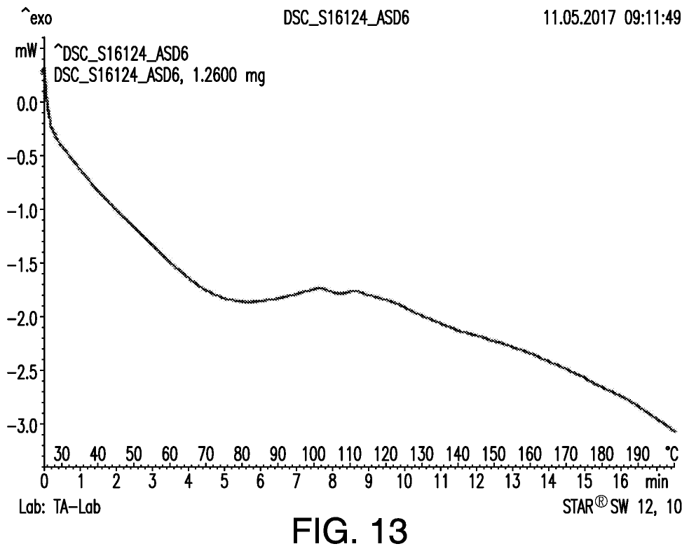

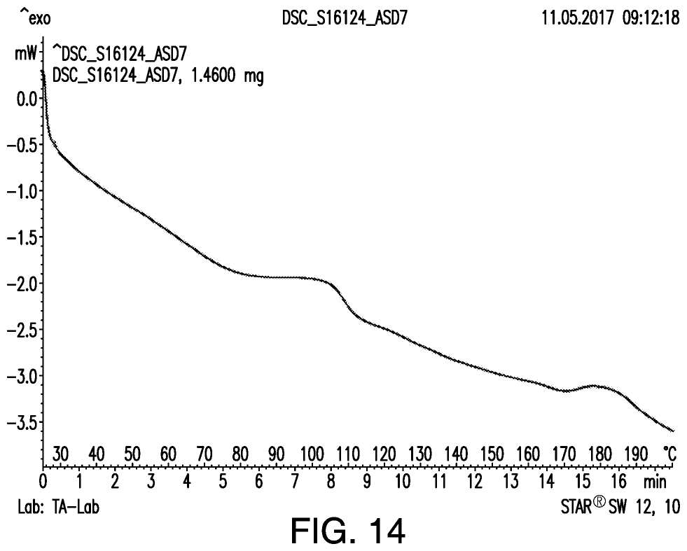

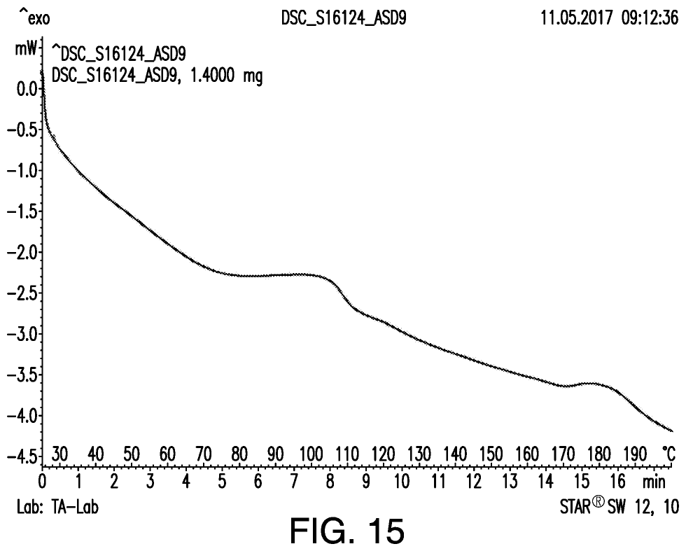

FIG. 11 provides a DSC spectrum (with a heating rate of 10° C./min and a pierced pan) of the amorphous solid dispersion prepared with Carbopol® 980 NF, 10% drug load in THF/water (50/50) (Exp. ID ASD3). No melting event was recorded, confirming the lack of crystalline material. FIG. 12 provides a DSC spectrum (with a heating rate of 10° C./min and a pierced pan) of the amorphous solid dispersion prepared with Carbopol® 980 NF, 25% drug load in DCM (Exp. ID ASD5). No melting event was recorded, confirming the lack of crystalline material. FIG. 13 provides a DSC spectrum (with a heating rate of 10° C./min and a pierced pan) of the amorphous solid dispersion prepared with Carbopol® 980 NF, 25% drug load in THF/water (50/50) (Exp. ID ASD6). No melting event was recorded. FIG. 14 provides a DSC spectrum (with a heating rate of 10° C./min and a pierced pan) of the amorphous solid dispersion prepared with Carbopol® 980 NF, 50% drug load in methanol/water (90/10) (Exp. ID ASD7). No melting event was recorded, confirming the lack of crystalline material. FIG. 15 provides a DSC spectrum (with a heating rate of 10° C./min) of the amorphous solid dispersion prepared with Carbopol® 980 NF, 50% drug load in THF/water (50/50) (Exp. ID ASD9). No melting event was recorded, confirming the lack of crystalline material.

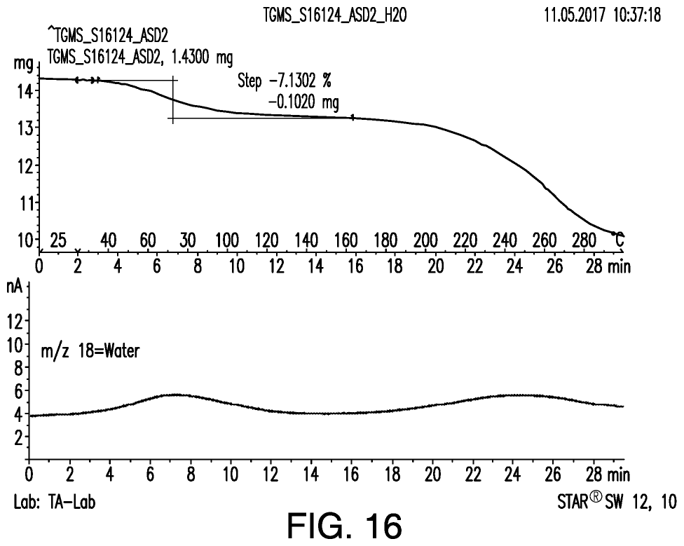

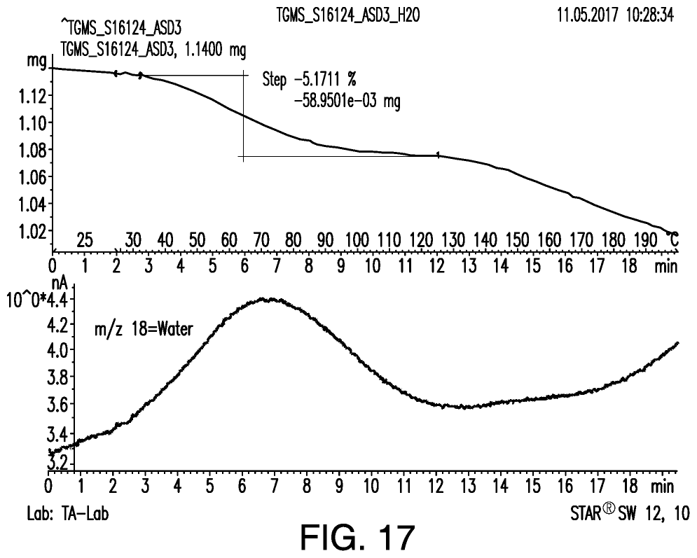

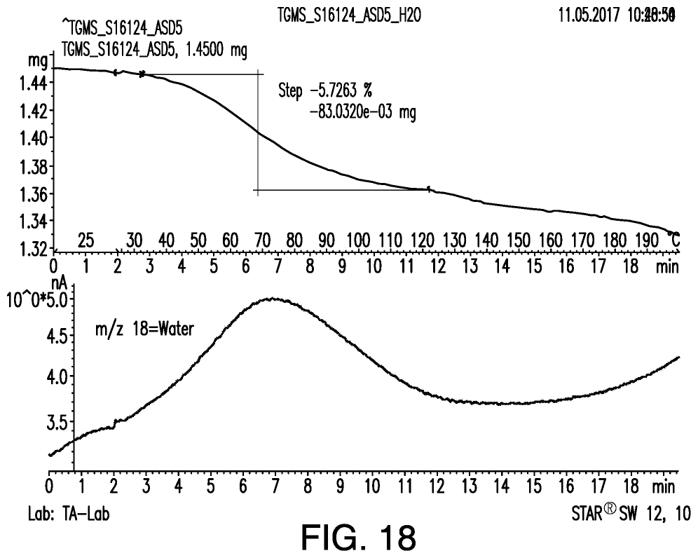

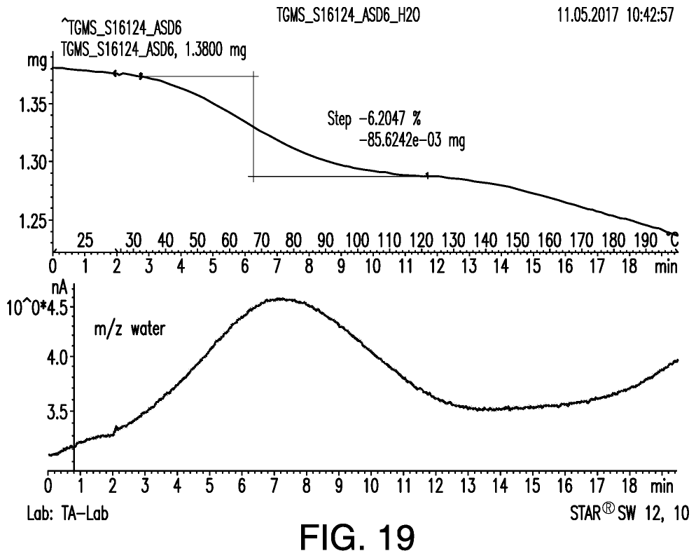

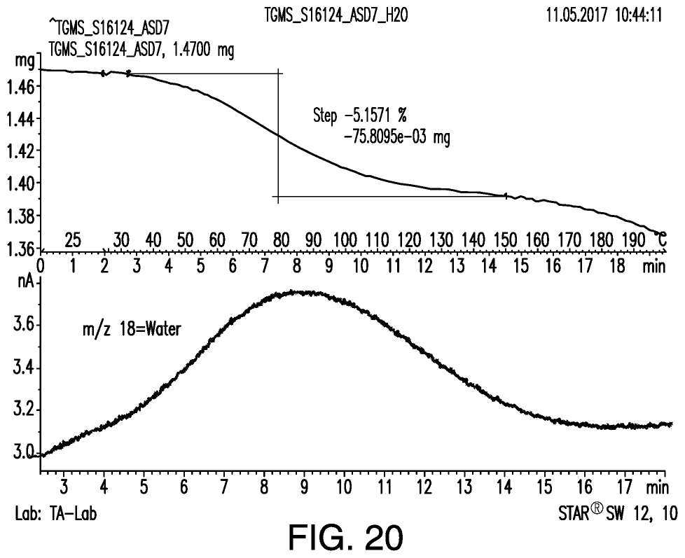

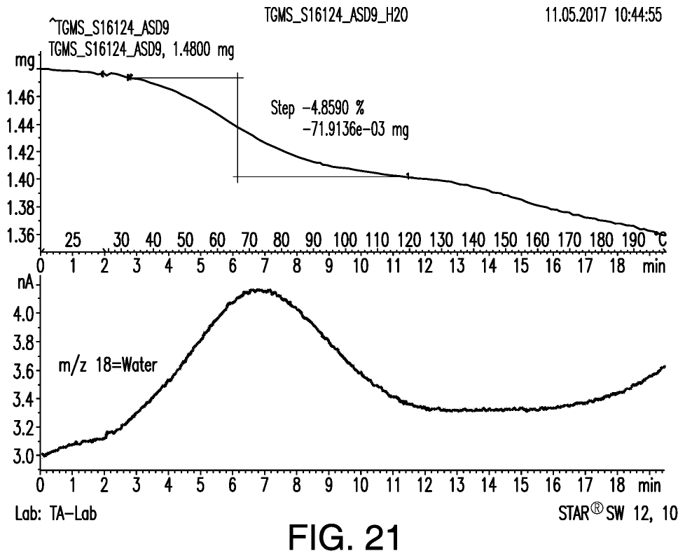

FIG. 16 provides a TGMS spectrum (with a heating rate of 10° C./min) of the amorphous solid dispersion prepared with Carbopol® 980 NF, 10% drug load in DCM (Exp. ID ASD2). The TGMS signal shows a mass loss of 7.1%, attributed to water prior to the thermal decomposition. FIG. 17 provides a TGMS spectrum (with a heating rate of 10° C./min) of the amorphous solid dispersion prepared with Carbopol® 980 NF, 10% drug load in THF/water (50/50) (Exp. ID ASD3). The TGMS signal shows a mass loss of 5.2% attributed to water prior to the thermal decomposition. FIG. 18 provides a TGMS spectrum (with a heating rate of 10° C./min) of the amorphous solid dispersion prepared with Carbopol® 980 NF, 25% drug load in DCM (Exp ID ASD5). The TGMS signal shows a mass loss of 5.7% attributed to water prior to thermal decomposition. FIG. 19 provides a TGMS spectrum (with a heating rate of 10° C./min) of the amorphous solid dispersion prepared with Carbopol® 980 NF, 25% drug load in THF/water (50/50) (Exp. ID ASD6). The TGMS signal shows a mass loss of 5.7% attributed to water prior to the thermal decomposition. FIG. 20 provides a TGMS spectrum (with a heating rate of 10° C./min) of the amorphous solid dispersion prepared with Carbopol® 980 NF, 50% drug load in methanol/water (90/10) (Exp. ID ASD7). The TGMS signal shows a mass loss of 5.2% attributed to water prior to the thermal decomposition. FIG. 21 provides a TGMS spectrum (with a heating rate of 10° C./min) of the amorphous solid dispersion prepared with Carbopol® 980 NF, 50% drug load in THF/water (50/50) (Exp. ID ASD9). The TGMS signal shows a mass loss of 4.9% attributer to water prior to the thermal decomposition.

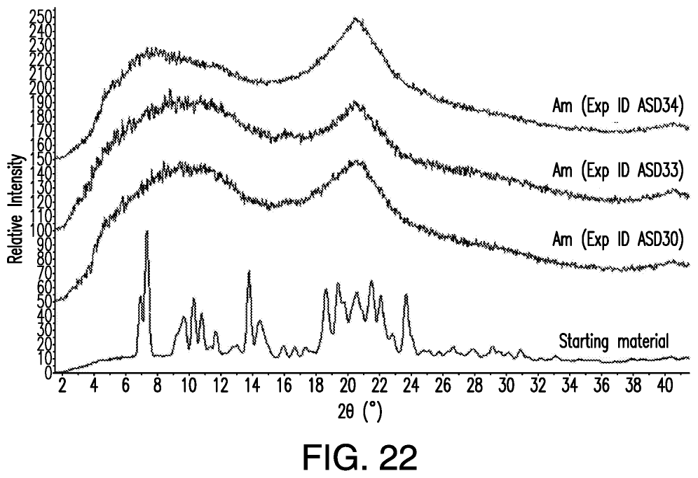

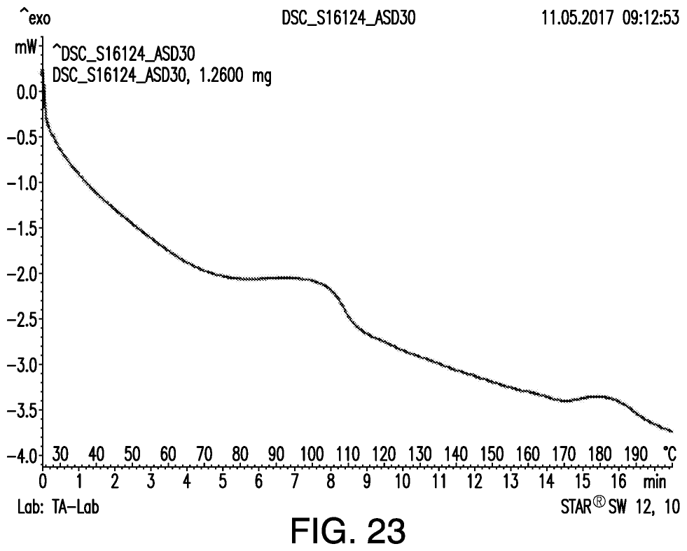

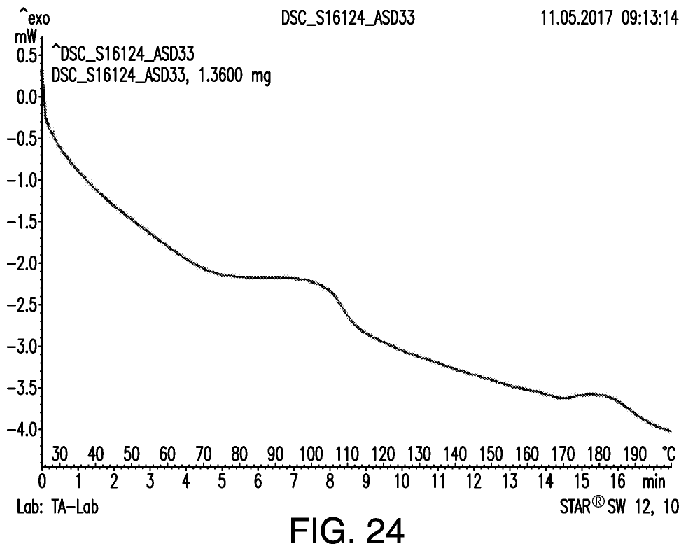

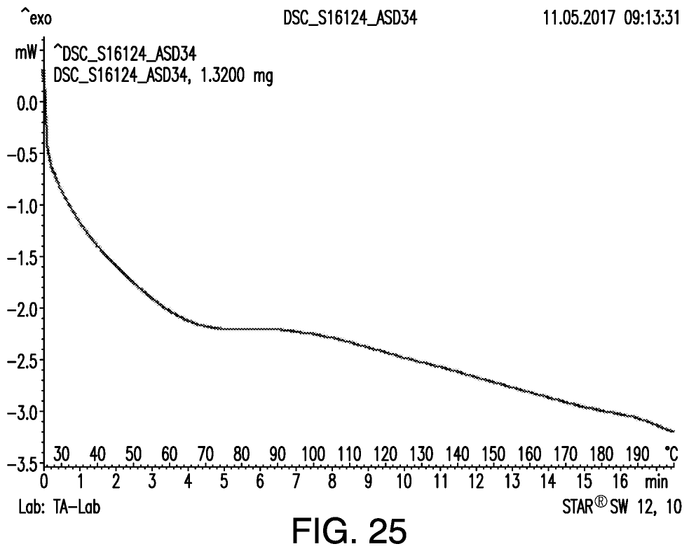

FIG. 22 provides an overlay of HT-XRPD patterns (from bottom to top): Compound 1A and amorphous solid dispersions prepared with HPMC, 10% drug load in THF/water (50/50) (Exp. ID ASD30), 25% drug load in THF/water (50/50) (Exp. ID ASD33) and 50% drug load in acetone/methanol (80/20) (Exp. ID ASD34). No changes in HT-XRPD were observed after exposure to accelerated aging conditions (40° C./70% RH) for 4 weeks. FIG. 23 provides a DSC spectrum (with a heating rate of 10° C./min and a pierced pan) of the amorphous solid dispersions prepared with HPMC, 10% drug load in THF/water (50/50) (Exp. ID ASD30). No melting event was recorded, confirming the lack of crystalline material. FIG. 24 provides a DSC spectrum (with a heating rate of 10° C./min and a pierced pan) of the amorphous solid dispersions prepared with HPMC, 25% drug load in THF/water (50/50) (Exp. ID ASD33). No melting event was recorded, confirming the lack of crystalline material. FIG. 25 provides a DSC spectrum (with a heating rate of 10° C./min and a pierced pan) of the amorphous solid dispersions prepared with HPMC, 50% drug load in acetone/methanol (80/20) (Exp. ID ASD34). No melting event was recorded, confirming the lack of crystalline material.

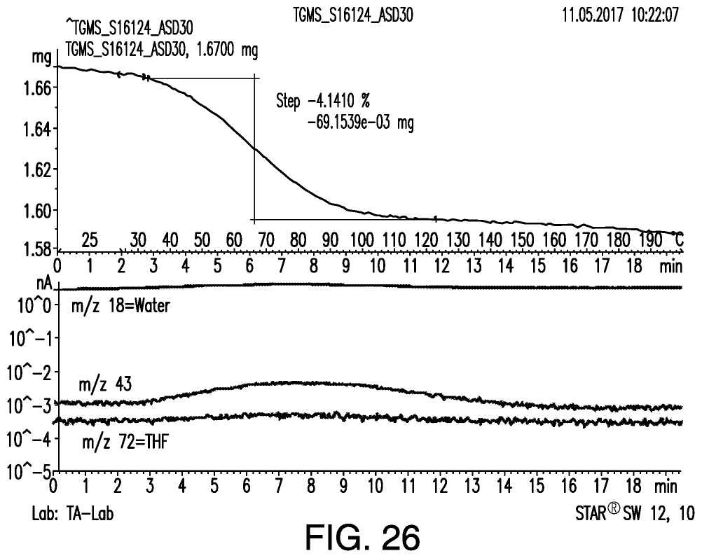

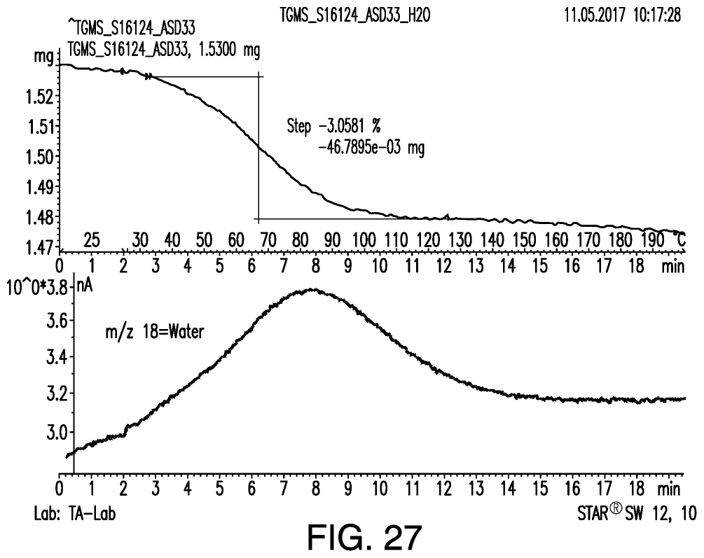

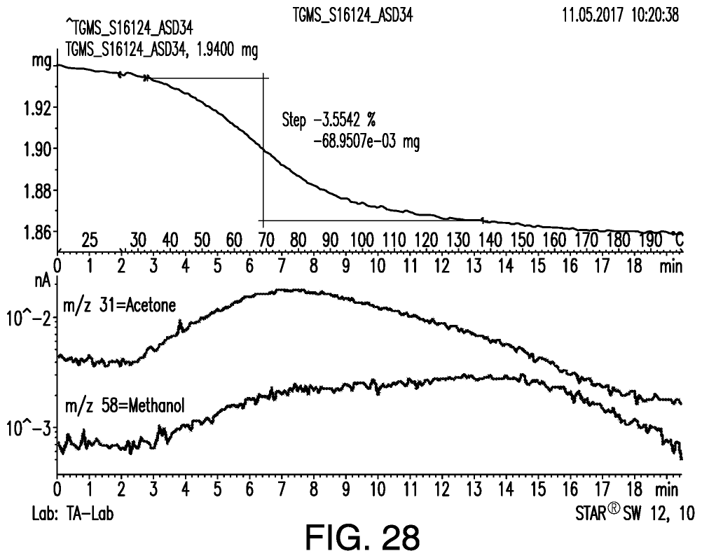

FIG. 26 provides a TGMS spectrum (with a heating rate of 10° C./min) of the amorphous solid dispersions prepared with HPMC, 10% drug load in THF/water (50/50) (Exp. ID ASD30). The TGMS signal shows a mass loss of 4.1% attributed to water prior to thermal decomposition. FIG. 27 provides a TGMS spectrum (with a heating rate of 10° C./min) of the amorphous solid dispersions prepared with HPMC, 25% drug load in THF/water (50/50) (Exp. ID ASD33). The TGMS signal shows a mass loss of 3.1% attributed to water prior to thermal decomposition. FIG. 28 provides a TGMS spectrum (with a heating rate of 10° C./min) of the amorphous solid dispersions prepared with HPMC, 50% drug load in acetone/methanol (80/20) (Exp. ID ASD34). The TGMS signal shows a mass loss of 3.6% attributed to methanol and acetone prior to thermal decomposition.

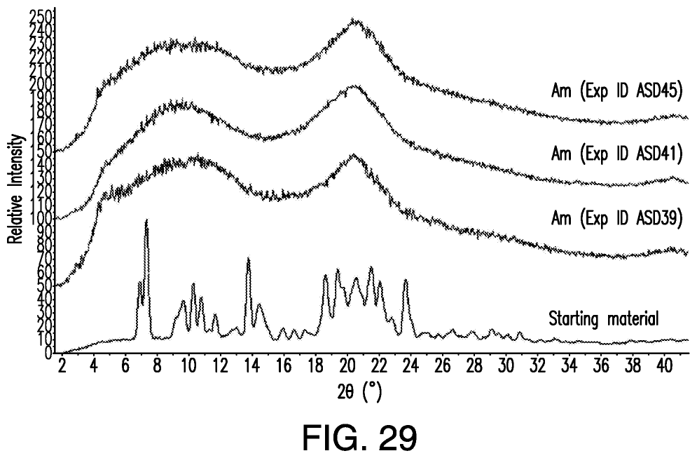

FIG. 29 provides an overlay of HT-XRPD patterns (from bottom to top): crystalline Enasidenib free base and amorphous solid dispersions prepared with methyl cellulose, 10% drug load in THF/water (50/50) (Exp. ID ASD39), 25% drug load in chloroform/ethanol (50/50) (Exp. ID ASD41) and 50% drug load in THF/water (50/50) (Exp. ID ASD45). No changes in HT-XRPD were observed after exposure to accelerated aging conditions (40° C./70% RH) for 4 weeks.

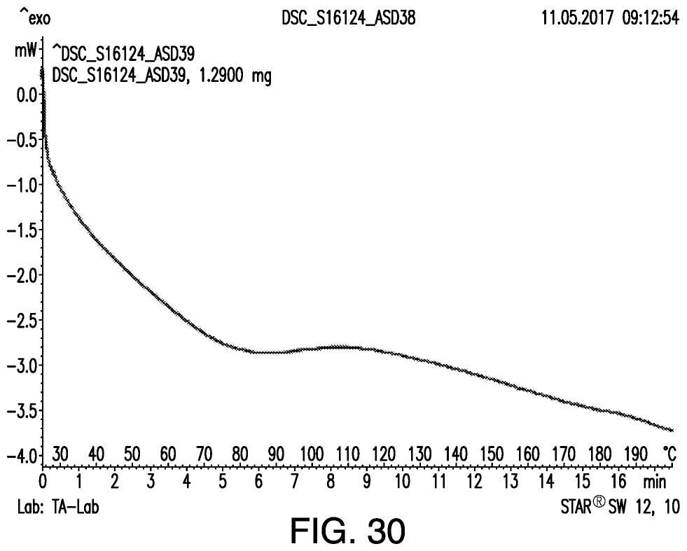

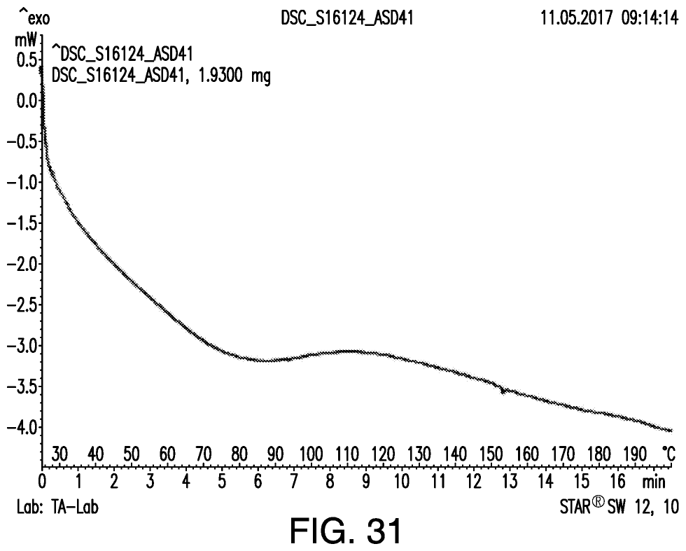

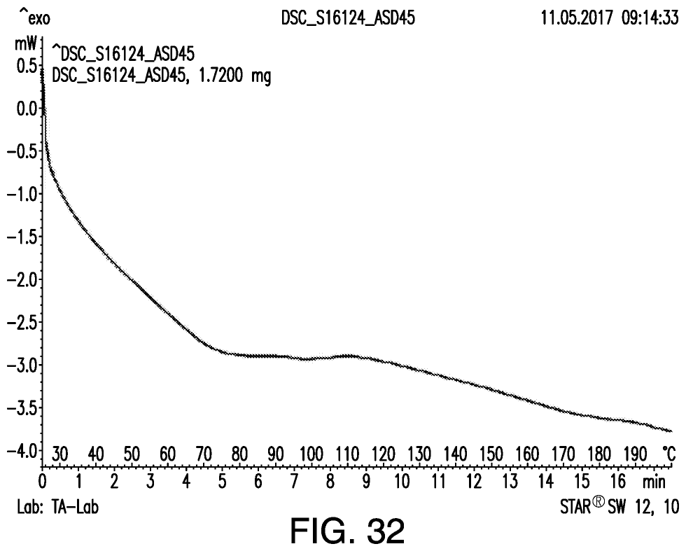

FIG. 30 provides a DSC spectrum (with a heating rate of 10° C./min and a pierced pan) of the amorphous solid dispersion prepared with methyl cellulose, 10% drug load in THF/water (50/50) (Exp. ID ASD39). No melting event was recorded, confirming the lack of crystalline material. FIG. 31 provides a DSC spectrum (with a heating rate of 10° C./min and a pierced pan) of the amorphous solid dispersion prepared with methyl cellulose, 25% drug load in chloroform/ethanol (50/50) (Exp. ID ASD41). No melting event was recorded, confirming the lack of crystalline material. FIG. 32 provides a DSC spectrum (with a heating rate of 10° C./min and a pierced pan) of the amorphous solid dispersion prepared with methyl cellulose, 50% drug load in THF/water (50/50) (Exp. ID ASD45). No melting event was recorded, confirming the lack of crystalline material.

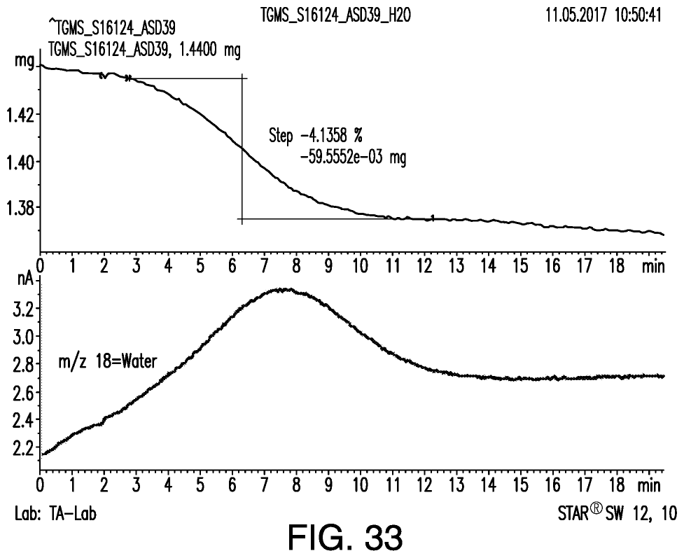

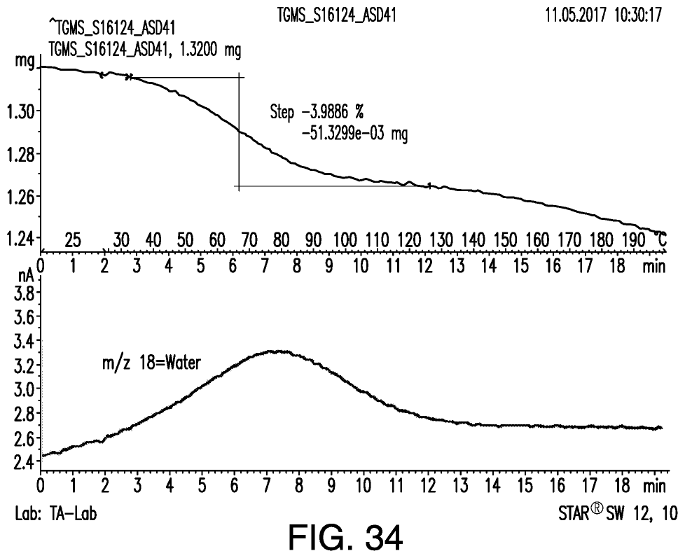

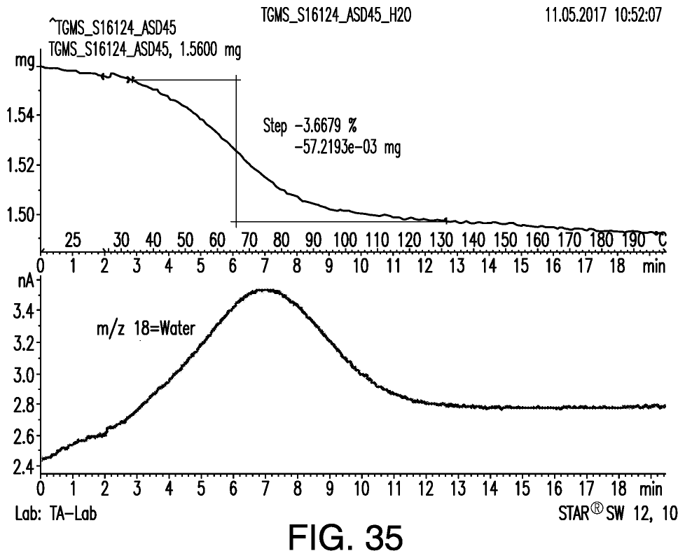

FIG. 33 provides a TGMS spectrum (with a heating rate of 10° C./min) of the amorphous solid dispersion prepared with methyl cellulose, 10% drug load in THF/water (50/50) (Exp. ID ASD39). The TGMS signal shows a mass loss of 4.1% attributed to water prior to the thermal decomposition. FIG. 34 provides a TGMS spectrum (with a heating rate of 10° C./min) of the amorphous solid dispersion prepared with methyl cellulose, 25% drug load in chloroform/ethanol (50/50) (Exp. ID ASD41). The TGMS signal shows a mass loss of 3.9% attributed to water prior to the thermal decomposition. FIG. 35 provides a TGMS spectrum (with a heating rate of 10° C./min) of the amorphous solid dispersion prepared with methyl cellulose, 50% drug load in THF/water (50/50) (Exp. ID ASD45). The TGMS signal shows a mass loss of 3.7% attributed to water prior to the thermal decomposition.

None of the DSC analyses indicated that crystalline material was present or that temperature induced crystallization occurred in the dispersions. Most samples contained 4-6% water. From these thermal analyses, it became apparent that even after the solvent loss, crystallization of Compound 1A is not occurring; therefore, it might be possible to reduce the solvent content by additional drying.

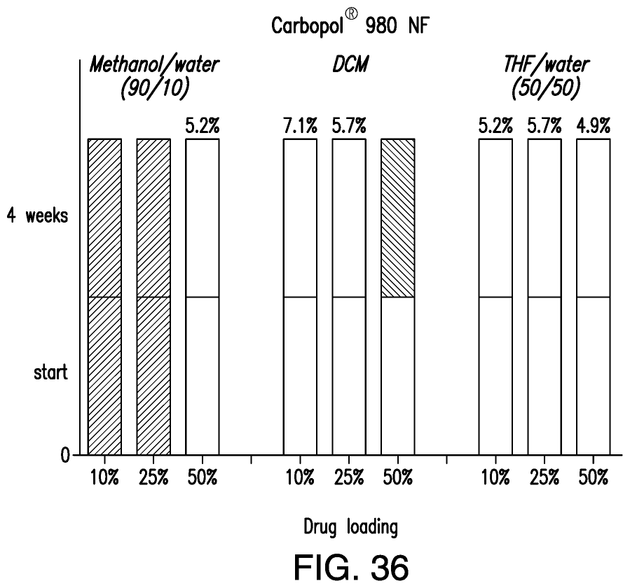

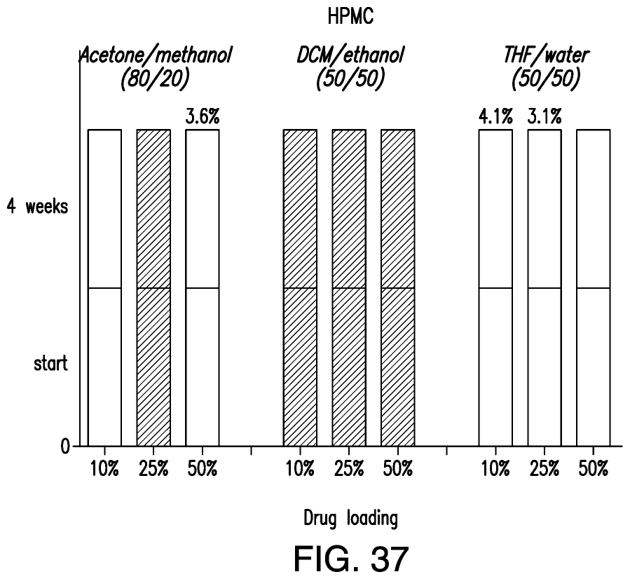

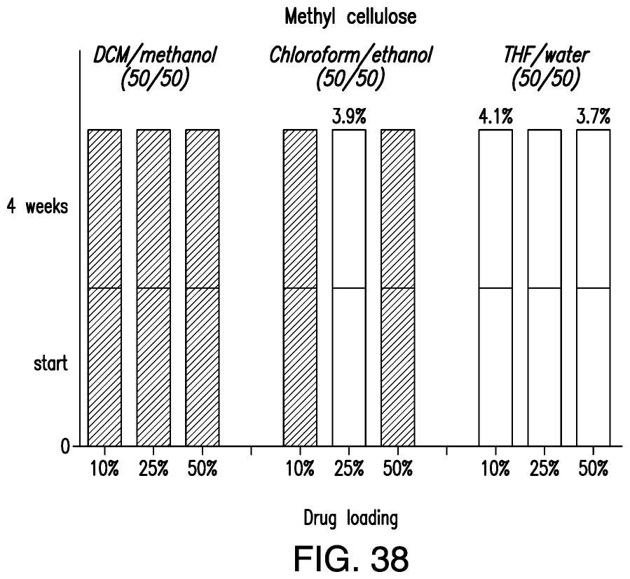

The graphical representation of the amorphous dispersions including the loss on drying determined by TGMS are presented in FIG. 36 for Carbopol®, FIG. 37 for HPMC and FIG. 38 for methyl cellulose. In FIGS. 36-38, each bar represents a solid dispersion.

FIG. 36 provides a graphical representation of the solid dispersion prepared with Carbopol® 980 NF in three solvents systems before and after stability for 4 weeks. The dispersions with 50% drug load in methanol/water (90/10), 10% and 25% drug loads in DCM and 10%, 25% and 50% drug loads in THF/water (50/50) were amorphous powder. The dispersions with 10% and 25% drug load in methanol/water (90/10) were glassy. The dispersion with 50% drug load in DCM had traces of the crystalline API as seen in the XRPD diffractogram. The loss on drying determined by TGMS analysis on the amorphous solid dispersions is presented above each bar.

FIG. 37 provides a graphical representation of the solid dispersion prepared with HPMC in three solvents systems before and after stability for 4 weeks. The dispersions with 10% and 50% drug load in acetone/methanol (80/20), and 10%, 25% and 50% drug loads in THF/water (50/50) were amorphous powder. The dispersions with 25% drug load in acetone/methanol (80/20), and 10%, 25% and 50% drug loads in DCM/methanol (50/50) were glassy. The loss on drying determined by TGMS analysis on the amorphous solid dispersions is presented above each bar.

FIG. 38 provides a graphical representation of the solid dispersion prepared with methyl cellulose in three solvents systems before and after stability for 4 weeks. The dispersions with 25% drug load in chloroform/ethanol (50/50), and 10%, 25% and 50% drug loads in THF/water (50/50) were amorphous powder. The dispersions with 10%, 25% and 50% drug loads in DCM/methanol (50/50), and 10% and 50% drug load in chloroform/ethanol (50/50) were glassy. The loss on drying determined by TGMS analysis is presented above each bar.

### Example 5: Solubility Determination of Amorphous Solid Dispersions of Compound 1A

The thermodynamic solubility of the dispersions was determined in water, SIF and SGF at 37° C. SIF and SGF were prepared according to the description in the USP test solutions and were prepared without enzymes. The solubility of the crystalline starting material was determined as reference. Additionally the solubility of three batches of amorphous API (without excipient) was determined.

The solubility of Compound 1A solid dispersions (Table 10) was compared to the crystalline and amorphous Compound 1A. The control experiments in Table 10 (amorphous Compound 1A without counter-ion) are Exp. ID ASD101, ASD102, ASD105.

TABLE 10
Conditions of the amorphous solid dispersions
that were used for the solubility studies.

| | | Drug | |
|---|---|---|---|
| Exp ID | Excipient | load (%) | Solvent |
| ASD2 | Carbopol ® 980 NF | 10 | DCM |
| ASD3 | Carbopol ® 980 NF | 10 | THF/water (50/50) |
| ASD5 | Carbopol ® 980 NF | 25 | DCM |
| ASD6 | Carbopol ® 980 NF | 25 | THF/water (50/50) |
| ASD7 | Carbopol ® 980 NF | 50 | MeOH/water (90/10) |
| ASD9 | Carbopol ® 980 NF | 50 | THF/water (50/50) |
| ASD30 | HPMC | 10 | THF/water (50/50) |
| ASD33 | HPMC | 25 | THF/water (50/50) |
| ASD34 | HPMC | 50 | Acetone/MeOH (80/20) |
| ASD39 | Methyl cellulose | 10 | THF/water (50/50) |
| ASD41 | Methyl cellulose | 25 | Chloroform/EtOH (50/50) |
| ASD45 | Methyl cellulose | 50 | THF/water (50/50) |
| ASD101 | — | — | Dichloromethane |
| ASD102 | — | — | Tetrahydrofuran/Water |
| | | | (50/50) |
| ASD105 | — | — | Acetone/Ethanol (80/20) |

The solubility of Compound 1A released from the dispersions was determined at 37° C. in water, SGF and SIF. For the solubility determination, suspensions of Compound 1A and dispersions were prepared in 3 mL aqueous medium. The suspensions were incubated at 37° C. under continuous stirring. Samples were taken after 0.5, 1 and 2 hours, filtered over a PTFE spin filter and the concentration of Enasidenib in solution was determined by HPLC analysis. In the next sections the solubility results are discussed per aqueous media.

The amorphous solid dispersions were weighed in 8 mL glass vials and 3 mL of the medium was added. The suspensions were incubated at 37° C. under continuous stirring. The solubility was determined after 0.5, 1 and 2 hours. At each equilibration time, an aliquot of the sample was filtered through a 0.45 μm PTFE filter to remove any particulate matters. The filtrate was diluted in acetonitrile and analyzed by HPLC to determine the concentration of solute. The pH was recorded at the start and at the end of the equilibration time. The experimental details and results are reported in Table 11. The concentration of API in solution was determined by HPLC analysis after 0.5, 1 and 2 hours.

TABLE 11
Experimental conditions and results of the solubility determination on the
amorphous solid dispersions with Carbopol ®, HPMC and methyl cellulose

| | | | | | Mass | | | | | | |
|---|---|---|---|---|---|---|---|---|---|---|---|
| | Solid | | Drug | Dispersion | dispersion | | | Concentration API | | | |
| Exp ID | material | Excipient | load | Solvent | (mg) | Medium | pH | (μg/mL) | | | pH |
| QSA44 | SM | — | — | — | 12.4 | Water | 7.6 | 1.0 | 0.4 | <0.1 | 5.6 |
| QSA45 | | | | | 11.2 | SIF | 6.7 | 0.3 | <0.1 | <0.1 | 6.6 |
| QSA46 | | | | | 11.0 | SGF | 1.3 | 75.8 | 41.3 | 19.4 | 1.4 |
| QSA50 | ASD2 | Carbopol ® | 10 | Dichloromethane | 9.6 | Water | 4.2 | 0.4 | 0.6 | 0.9 | 3.9 |
| QSA51 | | | | | 9.8 | SIF | 6.1 | <0.1 | <0.1 | <0.1 | 5.8 |
| QSA52 | | | | | 10.2 | SGF | 1.3 | 77.9 | 112.6 | 117.5 | 1.3 |
| QSA53 | ASD3 | | 10 | Tetrahydrofuran/ | 11.4 | Water | 4.4 | 0.6 | 1.1 | 1.2 | 3.9 |
| QSA54 | | | | Water (50/50) | 12.0 | SIF | 6.5 | 0.5 | <0.1 | <0.1 | 5.8 |
| QSA55 | | | | | 10.1 | SGF | 1.3 | 81.6 | 84.6 | 64.5 | 1.3 |
| QSA56 | ASD5 | | 25 | Dichloromethane | 9.8 | Water | 4.6 | 0.4 | 0.6 | 0.7 | 4.2 |
| QSA57 | | | | | 10.8 | SIF | 6.2 | <0.1 | <0.1 | <0.1 | 5.8 |
| QSA58 | | | | | 10.0 | SGF | 1.3 | 202.3 | 127.7 | 143.1 | 1.3 |
| QSA59 | ASD6 | | 25 | Tetrahydrofuran/ | 9.4 | Water | 4.8 | 0.2 | 0.9 | 0.5 | 4.2 |
| QSA60 | | | | Water (50/50) | 9.5 | SIF | 6.5 | <0.1 | <0.1 | <0.1 | 5.7 |
| QSA61 | | | | | 9.9 | SGF | 1.3 | 68.6 | 132.7 | 129.8 | 1.3 |
| QSA62 | ASD7 | | 50 | Methanol/Water | 9.0 | Water | 4.9 | 0.9 | <0.1 | <0.1 | 4.7 |
| QSA63 | | | | (90/10) | 9.9 | SIF | 6.2 | <0.1 | <0.1 | <0.1 | 6.1 |
| QSA64 | | | | | 8.9 | SGF | 1.3 | 117.2 | 139.5 | 139.1 | 1.3 |
| QSA65 | ASD9 | | 50 | Tetrahydrofuran/ | 8.6 | Water | 5.7 | 0.3 | <0.1 | <0.1 | 4.9 |
| QSA66 | | | | Water (50/50) | 10.7 | SIF | 6.6 | <0.1 | <0.1 | <0.1 | 5.8 |
| QSA67 | | | | | 9.7 | SGF | 1.3 | 81.3 | 55.2 | 23.5 | 1.3 |
| QSA68 | ASD30 | HPMC | 10 | Tetrahydrofuran/ | 8.5 | Water | 5.7 | 5.7 | 6.6 | 7.4 | 5.8 |
| QSA69 | | | | Water (50/50) | 9.8 | SIF | 6.7 | 7.5 | 6.1 | 6.7 | 6.7 |
| QSA70 | | | | | 8.4 | SGF | 1.3 | 108.7 | 116.5 | 89.6 | 1.3 |
| QSA71 | ASD33 | | 25 | Tetrahydrofuran/ | 9.1 | Water | 6.1 | 5.2 | 6.9 | 6.8 | 6.1 |
| QSA72 | | | | Water (50/50) | 8.7 | SIF | 6.8 | 3.9 | 4.4 | 3.6 | 6.7 |
| QSA73 | | | | | 9.3 | SGF | 1.3 | 111.0 | 97.8 | 93.3 | 1.3 |
| QSA74 | ASD34 | | 50 | Acetone/Methanol | 5.6 | Water | 6.4 | 7.3 | 8.4 | 6.4 | 6.4 |
| QSA75 | | | | (80/20) | 5.5 | SIF | 6.8 | 3.3 | 6.6 | 5.1 | 6.7 |
| QSA76 | | | | | 6.0 | SGF | 1.3 | 209.2 | 180.2 | 160.5 | 1.3 |
| QSA77 | ASD39 | Methyl cellulose | 10 | Tetrahydrofuran/ | 7.8 | Water | 6.4 | 2.7 | 2.3 | 1.4 | 6.4 |
| QSA78 | | | | Water (50/50) | 9.5 | SIF | 6.8 | 0.8 | 1.3 | 0.5 | 6.7 |
| QSA79 | | | | | 10.4 | SGF | 1.3 | 25.6 | 48.5 | 53.3 | 1.3 |
| QSA80 | ASD41 | | 25 | Chloroform/Ethanol | 7.8 | Water | 6.4 | 2.8 | 0.5 | 0.8 | 6.5 |
| QSA81 | | | | (50/50) | 9.3 | SIF | 6.8 | 1.4 | 0.8 | 1.2 | 6.7 |
| QSA82 | | | | | 6.6 | SGF | 1.3 | 29.7 | 49.4 | 64.8 | 1.3 |
| QSA83 | ASD45 | | 50 | Tetrahydrofuran/ | 6.5 | Water | 5.9 | 1.3 | 3.5 | 3.3 | 6.5 |
| QSA84 | | | | Water (50/50) | 7.5 | SIF | 6.8 | 1.4 | 1.8 | 1.8 | 6.7 |
| QSA85 | | | | | 7.9 | SGF | 1.3 | 57.5 | 69.2 | 75.8 | 1.3 |
| QSA95 | ASD101 | — | — | Dichloromethane | 6.0 | Water | 5.6 | <0.1 | <0.1 | <0.1 | 6.1 |
| QSA96 | | | | | 7.1 | SIF | 6.8 | <0.1 | <0.1 | <0.1 | 6.7 |
| QSA97 | | | | | 7.4 | SGF | 1.3 | 12.1 | 11.6 | 11.2 | 1.3 |
| QSA98 | ASD102 | | — | Tetrahydrofuran/ | 8.0 | Water | 6.1 | <0.1 | <0.1 | <0.1 | 5.5 |
| QSA99 | | | | Water (50/50) | 8.8 | SIF | 6.8 | <0.1 | <0.1 | <0.1 | 6.8 |
| QSA100 | | | | | 9.3 | SGF | 1.3 | 20.8 | 13.0 | 10.9 | 1.3 |
| QSA101 | ASD105 | | — | Acetone/Ethanol | 7.2 | Water | 6.3 | <0.1 | <0.1 | <0.1 | 6.0 |
| QSA102 | | | | (80/20) | 6.8 | SIF | 6.8 | <0.1 | <0.1 | <0.1 | 6.8 |
| QSA103 | | | | | 8.2 | SGF | 1.3 | 29.8 | 18.5 | 17.2 | 1.3 |

Solubility in Water

The solubility of crystalline Compound 1A in water was around 1 μg/mL. Starting the solubility determination with the amorphous Compound 1A, the solubility was below the detection limit of the HPLC method used. The dispersions prepared with Carbopol® 980 NF did not perform any better than crystalline Compound 1A and similar solubility values were determined. Dispersions prepared with methyl cellulose were a little better. Best performing were the dispersions prepared with HPMC. The latter dispersion resulted in concentrations of Compound 1A in solution of around 6 μg/mL. The dissolution was almost completed after 30 minutes and after 2 hours the concentration of Compound 1A had not changed much.

Although the improvement in solubility between crystalline Compound 1A and a dispersion in HPMC is about 6-fold it should be noted that only about 2% of Compound 1A present in the dispersion in fact went into solution.

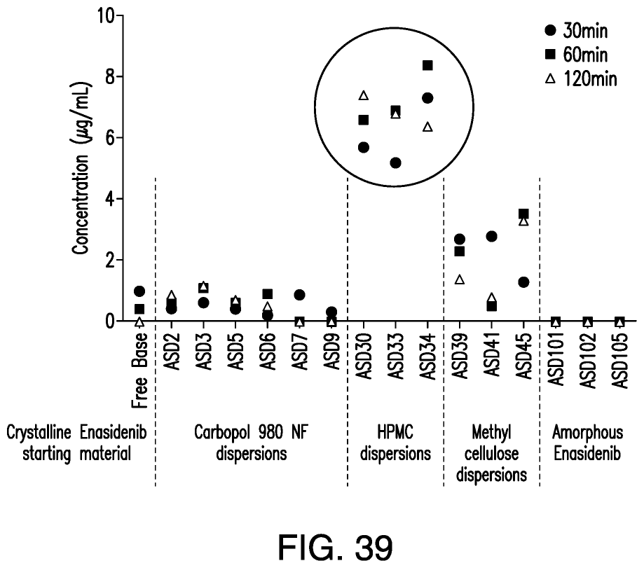

FIG. 39 provides a graphical representation of the solubility results of Compound 1A solid dispersions in water at 37° C. Best solubility results in water were determined for Compound 1A amorphous dispersions with HPMC. With 10% drug loading (ASD30), the solubility was increasing up to 2 hours. The maximum solubility was reached for the 10% drug load dispersion after 120 min. It seems that super saturation was not reached for the 10% drug load dispersion (ASD30) while with the 25% (ASD33) and 50% (ASD34) drug loadings the “parachute” effect was detected (solubility decrease).

Solubility in SIF

The solubility of Compound 1A in SIF was <0.1m/mL. The results obtained in the time resolved solubility study were very similar to those obtained in water. Again, the HPMC dispersion performed best, but still only a small amount of Compound 1A (±2%) was found back in solution.

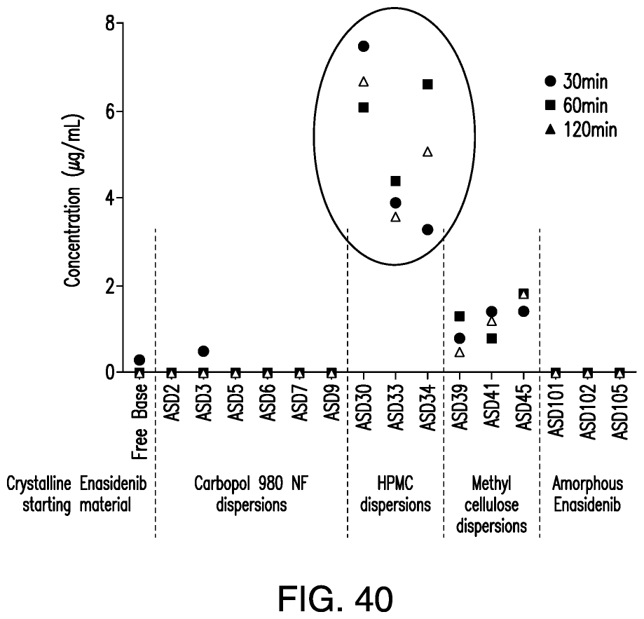

FIG. 40 provides a graphical representation of the solubility results of Compound 1A solid dispersions in SIF at 37° C. Best solubility results in SIF were determined for Compound 1A amorphous dispersions with HPMC. The maximum solubility was reached after 30 min for the 10% drug load (ASD30) and after 60 min for the 25% (ASD33) and 50% drug load (ASD34).

Solubility in SGF

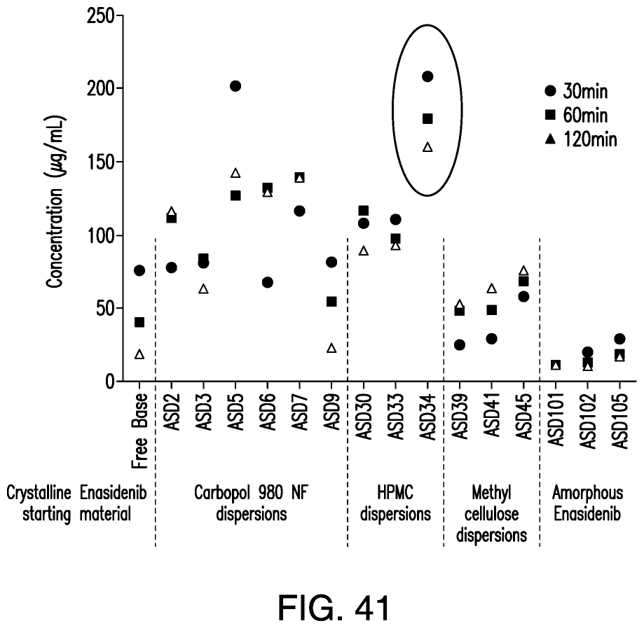

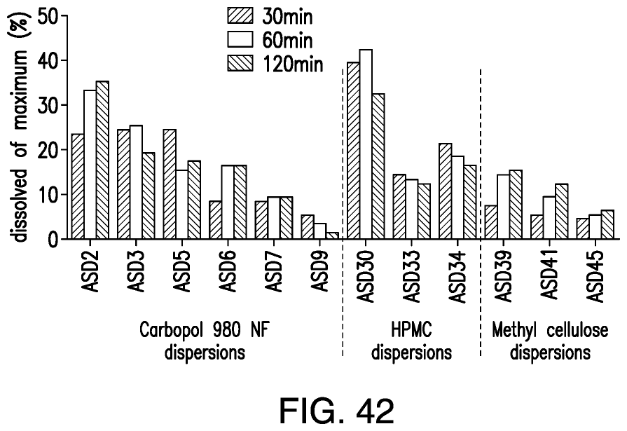

Crystalline Compound 1A reached concentrations in solution close to 80 μg/mL after 30 minutes which dropped to about 20 μg/mL after 2 hours (FIG. 41). In SGF all dispersions resulted in a supersaturated concentration of Compound 1A in solution. When the dissolution was rapid, typically the concentration of Compound 1A in solution dropped after 30 minutes. But when the dissolution rate was lower, the concentration of Compound 1A could reach a maximum after 2 hours. The absolute solubility values of the time resolved solubility determination experiments are presented in FIG. 41. FIG. 42 provides the relative solubility presented as function of the maximum achievable solubility when all Compound 1A present in the dispersion had gone into solution. This percentage depends on the drug load in the dispersion and the capability of the polymer to keep the drug from precipitating.

Overall, the ASDs prepared with different excipients and with different drug loads all gave improved solubility of Compound 1A in SGF (37° C.). In absolute sense, the dispersion prepared in HPMC with a drug load of 50% provided the highest solubility. Even after 2 hours the solubility still exceeded 150 μg/mL. From HPMC dispersions, between 20 to 40% of Compound 1A was released from the dispersion.

The dispersions prepared in Carbopol® resulted in solutions of Compound 1A with about 100 μg/mL concentrations. Both rapid as well as slow dissolutions were observed together with more fast precipitation and slow precipitation. The solvent system in which the dispersions were prepared seemed not to influence the performance except the 50% drug load dispersion prepared in THF/water (ASD9). This dispersion performed similar to crystalline Compound 1A. From Carbopol®, about 10 to 30% of Compound 1A released from the dispersions was found back in solution.

The dispersions prepared in methyl cellulose were performing poor. Around 10% of the drug released from the dispersions was found back in solution and the solubility was comparable to that obtained with crystalline Compound 1A.

### Example 6: Compound 1B—Starting Material Characterization

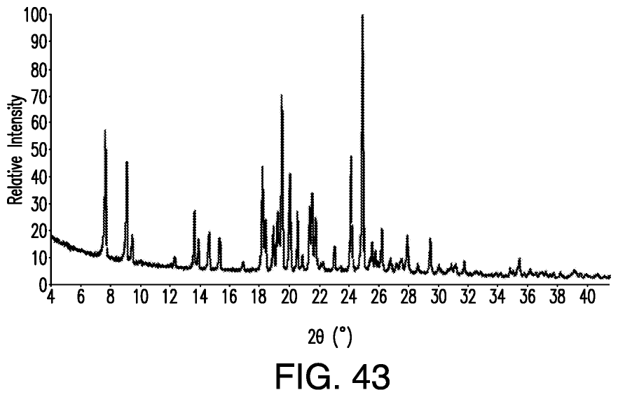

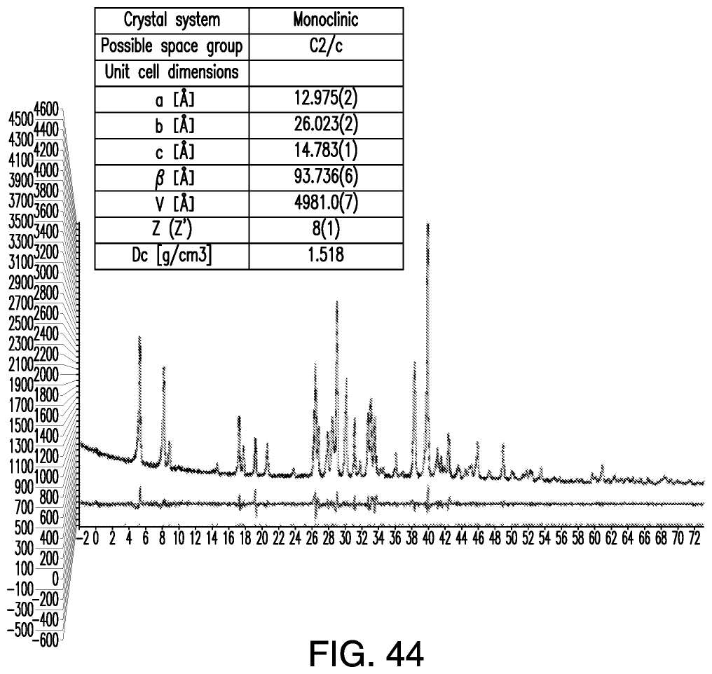

Compound 1B used in the following examples was a crystalline solid as confirmed by the High Resolution XRPD analysis, as provided in FIG. 43 This material was Form 3 of Compound 1B described in U.S. Pat. No. 9,738,625. For indexing of the HR-XRPD diffractogram the space group C2/c was chosen and refinement of cell parameters suggested that the material crystallized in a monoclinic crystal system (FIG. 44). FIG. 44 provides a graphical representation of the Whole Powder Pattern Decomposition (Pawley) analysis for Compound 1B powder pattern. In FIG. 44, the obtained powder pattern, the calculated powder pattern and the the difference between them are depicted. The calculated peak positions of the fitted cell are provided at the bottom. In the table on the right in FIG. 44, the suggested crystal system and cell parameters are summarized.

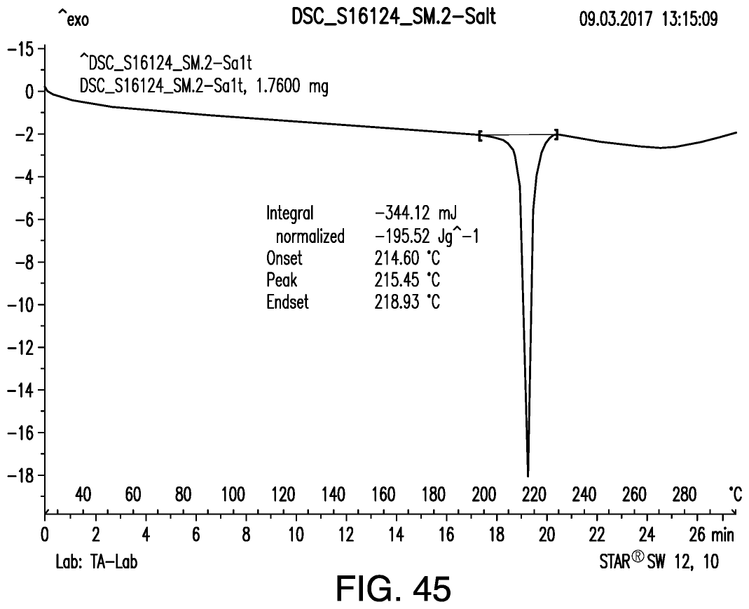

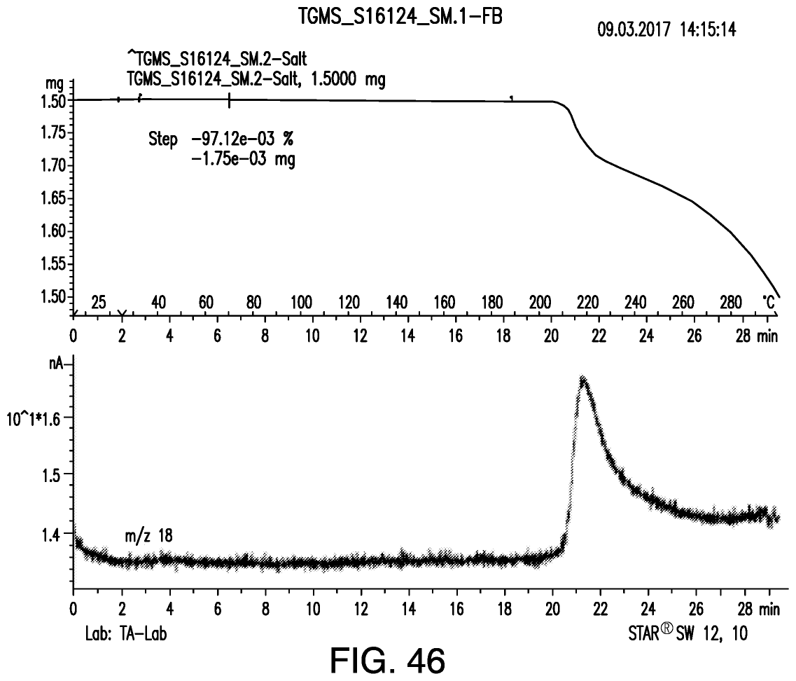

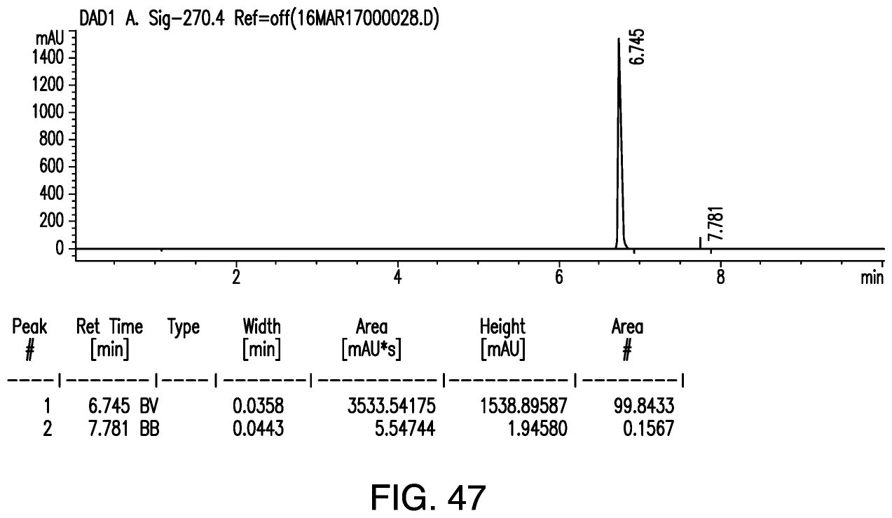

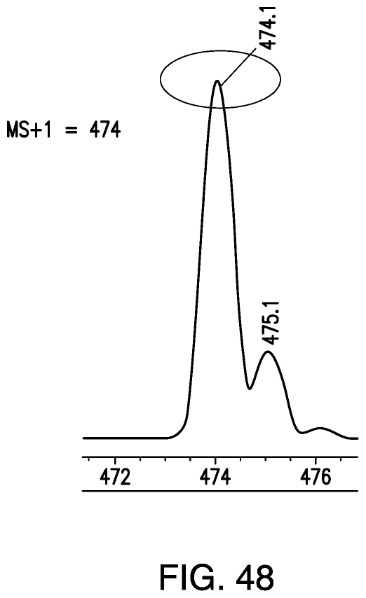

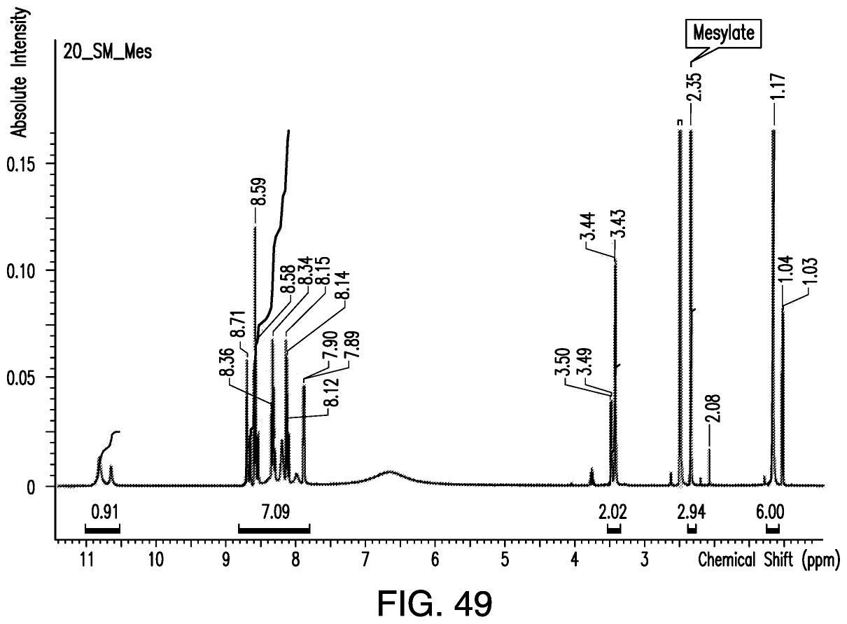

For reference purposes the material was characterized by thermal analysis, LCMS and proton NMR. The DSC thermogram indicated a melting point of 216.5° C. (FIG. 45). Thermal decomposition started immediately after melting and no significant mass loss was observed prior to melting/decomposition (FIG. 46). The LCMS analysis confirmed that the material was chemically pure and the m/z value was 473 g/mol, corresponding to the mass of Enasidenib (FIGS. 47 and 48). The 1H-NMR for the starting material is shown in FIG. 49.

The specifications for the polymers used in this study are presented in Table 1 above.

### Example 7: Solubility of Compound 1B

A quantitative (24-hour shake flask) thermodynamic solubility determination was performed on Compound 1B to aid in the selection of the screening solvents.

The solubility of Compound 1B was determined in 10 solvents at room temperature. Approximately 30-50 mg of Compound 1B was weighed in a standard 1.8 mL HPLC vial, subsequently a volume of solvent was added and the vials were left to equilibrate at room temperature with continuous stirring (see Table 12 for details). After 24 hours the solids were separated from the liquid by centrifugation and the liquid phase was further filtrated through a 0.2 μm PTFE filter to remove any particulate matter. The concentration of Compound 1A in solution was determined by HPLC-DAD analysis.

The calibration curve was made from two independent stock solutions of Compound 1A prepared in acetonitrile. The molecular masses of Compound 1A and 1B were taken into account to correct for the actual concentration in mg/mL.

The experimental conditions are presented in Table 12.

TABLE 12
Experimental conditions and results for the quantitative
solubility determination performed on Compound 1A:

| | Mass | | Volume | Solubility | | After |
|---|---|---|---|---|---|---|
| Exp ID | (mg) | Solvent | (μL) | (mg/mL) | Form | AAC |
| QSA30 | 51.0 | Tetrahydrofuran | 200 | 1.8 | Form 3 | Form 3 |
| QSA31 | 27.0 | Acetone | 200 | 0.9 | Form 3 | Form 3 |
| QSA32 | 30.2 | Acetonitrile | 200 | 0.5 | Form 3 | Form 3 |
| QSA33 | 31.8 | Water | 1000 | <0.1 | Form 2 | Form 2 |
| QSA34 | 47.1 | Methanol | 200 | 35.6 | Form 14 | Form M5 |
| QSA35 | 43.0 | Ethanol | 200 | 7.3 | Form 15 | Form M5 |
| QSA36 | 38.8 | 2-Propanol | 200 | 1.4 | Form 3 | Form 3 |
| QSA37 | 39.6 | Ethyl acetate | 200 | 0.2 | Form 3 | Form 3 |
| QSA38 | 30.3 | 1,4-Dioxane | 200 | 0.4 | Form 3 | Form 3 |
| QSA39 | 36.1 | Chloroform | 200 | 0.2 | Form 3 | Form 3 |

The residual solids were analyzed by HT-XRPD. The solids collected from the experiments performed in poorly soluble solvents did not showed solid form conversion. In all these experiments, Form 3 identical to the starting material was obtained, except in water where a hydrated form of the free base was identified (Form 2) suggesting that the salt disproportionate. From methanol and ethanol conversion to Form 14 and Form 15, respectively was observed. After exposure to accelerated aging conditions (40° C./70% RH) for two days, these forms converted to Form M5, which is a mixture of Form 14, Form 15 and Form 3 of Compound 1B. FIG. 74 provides an XRPD pattern for solid Form M5. Analysis of Form M5 by TGMS suggested that this form contained water or solvent.

Compound 1B showed a significantly lower solubility in the solvents tested compared to Compound 1A. In most of the solvents, Compound 1B was only very slightly or poorly soluble. Only in methanol, Compound 1B was soluble with about 35 mg/mL. Compound 1B was also practically insoluble in water.

The results of the solubility determination are summarized in Table 13.

TABLE 13

| Solvent | Solubility (mg/mL) | Form | After AAC |
|---|---|---|---|
| Methanol | 35.6 | Form 14 | Form M5 |
| Ethanol | 7.3 | Form 15 | Form M5 |
| Tetrahydrofuran | 1.8 | Form 3 | Form 3 |
| 2-Propanol | 1.4 | Form 3 | Form 3 |
| Acetone | 0.9 | Form 3 | Form 3 |
| Acetonitrile | 0.5 | Form 3 | Form 3 |
| 1,4-Dioxane | 0.4 | Form 3 | Form 3 |
| Chloroform | 0.2 | Form 3 | Form 3 |
| Ethyl acetate | 0.2 | Form 3 | Form 3 |
| Water | <0.1 | Form 2 | Form 2 |

### Example 8: Compound 1B—Anti-Precipitant Screening by the Solvent Shift Method

The anti-precipitation screen by the solvent shift method was performed with several excipients. This study was used for finding polymers that can maintain the supersaturated state of Compound 1B in solution. Based on the results of this screen, excipients were selected for the development of Compound 1B.

For the solvent shift method, a small aliquot of Compound 1B in DMSO solution was added to a polymer solution in SIF or SGF.

The excipients investigated in this study are presented in Table 4 above.

Prior to the solvent shift experiments, the kinetic solubility of Compound 1B was tested in SIF and SGF in order to choose the Compound 1B concentration suitable for the solvent shift experiments. Four different Compound 1B solutions (in DMSO) were prepared with concentrations of 60, 30, 15 and 5 mg/mL. Subsequently, 80 μL of these solutions were added to 4 mL of SGF and to 4 mL of SIF. In SGF precipitation was observed when using the solution with the highest concentration (60 mg/mL). The other three solutions did not precipitate indicating that in order to see an effect on the anti-precipitant properties of the polymers the initial Compound 1B concentration in DMSO has to be at least 60 mg/mL. In case of SIF, Compound 1B precipitated using all four concentrations. For the solvent shift experiments in SIF, an Compound 1B concentration in DMSO of 5 mg/mL was used.

The polymer solutions were prepared by weighing 0.6 mg of polymer in an 8 mL glass vial. The polymer was dissolved in 4 mL of SIF or SGF (polymer concentration of 0.15 mg/mL). To the polymer solution, 80 μL of Compound 1B solution in DMSO was added (5 mg/mL for the SIF experiments and 60 mg/mL for the SGF experiments, see Table 14). Table 14. Details of Compound 1B solutions in DMSO. The ratio of Compound 1B:Polymer is based on the ratio of 80 μL Compound 1B solution in 4 mL polymer solution of 0.15 mg/mL.

TABLE 14

| | | | Concentration | Concentration | |
|---|---|---|---|---|---|
| Stock | | | of API | determined | Ratio in |
| for | API | DMSO | in DMSO | by HPLC | experiment |
| Medium | Mg | mL | mg/mL | mg/mL | API:Polymer |
| SIF | 10.2 | 2.00 | 5.1 | 4.4 | 2:3 |
| SGF | 89.5 | 1.50 | 59.6 | 58.0 | 8:1 |

The vials were left to equilibrate at 25° C. with continuous stirring. As a reference, two control experiments were performed. In control experiment 1, crystalline Compound 1B was added to a pre-mixed solution of medium containing 2% DMSO. In control experiment 2, 80 μL of Compound 1B in DMSO was added to the medium without polymer in the same manner as the experiments.

After 0.5, 1, 2, 4 and 24 hours a small aliquot of mother liquor was taken and filtrated through a 0.45 μm PTFE filter to remove any particulate matter. The filtrate was diluted in acetonitrile, to prevent the sample from precipitation. The concentration of Compound 1A in solution was determined by HPLC analysis. The calibration curve was made from two independent stock solutions of Compound 1A prepared in acetonitrile. The molecular masses of Compound 1A and 1B were taken into account to correct for the actual concentration in mg/mL.

The experimental details and results of the experiments in SIF are described in Table 15 and of the experiments in SGF in Table 16. The concentration of Compound 1B in solution was determined by HPLC analysis after 0.5, 1, 2, 4 and 24 hours.

TABLE 15
Experimental conditions and results of the solvent shift precipitation experiments in SIF.

| | | Concentration (mg/mL) | | | | |
|---|---|---|---|---|---|---|
| Exp ID | Polymer | 0.5 h | 1 h | 2 h | 4 h | 24 h |
| REF201 | Hydroxyethyl cellulose (HEC) | 0.003 | <0.001 | <0.001 | <0.001 | <0.001 |
| REF202 | Hydroxypropyl methyl cellulose (HPMC) | 0.021 | 0.023 | 0.024 | 0.013 | <0.001 |
| REF203 | Hydroxypropyl cellulose (HPC) | 0.001 | 0.017 | 0.016 | <0.001 | <0.001 |
| REF204 | Eudragit L100 | 0.001 | 0.008 | 0.000 | <0.001 | <0.001 |
| REF205 | Polyethylene Glycol 6000 (PEG 6000) | <0.001 | <0.001 | <0.001 | <0.001 | <0.001 |
| REF206 | Polyvinylpyrrolidone K30 (PVP K30) | 0.004 | 0.014 | 0.004 | <0.001 | <0.001 |
| REF207 | Poly(1-vinylpyrrolidone-co-vinyl | 0.004 | 0.007 | 0.003 | <0.001 | <0.001 |
| | acetate) (PVP-VA) | | | | | |
| REF208 | Pluronic F-68 | <0.001 | <0.001 | <0.001 | <0.001 | <0.001 |
| REF209 | D-α-Tocopherol polyethylene | <0.001 | <0.001 | <0.001 | <0.001 | <0.001 |
| | glycol 1000 succinate | | | | | |
| REF210 | Carbopol ® 980 NF Polymer | <0.001 | 0.014 | 0.017 | 0.025 | 0.015 |
| REF211 | Soluplus ® | 0.019 | 0.024 | 0.017 | 0.013 | 0.020 |
| REF212 | Povacoat Type MP | 0.023 | 0.013 | <0.001 | <0.001 | <0.001 |
| REF213 | Kollicoat IR | 0.012 | <0.001 | <0.001 | <0.001 | <0.001 |
| REF214 | Sureteric | 0.001 | <0.001 | <0.001 | <0.001 | <0.001 |
| REF215 | API crystalline solid | 0.000 | 0.000 | <0.001 | <0.001 | <0.001 |
| REF216 | API in DMSO | 0.006 | 0.010 | 0.005 | <0.001 | <0.001 |

TABLE 16
Experimental conditions and results of the solvent
shift precipitation experiments in SGF.

| | | Concentration (mg/mL) | | | | |
|---|---|---|---|---|---|---|
| Exp ID | Polymer | 0.5 h | 1 h | 2 h | 4 h | 24 h |
| REF217 | Hydroxyethyl cellulose (HEC) | 0.02 | 0.01 | <0.01 | 0.01 | <0.01 |
| REF218 | Hydroxypropyl methyl cellulose (HPMC) | 0.57 | 0.37 | 0.30 | 0.18 | 0.07 |
| REF219 | Hydroxypropyl cellulose (HPC) | 0.23 | 0.15 | 0.10 | 0.07 | 0.04 |
| REF220 | Eudragit E100 | 0.92 | 0.67 | 0.14 | 0.10 | 0.04 |
| REF221 | Polyethylene Glycol 6000 (PEG 6000) | 0.01 | 0.01 | 0.00 | 0.01 | <0.01 |
| REF222 | Polyvinylpyrrolidone K30 (PVP K30) | 0.28 | 0.09 | 0.04 | 0.03 | <0.01 |
| REF223 | Poly(1-vinylpyrrolidone-co-vinyl | 0.15 | 0.07 | 0.04 | 0.03 | <0.01 |
| | acetate) (PVP-VA) | | | | | |
| REF224 | Pluronic F-68 | 0.04 | 0.03 | 0.01 | 0.01 | <0.01 |
| REF225 | D-α-Tocopherol polyethylene | 0.05 | 0.04 | 0.03 | 0.04 | 0.01 |
| | glycol 1000 succinate | | | | | |
| REF226 | Carbopol ® 980 NF Polymer | 0.27 | 0.22 | 0.23 | 0.16 | <0.01 |
| REF227 | Soluplus ® | 0.81 | 0.57 | 0.53 | 0.56 | 0.12 |
| REF228 | Povacoat Type MP | 0.08 | 0.06 | 0.05 | 0.04 | 0.02 |
| REF229 | Kollicoat IR | 0.11 | 0.06 | 0.03 | 0.03 | 0.01 |
| REF230 | Methyl cellulose | 0.46 | 0.32 | 0.21 | 0.13 | 0.04 |
| REF231 | API crystalline solid | <0.01 | <0.01 | <0.01 | <0.01 | <0.01 |
| REF232 | API in DMSO | 0.02 | 0.00 | <0.01 | <0.01 | <0.01 |

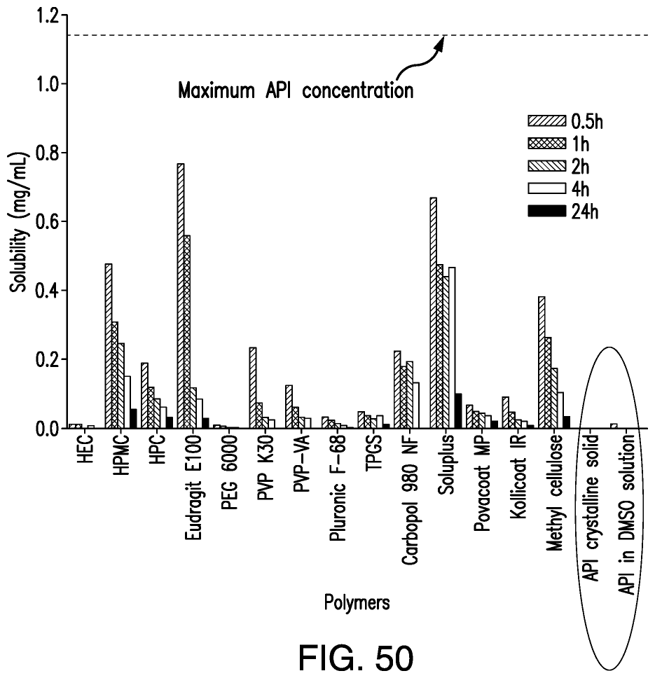

The results of the experiments performed in SGF polymer solutions are summarized in FIG. 50. The control experiments are highlighted with oval. Compared to the control experiments, a significant solubility improvement was observed with most of the polymers. Higher super saturation and best anti-precipitant properties were observed with Soluplus®. A part of the Compound 1B precipitated quickly; however, the Compound 1B concentration was maintained at 0.5 mg/mL for 4 hours. Other promising polymers were Carbopol®, HPMC, Eudragit E100 and methyl cellulose.

Using SGF HPMC, Eudragit E100 and methyl cellulose solutions, Compound 1B precipitated quite drastically during the 24 hours test period. With Carbopol® a limited supersaturation at 0.2 mg/mL was observed which was maintained over 24 hours. Based on these results it is assumed that Compound 1B has an affinity with these five polymers and therefore, they are promising polymers for further development of an amorphous dispersion.

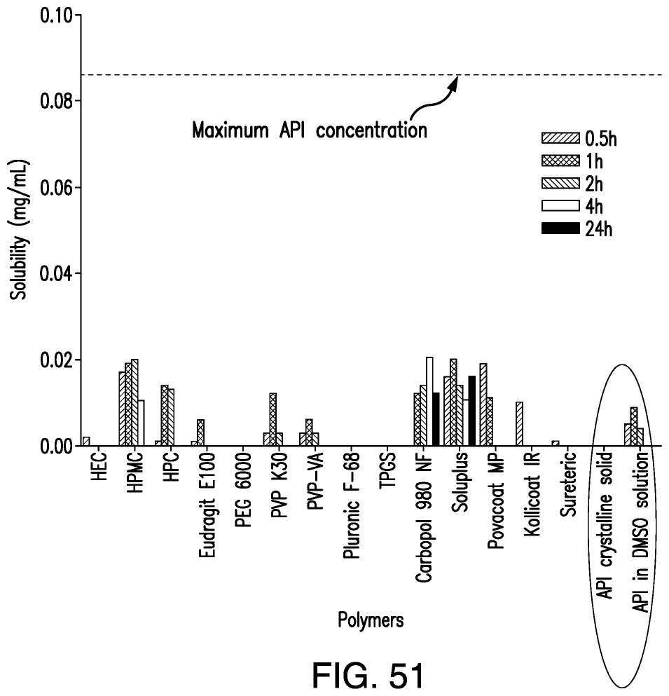

The results of the experiments performed in SIF are summarized in FIG. 51. Crystalline Compound 1B was practically insoluble in SIF as no Compound 1B was detected in solution. From the control experiment performed with Compound 1B in solution, very low solubility values could be detected for at least 2 hours (<0.01 mg/mL). Due to the low solubility determined in the SIF solutions, the polymers for the preparation of the amorphous solid dispersions were chosen based on the SGF results.

### Example 9: Amorphous Solid Dispersion Screen for Compound 1B

The amorphous solid dispersion screen employed five polymers: Soluplus®, Carbopol®, Eudragit E100, HPMC and methyl cellulose. Three different solvent systems and three drug loads were used per polymer. For the preparation of the dispersions, solid Compound 1B and polymer were weighed in 8 mL glass vials. Solvent was added until a homogenous solution was obtained. The solutions were frozen in liquid nitrogen followed by drying using Crys Alpha 2-4 LO freeze-drier for 18 hours. Control experiments were performed with Compound 1B in different solvents (without excipients).

The amorphous dispersions were all harvested and analyzed by HT-XRPD. Subsequently, all the solids were exposed to 4 weeks of accelerated aging conditions (AAC) at 40° C. and at 70% RH. A digital image was taken directly prior to the HT-XRPD measurement. The experimental conditions and results are reported in Table 17. The solids were analyzed by HT-XRPD. All samples were re-analyzed after exposure to AAC for 4 weeks (AAC). In Table 17, “Am” means amorphous and “Am glassy” means that films or glassy materials were identified. After the stability study in most of the cases, the solids became deliquescent (oily/liquid). Blank experiments were taken along for reference purposes.

TABLE 17
Experimental conditions and results of the amorphous solid dispersion experiments.

| | Mass | | Mass | Drug | | | | |
|---|---|---|---|---|---|---|---|---|
| | Compound 1B | | Polymer | load | | Volume | XRPD | |
| Exp ID | (mg) | Polymer | (mg) | (%) | Solvent | (mL) | Form | AAC |
| ASD46 | 5.3 | Carbopol ® 980 | 46.4 | 10 | 2,2,2-Trifluoroethanol/ | 6.60 | Am | Crystalline |
| | | NF Polymer | | | Water (80/20) | | | |
| ASD47 | 5.3 | Carbopol ® 980 | 47.7 | 10 | Tetrahydrofuran/ | 3.00 | Am | Am |
| | | NF Polymer | | | Water (50/50) | | | |
| ASD48 | 5.2 | Carbopol ® 980 | 46.8 | 10 | Methanol/Water | 5.20 | Am | Deliquescent |
| | | NF Polymer | | | (50/50) | | | |
| ASD49 | 12.7 | Carbopol ® 980 | 37.8 | 25 | 2,2,2-Trifluoroethanol/ | 6.10 | Am | Deliquescent |
| | | NF Polymer | | | Water (80/20) | | | |
| ASD50 | 12.6 | Carbopol ® 980 | 37.8 | 25 | Tetrahydrofuran/ | 4.40 | Crystalline | Deliquescent |
| | | NF Polymer | | | Water (50/50) | | | |
| ASD51 | 12.7 | Carbopol ® 980 | 38.1 | 25 | Methanol/Water | 5.20 | Am | Deliquescent |
| | | NF Polymer | | | (50/50) | | | |
| ASD52 | 25.1 | Carbopol ® 980 | 25.1 | 50 | 2,2,2-Trifluoroethanol/ | 6.10 | Crystalline | Crystalline |
| | | NF Polymer | | | Water (80/20) | | | |
| ASD53 | 25.1 | Carbopol ® 980 | 25.1 | 50 | Tetrahydrofuran/ | 4.50 | Am | Crystalline |
| | | NF Polymer | | | Water (50/50) | | | |
| ASD54 | 25.0 | Carbopol ® 980 | 25.0 | 50 | Methanol/Water | 5.40 | Am | Crystalline |
| | | NF Polymer | | | (50/50) | | | |
| ASD55 | 5.1 | Soluplus ® | 45.9 | 10 | 2,2,2-Trifluoroethanol/ | 1.05 | Am | Deliquescent |
| | | | | | Water (80/20) | | | |
| ASD56 | 5.3 | Soluplus ® | 47.7 | 10 | Chloroform/Ethanol | 0.60 | Am | Deliquescent |
| | | | | | (50/50) | | | |
| ASD57 | 5.1 | Soluplus ® | 45.9 | 10 | Tetrahydrofuran/ | 1.40 | Crystalline | Deliquescent |
| | | | | | Water (50/50) | | | |
| ASD58 | 12.5 | Soluplus ® | 37.5 | 25 | 2,2,2-Trifluoroethanol/ | 1.10 | Am | Deliquescent |
| | | | | | Water (80/20) | | | |
| ASD59 | 12.7 | Soluplus ® | 38.1 | 25 | Chloroform/Ethanol | 2.80 | Crystalline | Crystalline |
| | | | | | (50/50) | | | |
| ASD60 | 12.4 | Soluplus ® | 37.2 | 25 | Tetrahydrofuran/ | 1.30 | Am | Crystalline |
| | | | | | Water (50/50) | | | |
| ASD61 | 25.2 | Soluplus ® | 25.2 | 50 | 2,2,2-Trifluoroethanol/ | 1.10 | Crystalline | Crystalline |
| | | | | | Water (80/20) | | | |
| ASD62 | 25.0 | Soluplus ® | 25.0 | 50 | Chloroform/Ethanol | 4.00 | Crystalline | Crystalline |
| | | | | | (50/50) | | | |
| ASD63 | 25.2 | Soluplus ® | 25.2 | 50 | Tetrahydrofuran/ | 1.50 | Am | Crystalline |
| | | | | | Water (50/50) | | | |
| ASD64 | 5.1 | Eudragit E100 | 45.9 | 10 | Methanol/Water | 1.00 | Am glassy | Deliquescent |
| | | | | | (80/20) | | | |
| ASD65 | 5.0 | Eudragit E100 | 45.0 | 10 | Dichloromethane/ | 0.50 | Am glassy | Deliquescent |
| | | | | | Methanol (50/50) | | | |
| ASD66 | 5.4 | Eudragit E100 | 48.6 | 10 | Tetrahydrofuran/ | 2.40 | Am | Deliquescent |
| | | | | | Water (50/50) | | | |
| ASD67 | 12.6 | Eudragit E100 | 37.8 | 25 | Methanol/Water | 1.00 | Am | Deliquescent |
| | | | | | (80/20) | | | |
| ASD68 | 12.6 | Eudragit E100 | 37.8 | 25 | Dichloromethane/ | 0.60 | Crystalline | Crystalline |
| | | | | | Methanol (50/50) | | | |
| ASD69 | 12.5 | Eudragit E100 | 37.5 | 25 | Tetrahydrofuran/ | 0.80 | Am | Deliquescent |
| | | | | | Water (50/50) | | | |
| ASD70 | 25.3 | Eudragit E100 | 25.3 | 50 | Methanol/Water | 1.00 | Crystalline | Crystalline |
| | | | | | (80/20) | | | |
| ASD71 | 25.2 | Eudragit E100 | 25.2 | 50 | Dichloromethane/ | 0.80 | Crystalline | Crystalline |
| | | | | | Methanol (50/50) | | | |
| ASD72 | 25.1 | Eudragit E100 | 25.1 | 50 | Tetrahydrofuran/ | 0.90 | Am | Deliquescent |
| | | | | | Water (50/50) | | | |
| ASD73 | 5.0 | Hydroxypropyl | 45.0 | 10 | 2,2,2-Trifluoroethanol/ | 1.10 | Am | Deliquescent |
| | | methyl cellulose | | | Water (80/20) | | | |
| ASD74 | 5.1 | Hydroxypropyl | 45.9 | 10 | Dichloromethane/ | 4.10 | Am glassy | Deliquescent |
| | | methyl cellulose | | | Methanol (50/50) | | | |
| ASD75 | 4.9 | Hydroxypropyl | 44.1 | 10 | Acetone/Methanol | 2.10 | Am glassy | Deliquescent |
| | | methyl cellulose | | | (50/50) | | | |
| ASD76 | 12.9 | Hydroxypropyl | 38.7 | 25 | 2,2,2-Trifluoroethanol/ | 1.10 | Am | Am |
| | | methyl cellulose | | | Water (80/20) | | | |
| ASD77 | 12.8 | Hydroxypropyl | 38.4 | 25 | Dichloromethane/ | 2.40 | Am glassy | Deliquescent |
| | | methyl cellulose | | | Methanol (50/50) | | | |
| ASD78 | 12.4 | Hydroxypropyl | 37.2 | 25 | Acetone/Methanol | 2.20 | Am glassy | Deliquescent |
| | | methyl cellulose | | | (50/50) | | | |
| ASD79 | 25.2 | Hydroxypropyl | 25.2 | 50 | 2,2,2-Trifluoroethanol/ | 1.10 | Am | Crystalline |
| | | methyl cellulose | | | Water (80/20) | | | |
| ASD80 | 25.0 | Hydroxypropyl | 25.0 | 50 | Dichloromethane/ | 2.20 | Am glassy | Crystalline |
| | | methyl cellulose | | | Methanol (50/50) | | | |
| ASD81 | 25.1 | Hydroxypropyl | 25.1 | 50 | Acetone/Methanol | 2.6 | Am glassy | Crystalline |
| | | methyl cellulose | | | (50/50) | | | |
| ASD82 | 5.0 | Methyl cellulose | 44.9 | 10 | Chloroform/Ethanol | 2.00 | Am | Am |
| | | | | | (50/50) | | | |
| ASD83 | 5.1 | Methyl cellulose | 45.8 | 10 | 2,2,2-Trifluoroethanol/ | 4.30 | Am | Crystalline |
| | | | | | Water (80/20) | | | |
| ASD84 | 5.1 | Methyl cellulose | 45.8 | 10 | Methanol/Water | 4.60 | Am | Deliquescent |
| | | | | | (50/50) | | | |
| ASD85 | 12.5 | Methyl cellulose | 37.4 | 25 | Chloroform/Ethanol | 2.00 | Am | Crystalline |
| | | | | | (50/50) | | | |
| ASD86 | 12.7 | Methyl cellulose | 38.0 | 25 | 2,2,2-Trifluoroethanol/ | 310 | Am | Crystalline |
| | | | | | Water (80/20) | | | |
| ASD87 | 12.7 | Methyl cellulose | 38.0 | 25 | Methanol/Water | 4.20 | Am | Crystalline |
| | | | | | (50/50) | | | |
| ASD88 | 25.4 | Methyl cellulose | 25.4 | 50 | Chloroform/Ethanol | 3.20 | Am | Crystalline |
| | | | | | (50/50) | | | |
| ASD89 | 24.9 | Methyl cellulose | 24.9 | 50 | 2,2,2-Trifluoroethanol/ | 3.10 | Am | Crystalline |
| | | | | | Water (80/20) | | | |
| ASD90 | 25.1 | Methyl cellulose | 25.1 | 50 | Methanol/Water | 4.40 | Am | Crystalline |
| | | | | | (50/50) | | | |
| ASD108 | 51.6 | — | — | 100 | Dichloromethane/ | 1.00 | Form 14 | Form 11 |
| | | | | | Methanol (50/50) | | | |
| ASD109 | 51.1 | — | — | 100 | Chloroform/Ethanol | 3.40 | Form 15 | Form 11 |
| | | | | | (50/50) | | | |
| ASD110 | 49.9 | — | — | 100 | Tetrahydrofuran/ | 0.60 | Am | Form 11 |
| | | | | | Water (50/50) | | | |
| ASD111 | 50.1 | — | — | 100 | 2,2,2-Trifluoroethanol/ | 1.00 | Low | Form 11 |
| | | | | | Water (80/20) | | crystalline | |
| ASD112 | 50.0 | — | — | 100 | Methanol/Water | 2.00 | M8 | Form 11 |
| | | | | | (50/50) | | | |
| ASD113 | 51.5 | — | — | 100 | Methanol/Water | 1.00 | M8 | Form 11 |
| | | | | | (80/20) | | | |
| ASD114 | 51.1 | — | — | 100 | Acetone/Methanol | 0.60 | Form 14 | Form 11 |
| | | | | | (50/50) | | | |

With all five polymers, amorphous solid dispersions were identified although most of the dispersions were highly hygroscopic and became deliquescent upon exposure to AAC (low drug load). With high drug loads, precipitation of Compound 1B in the dispersion was observed. Three solid dispersions were physically stable during exposure to AAC. Stable amorphous solid dispersions were obtained with Carbopol® (10% Compound 1B), HPMC (25% Compound 1B) and methyl cellulose (10% Compound 1B). With Soluplus® and Eudragit E100 no stable dispersions were found. The stable dispersions were further characterized by DSC and TGMS.

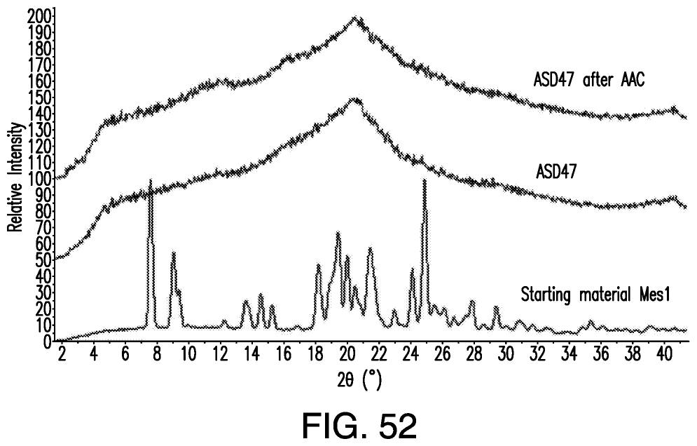

FIG. 52 provides an overlay of HT-XRPD patterns (from bottom to top): crystalline Compound 1B, amorphous solid dispersion prepared with Carbopol® 980 NF and 10% drug load in THF/water (50/50) (Exp. ID ASD47) before and after exposure to accelerated aging conditions for 4 weeks. The amorphous solid dispersion remained amorphous after the stability study.

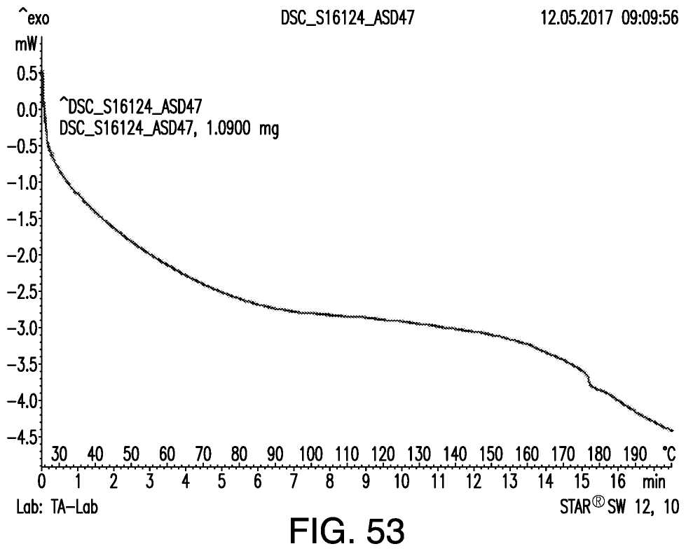

FIG. 53 provides a DSC spectrum (with a heating rate of 10° C./min and a pierced pan) of the amorphous solid dispersion prepared with Carbopol® 980 NF and 10% drug load in THF/water (50/50) (Exp. ID ASD47). No melting event was recorded, confirming the lack of crystalline material.

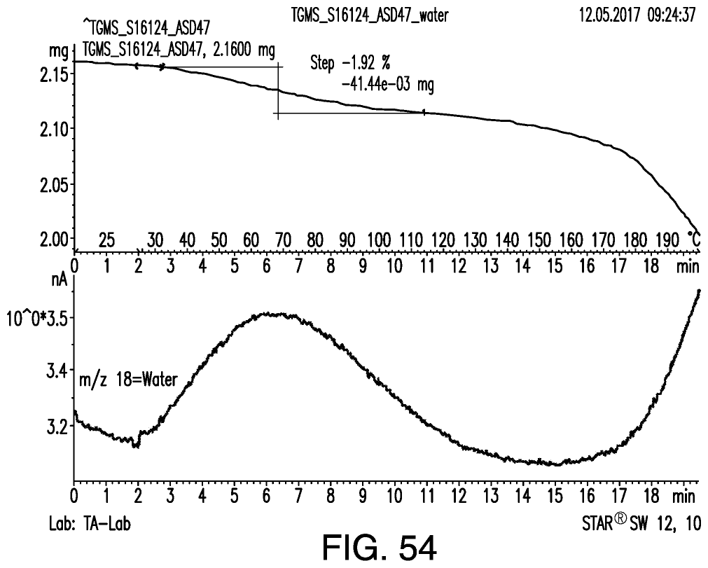

FIG. 54 provides a TGMS spectrum (with a heating rate of 10° C./min) of amorphous solid dispersion prepared with Carbopol® 980 NF and 10% drug load in THF/water (50/50) (Exp. ID ASD47). The TGMS signal shows a mass loss of 1.9% attributed to water. The thermal decomposition was observed after 180° C.

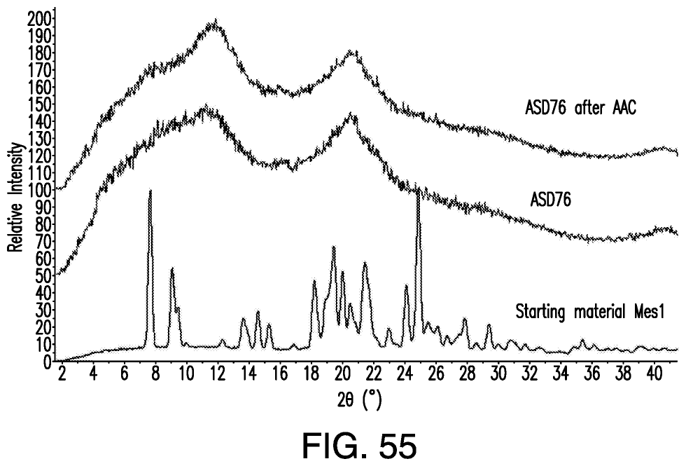

FIG. 55 provides an overlay of HTXRPD patterns (from bottom to top): crystalline Enasidenib mesylate, amorphous solid dispersion prepared with HPMC and 25% drug load in TFE/water (80/20) (Exp. ID ASD76) before and after exposure to accelerated aging conditions (40° C./70% RH) for 4 weeks. The solids remained amorphous.

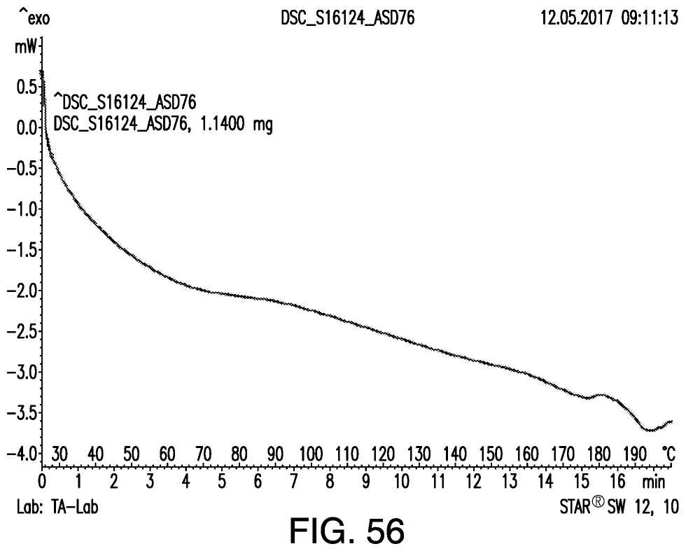

FIG. 56 provides a DSC spectrum (with a heating rate of 10° C./min and a pierced pan) of the amorphous solid dispersion prepared with HPMC and 25% drug load in TFE/water (80/20) (Exp. ID ASD76). No melting event was recorded, confirming the lack of crystalline material.

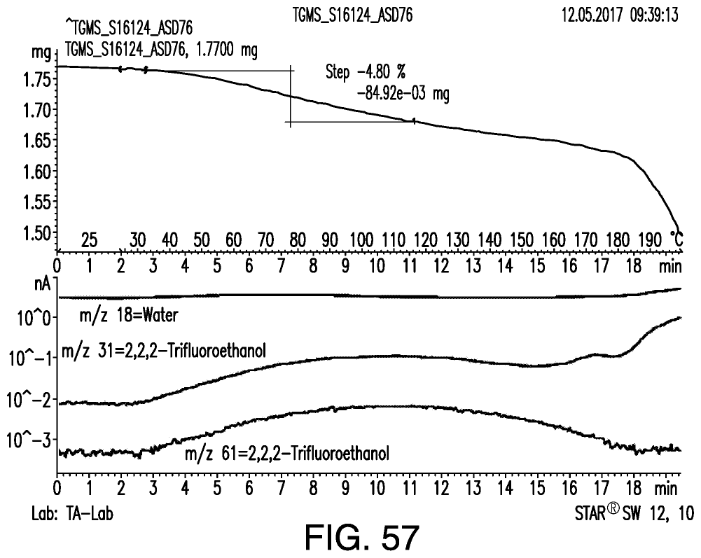

FIG. 57 provides a TGMS spectrum (with a heating rate of 10° C./min) of the amorphous solid dispersion prepared with HPMC and 25% drug load in TFE/water (80/20) (Exp. ID ASD76). The TGMS signal shows a mass loss of 4.8% attributed to TFE and water. The thermal decomposition was observed at 180° C.

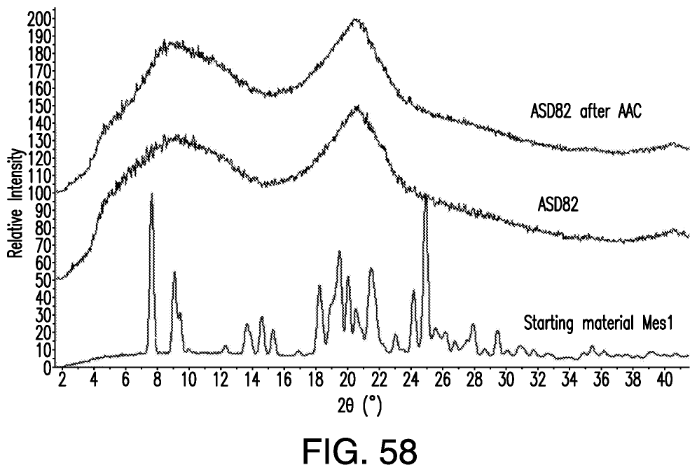

FIG. 58 provides an overlay of HT-XRPD patterns (from bottom to top): crystalline Enasidenib mesylate, amorphous solid dispersion prepared with methyl cellulose and 10% drug load in chloroform/ethanol (50/50) (Exp. ID ASD82) before and after exposure to accelerated aging conditions (40° C./70% RH) for 4 weeks. The solids remained amorphous.

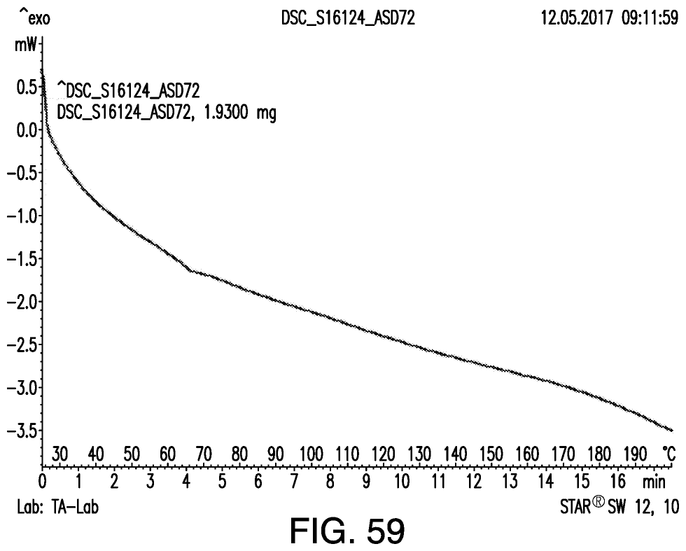

FIG. 59 provides a DSC spectrum (with a heating rate of 10° C./min and a pierced pan) of the amorphous solid dispersion obtained with 10% methyl cellulose in chloroform/ethanol (50/50) (Exp. ID ASD82). No melting event was recorded, confirming the lack of crystalline material.

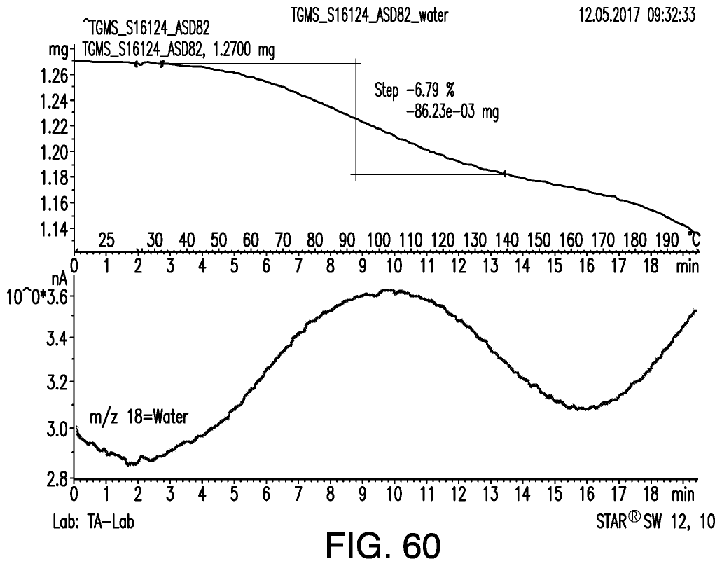

FIG. 60 provides a TGMS spectrum (with a heating rate of 10° C./min) of the amorphous solid dispersion obtained with 10% methyl cellulose in chloroform/ethanol (50/50) (Exp. ID ASD82). The TGMS signal shows a mass loss of 6.8% attributed to water. The thermal decomposition was observed at 180° C.

The DSC analysis confirmed the amorphous nature of Compound 1B in the dispersions and revealed no temperature induced crystallization. The amorphous solid dispersion prepared with Carbopol® showed the least residual water (about 2%) while the dispersion prepared with HPMC and methyl cellulose showed 5 and 7% of residual solvent and water, respectively. From these thermal analyses, it became also apparent that after the solvent loss Compound 1B did not crystallize. Therefore, it might be possible to reduce the solvent content by additional drying.

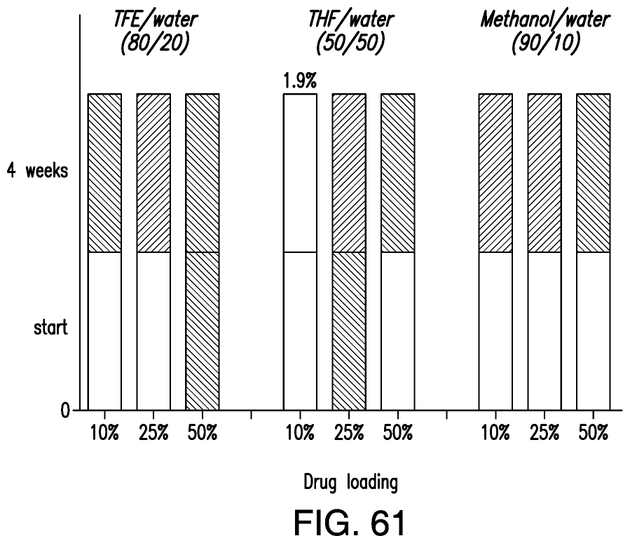

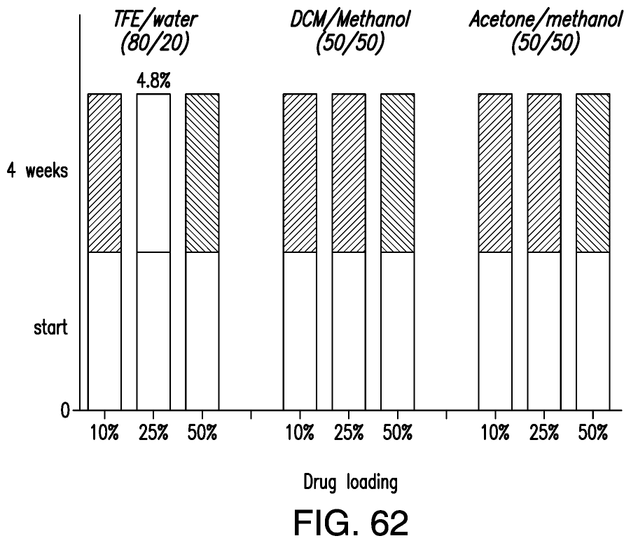

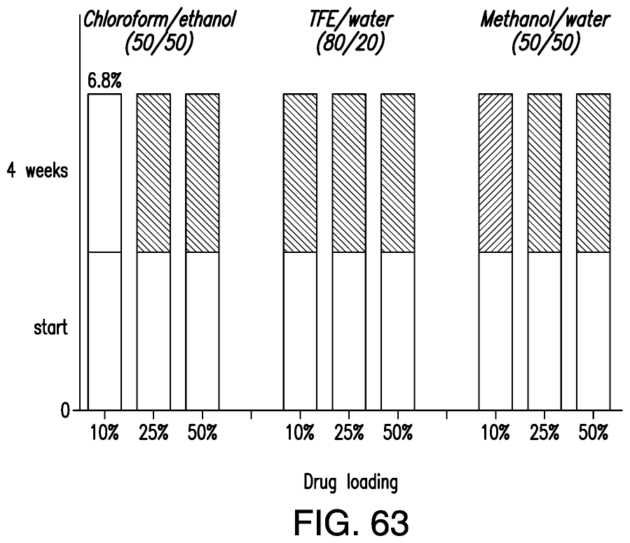

The graphical representation of the amorphous dispersion including the loss on drying determined by TGMS are presented in FIG. 61 for Carbopol®, FIG. 62 for HPMC, and FIG. 63 for methyl cellulose. In FIGS. 61-63, each bar represents a solid dispersion.

FIG. 61 provides a graphical representation of the solid dispersions prepared with Carbopol® 980 NF in three solvents systems before and after stability for 4 weeks. The dispersion with 10% drug load in THF/water (50/50) was amorphous powder. The dispersions with 25% drug load in TFE/water (80/20), and 10% and 25% drug load in methanol/water (90/10) were amorphous dispersions that became deliquescent upon exposure to AAC. The dispersions with 10% drug load in TFE/water (80/20), 50% drug load in THF/water (50/50), and 50% drug load in methanol/water (90/10) were amorphous dispersions that showed traces of the crystalline API in the XRPD diffractogram upon exposure to AAC. The dispersions with 50% drug load in TFE/water (80/20), showed traces of the crystalline API in the XRPD diffractogram prior to and upon exposure to AAC. The dispersions with 25% drug load in THF/water (50/50) showed traces of the crystalline API in the XRPD diffractogram and became deliquescent upon exposure to AAC. The loss on drying determined by TGMS analysis on the amorphous solid dispersions is presented above each bar.

FIG. 62 provides a graphical representation of the solid dispersions prepared with HPMC in three solvents systems before and after stability for 4 weeks. The dispersions with 25% drug load in TFE/water (80/20) was amorphous powder. The dispersions with 10% drug load in TFE/water (80/20), and 10% and 25% drug load in DCM/methanol (50/50), and and 10% and 25% drug load in acetone/methanol (50/50) were amorphous dispersions that became deliquescent upon exposure to AAC. The dispersions with 50% drug load in TFE/water (80/20), 50% drug load in DCM/methanol (50/50), and 50% drug load in acetone/methanol (50/50) were amorphous dispersions that showed traces of the crystalline API in the XRPD diffractogram upon exposure to AAC. The loss on drying determined by TGMS analysis on the amorphous solid dispersions is presented above each bar.

FIG. 63 provides a graphical representation of the solid dispersion prepared with methyl cellulose in three solvents systems before and after stability for 4 weeks. The dispersions with 10% drug load in chloroform/ethanol (50/50) was amorphous powder. The dispersions with 25% and 50% drug loads in chloroform/ethanol (50/50), 10%, 25% and 50% drug loads in TFE/water (80/20), 25% and 50% drug load in methanol/water (50/50) were amorphous dispersions that showed traces of the crystalline API in the XRPD diffractogram upon exposure to AAC. The dispersion with 10% drug loads in methanol/water (50/50) was amorphous dispersion that became deliquescent upon exposure to AAC. The loss on drying determined by TGMS analysis is presented above each bar.

### Example 9: Solubility Determination of Amorphous Solid Dispersions of Compound 1B

The thermodynamic solubility of the dispersions was determined in water, SIF and SGF at 37° C. SIF and SGF were prepared according to the description in the USP test solutions and were prepared without enzymes. The solubility of crystalline Compound 1B was determined as reference. Additionally the solubility of amorphous Compound 1B was determined.

For the solubility determination, the amorphous solid dispersions were weighed in 8 mL glass vials and 3 mL of the medium was added. The suspensions were incubated at 37° C. under continuous stirring. The solubility was determined after 0.5, 1 and 2 hours. At each equilibration time, an aliquot of the sample was filtered through a 0.45 μm PTFE filter to remove any particulate matters. The filtrate was diluted in acetonitrile and analyzed by HPLC to determine the concentration of solute. The pH was recorded at the start and at the end of the equilibration time. The experimental details and results are reported in Table 18.

TABLE 18

| | | | | | Mass | Mass | | | Concentration API | | | |
|---|---|---|---|---|---|---|---|---|---|---|---|---|
| | Solid | | Drug | Dispersion | dispersion | API | | pH | (μg/mL) | | | pH |
| Exp ID | dispersion | Polymer | load | Solvent | (mg) | (mg) | Medium | 0.5 h | 0.5 h | 1 h | 2 h | 2 h |
| QSA47 | SM | — | — | — | 10.7 | 10.7 | Water | 2.64 | 0.7 | 0.4 | 0.3 | 2.15 |
| QSA48 | | | | | 10.6 | 10.6 | SIF | 6.60 | 0.4 | <0.1 | <0.1 | 6.44 |
| QSA49 | | | | | 11.5 | 11.5 | SGF | 1.24 | 21.9 | 17.2 | 14.6 | 1.23 |
| QSA86 | ASD47 | Carbopol ® | 10 | THF/water (50/50) | 6.1 | 0.6 | Water | 3.55 | 0.5 | 1.8 | 1.5 | 3.58 |
| QSA87 | | | | | 7.1 | 0.7 | SIF | 6.49 | 1.4 | <0.1 | 2.0 | 6.74 |
| QSA88 | | | | | 5.5 | 0.6 | SGF | 1.30 | 64.6 | 92.8 | 108.0 | 1.28 |
| QSA89 | ASD76 | HPMC | 25 | TFE/water (80/20) | 6.6 | 1.7 | Water | 3.11 | 8.5 | 12.1 | 25.4 | 3.09 |
| QSA90 | | | | | 8.1 | 2.0 | SIF | 6.73 | 5.7 | 9.4 | 10.2 | 6.68 |
| QSA91 | | | | | 7.5 | 1.9 | SGF | 1.29 | 179.2 | 167.8 | 136.6 | 1.28 |
| QSA92 | ASD82 | Methyl cellulose | 10 | Chloroform/Ethanol | 6.3 | 3.2 | Water | 3.98 | 2.4 | 1.8 | 2.5 | 4.06 |
| QSA93 | | | | (50/50) | 8.0 | 4.0 | SIF | 6.78 | 1.0 | 1.8 | 1.4 | 6.71 |
| QSA94 | | | | | 12.5 | 6.3 | SGF | 1.29 | 47.9 | 69.7 | 91.8 | 1.30 |
| QSA104 | ASD110 | — | — | THF/water (50/50) | 7.2 | 7.2 | Water | 2.42 | 0.5 | <0.1 | <0.1 | 2.32 |
| QSA105 | | | | | 6.8 | 6.8 | SIF | 6.58 | <0.1 | <0.1 | <0.1 | 6.55 |
| QSA106 | | | | | 8.2 | 8.2 | SGF | 1.22 | 42.0 | 23.0 | 22.8 | 1.27 |

Solubility in Water

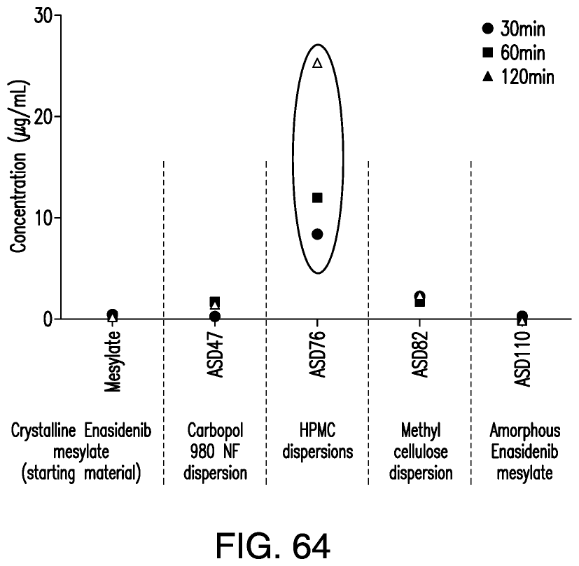

The solubility of Compound 1B at 37° C. in water was about 0.7 μg/mL (FIG. 64). Starting with amorphous Compound 1B similar solubility values were obtained. The best solubility results were found with the amorphous solid dispersion prepared with HPMC (10% drug load). In water, the HPMC dispersion slowly released Compound 1B such that at 120 minutes the highest Compound 1B concentration is achieved. The solubility after 2 hours was about 25 times higher than for Compound 1B. Although a significant improvement in solubility was determined for the HPMC dispersion, only about 5% of Compound 1B present in the dispersion went into solution.

The dispersions prepared with Carbopol® and methyl cellulose did not perform significantly better than Compound 1B. Slightly higher solubility values were found for the methyl cellulose dispersion (about 2 μg/mL).

Solubility in SIF

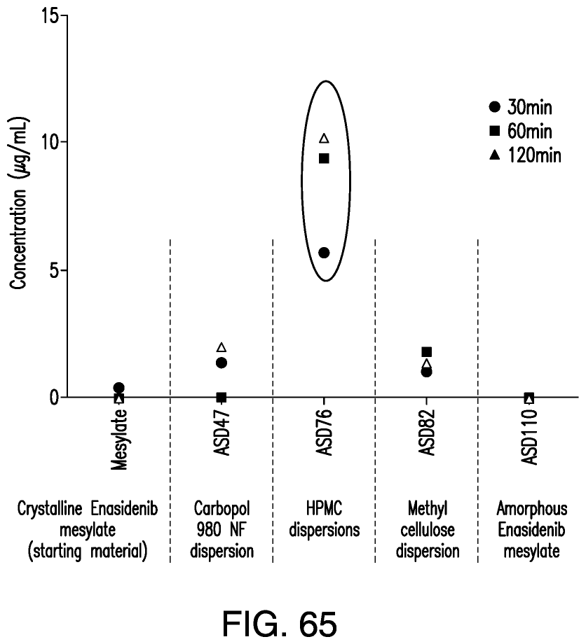

Similar results to those obtained in water were observed in SIF (FIG. 65). Crystalline Compound 1B was poorly soluble at 37° C. in SIF. After ½ hour, the solubility was 0.4 μg/mL and after 1 hour, the concentration had dropped below the detection limit of 0.1m/mL. This observation could be attributed to the disproportionation of the salt and precipitation of the free base.

The release of Compound 1B from the HPMC dispersion followed the same pattern as in water albeit the maximum concentration reached after 2 hours was about 10 μg/mL. This amount corresponded to ±2% of the amount of Compound 1B in the dispersion.

Solubility in SGF

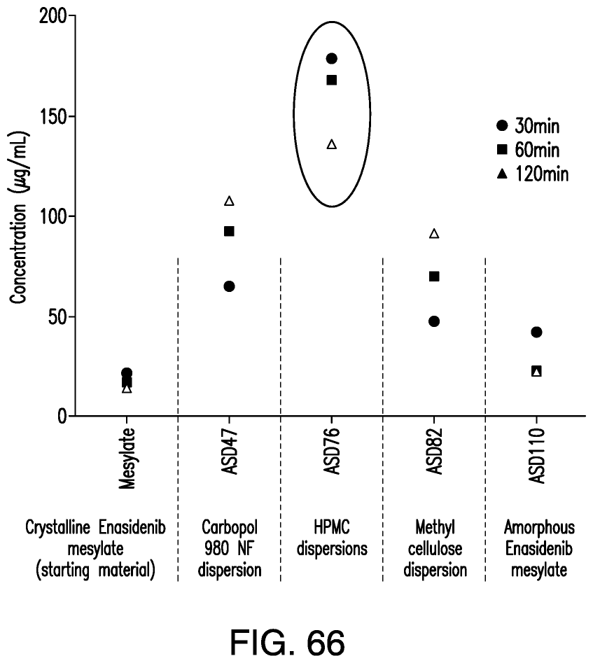

Crystalline Compound 1B reached concentrations close to 22 μg/mL after 30 minutes, which dropped to about 15 μg/mL after 2 hours (FIG. 66).

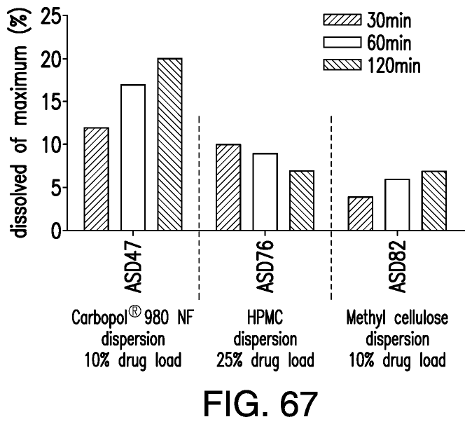

In SGF, a significant increase in solubility was observed for all three dispersion vs the crystalline salt. The best solubility in SGF was reached with the HPMC dispersion. Rapid dissolution resulted in a concentration of about 180m/mL after 30 minutes, which slowly declined to 135m/mL after 2 hours. Dispersions prepared in Carbopol® and methyl cellulose released Compound 1B more slowly and maximum concentrations were reached after 2 hours. Depending on the drug load of the dispersion the maximum amount of drug released is about 20% (FIG. 67). This result was obtained with the 10% Compound 1B load in Carbopol® after 2 hours. From the HPMC dispersion that gave the highest solubility after 30 minutes, 10% of drug was released from the dispersion.

### Example 10: Synthesis of Compound 1A Form A

Slow evaporation of Compound 1A, Form 1 was performed in 10 solvent systems by dissolving 5-10 mg of Compound 1A, Form 1 in 1.0-2.0 mL of acetone or acetonitrile in a 3-mL glass vial. The visually clear solutions were covered with caps and subjected to slow evaporation to induce precipitation at room temperature. The solids were isolated for XRPD analysis after the samples were evaporated to dryness.

The XRPD pattern of Form A is provided in FIG. 68. A differential scanning calorimetry profile (DSC) of Form A is provided in FIG. 69. The DSC profile is characterized by one endotherm at 168.5° C. (onset temperature). A thermal gravimetric analysis (TGA) of Form A is shown in FIG. 69. The weight loss in thermal gravimetric analysis represents a loss of about 3.8% of the weight of the sample as the temperature is increased to about 160.0° C. FIG. 70 provides the ¹H NMR spectrum, which indicates that the molar ratio of acetone to Form A is 0.06 due to the existence of residual solvent.

### Example 11: Synthesis of Compound 1A Form G

About 10 mg of Compound 1A, Form 1 was dissolved in dioxane to obtain a clear solution to obtain a clear solution, followed by addition of n-heptane anti-solvent (ratio of dioxane/n-heptane 1:4). After stirring for about 24 hours, the precipitates were isolated for XRPD analysis and the clear solutions were subjected to slow evaporation to dryness. The solids were isolated for XRPD analysis after the samples were evaporated to dryness.

The XRPD pattern of Form G is provided in FIG. 71. A differential scanning calorimetry profile (DSC) of Form G is provided in FIG. 72. The DSC profile is characterized by two endotherms at 114.3° C. and 204.9° C. (onset temperature). A thermal gravimetric analysis (TGA) of Form G is shown in FIG. 72. The weight loss in thermal gravimetric analysis represents a loss of about 12.4% of the weight of the sample as the temperature is increased to about 161.0° C. When heated to 160° C., Form G converted to an amorphous form, as shown in FIG. 71. FIG. 73 provides the ¹H NMR spectrum, which indicates that the molar ratio of dioxane to Form G is 0.5. The NMR data combined with the TGA data indicate that Form G may be dioxane solvate.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A solid dispersion comprising 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol or a pharmaceutically acceptable salt, solvate, hydrate, stereoisomer, prodrug, or clathrate thereof (Compound 1) dispersed in a solid matrix that comprises a polymer.

2. The amorphous solid dispersion of claim 1, wherein the polymer is selected from the group consisting of a cellulose ester, cellulose ether, polyalkylene oxide, polyacrylate, polymethacrylate, N-vinyl lactam polymer, N-vinyl lactam copolymer, polyacrylamide, vinyl acetate polymer, graft copolymer of polyethylene glycol, polyvinyl caprolactam, polyvinyl acetate, oligosaccharide, and polysaccharide.

3. The amorphous solid dispersion of claim 1, wherein the polymer is selected from the group consisting of hydroxyethyl cellulose, hydroxypropyl methyl cellulose, hydroxypropyl cellulose, methacrylate copolymer, polyethylene glycol 6000, polyvinylpyrrolidone K30, poly(1-vinylpyrrolidone-co-vinyl acetate, polyoxyethylene-polyoxypropylene copolyme, D-α-tocopherol polyethylene glycol 1000 succinate, acrylic polymer, polyvinyl caprolactam-polyvinyl acetate-polyethylene glycol graft copolymer, polyvinyl alcohol-acrylic acid-methyl methacrylate copolymer, polyvinyl alcohol/polyethylene glycol graft copolymer, polyvinyl acetate phthalate, and methyl cellulose.

4. The amorphous solid dispersion of claim 1, wherein the polymer is selected from the group consisting of acrylic polymer, hydroxypropyl methyl cellulose and methyl cellulose.

5. The amorphous solid dispersion of claim 1, wherein the polymer is hydroxypropyl methyl cellulose.

6. The amorphous solid dispersion of claim 1, wherein Compound 1 is present in an amount of from about 5% to about 75% by weight of the amorphous solid dispersion.

7. The amorphous solid dispersion of claim 6, wherein Compound 1 is present in an amount of from about 5%, 10%, 15%, 20%, 25%, 30%, 40% or 50% by weight of the amorphous solid dispersion.

8. The amorphous solid dispersion of claim 6, wherein 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol is present in an amount of from about 10%, 20%, 25%, or 50% by weight of the amorphous solid dispersion.

9. The amorphous solid dispersion of claim 6, wherein 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol methanesulfonate is present in an amount of from about 10%, 20%, 25%, or 50% by weight of the amorphous solid dispersion.

10. The amorphous solid dispersion of claim 1, wherein Compound 1 is substantially non-crystalline.

11. A pharmaceutical composition comprising the amorphous solid dispersion of claim 1, and a pharmaceutically acceptable excipient.

12. A method of treating a disease selected from a hematological malignancy and a solid tumor, each characterized by the presence of a mutant allele of IDH2, comprising administering to a subject having the disease, a therapeutically effective amount of the amorphous solid dispersion of claim 1.

13. The method of claim 12, wherein the disease is characterized by the presence of a mutant allele of IDH2 and the absence of a mutant allele of FLT3.

14. The method of claim 12, wherein the disease is characterized by the presence of a mutant allele of IDH2 and the absence of a mutant allele of NRAS.

15. The method of claim 1, wherein the disease is a hematological malignancy.

16. The method of claim 1, wherein the hematological malignancy is selected from acute myelogenous leukemia, myelodysplastic syndrome, chronic myelomonocytic leukemia myeloid sarcoma, multiple myeloma, lymphoma, angioimmunoblastic T-cell lymphoma, blastic plasmacytoid dendritic cell neoplasm and myeloproliferative neoplasm, each characterized by the presence of a mutant allele of IDH2.

17. The method of claim 1, wherein the hematological malignancy is acute myelogenous leukemia.

18. The method of claim 16, wherein the disease is myelodysplastic syndrome.

19. The method of claim 16, wherein the disease is characterized by the presence of a mutant allele of IDH2 and a mutant allele of at least one second gene, wherein the second gene is selected from the group consisting of ASXL1 and SRSF2.

20. The method of claim 16, wherein the disease is characterized by the presence of a mutant allele of IDH2 and the absence of a mutant allele of at least one other gene, wherein the other gene is selected from the group consisting of KRAS, TP53, SETBP1, U2AF1, TCF3, STAG2, NRAS, JAK2 and BRAF.

21. The method of claim 1, wherein the solid tumor is selected from glioma, melanoma, chondrosarcoma, and cholangiocarcinoma, each characterized by the presence of a mutant allele of IDH2.

22. The method of claim 1, wherein the disease is relapsed or refractory.

23. The method of claim 1, further comprising administering a second active agent.

24. The method of claim 1, wherein the subject is a pediatric patient.

25-37. (canceled)

38. A process for preparing an amorphous solid dispersion of claim 1, comprising (a) providing a solution of Compound 1 and the polymer in a solvent system; and (b) removing the solvent to provide the amorphous solid dispersion.

39. The process of claim 38, wherein the polymer is hydroxypropyl methyl cellulose.

40. The process of claim 38, wherein the solvent system comprises methanol, water, dichloromethane, tetrahydrofuran, acetone, trifluoroethanol, ethanol, or a mixture thereof.

41. The process of claim 38, wherein the solvent system comprises dichloromethane, a mixture of tetrahydrofuran and water, a mixture of methanol and water, a mixture of acetone and methanol, a mixture of 2,2,2-trifluoroethanol and water, or a mixture of chloroform and ethanol.

42. The process of claim 38, wherein the solvent is removed by freeze evaporation.

43. The process of claim 38, wherein the solvent is removed by spray drying.
